(19) United States
(12) Patent Application Publication
(10) Pub. No.: US 2004/0162679 A1
(43) Pub. Date: Aug. 19, 2004

(54) Method for predicting gene potential and cell commitment

(76) Inventor: Linheng Li, Leawood, KS (US)

Correspondence Address: POLSINELLI SHALTON WELTE SUELTHAUS P.C., 700 W. 47TH STREET, SUITE 1000, KANSAS CITY, MO, 64112-1802, US

(21) Appl. No.: 10/430,201

(22) Filed: May 5, 2003

Related U.S. Application Data
(60) Provisional application No. 60/377,383, filed on May 3, 2002.

(51) Int. Cl. G01N 33/48

(52) U.S. Cl. 702/20

(57) ABSTRACT

The present invention relates to methods of associating nucleic acid sequences of unknown function with particular sub-populations, and methods of associating cells of unknown function with particular sub-populations. The method includes collecting hybridization data, developing a gene expression map, and comparing a nucleic acid sequence of unknown function to the gene expression map to determine in what sub-population the gene is expressed. The present invention also relates to kits, arrays, maps, isolated sequences, and clustered sequences.

This application is a non-provisional patent application based on U.S. Provisional Patent Application Serial No. 60/377,383, filed May 3, 2002.

### FIELD OF THE INVENTION

The present invention relates to methods for predicting gene potential and cell commitment, in particular, the present method relates to using known clusters of genes to predict functional and commitment potential of genes and cells of unknown functions. Further, the present invention relates to a family of nucleic acid sequences and genes. The invention relates to arrays, gene clusters, gene maps, and methods for making such products.

### BACKGROUND OF THE INVENTION

Hematopoietic (blood) stem cells (HSCs) are clonogenic cells, which possess the properties of both self-renewal and multilineage potential giving rise to all types of mature blood cells. HSCs are the critical subset of cells in the hematopoietic system that undergo proliferation and differentiation to produce mature blood cells of various lineages while still maintaining their capacity for self-renewal. Hematopoiesis is a dynamic process with significant complexity in which the HSCs give rise to cells of both the myeloid and lymphoid lineages. In addition, HSCs have the ability to self-renew to produce more HSCs. This property allows HSCs to repopulate the bone marrow of lethally irradiated congenic hosts (a host that differs from another with respect to a small chromosomal segment). It is known that HSCs give rise to lymphoid and myeloid cells. Lymphoid cells will further differentiate into T, B, or NK cells. Myeloid cells will further differentiate into granulocyte, monocyte, mega-karyocyte, or erythrocyte cells. Recent reports indicate that murine HSCs also have the potential to trans-differentiate into multiple non-hematopoietic tissues. This suggests that HSCs have greater developmental potential than assumed previously. However, the underlying mechanisms of maintenance of multipotentiality in HSCs remain largely unknown. It is desired to have methods available for understanding such mechanisms.

Differentiation is the complex of changes involved in the progressive diversification of the structure and functioning of the cells of an organism. For a given line of cells, differentiation results in a continual restriction of the types of transcription that each cell can undertake.

Early hematopoiesis is a process of progressive restriction of developmental potential, accompanied with a hierarchical array of self-renewing and multipotent HSCs, non-self-renewing but multipotent progenitors (MPPs), and lineage restricted common lymphoid progenitors (CLPs) or common myeloid progenitors (CMPs). However, the mechanism behind this progressive restriction in developmental potential is not clear. As stated, the hematopoietic system includes HSC, MPP, CLP, and CMP populations. When grouped together, these four cell populations can be referred to as bone marrow stem cells, since all of these populations can be found in the bone marrow.

Early HSC development displays a hierarchical arrangement. The arrangement starts from long-term (LT-) HSCs, which have extensive self-renewal capability. Next is the expansion state, corresponding to short-term (ST-) HSCs (having limited self-renewal ability) and proliferative multipotent progenitor (MPP) (having multipotent potential but no self-renewal capability). MPP is also a stage of priming or preparation for differentiation. MPP differentiates and commits to either common lymphoid progenitor (CLP), which gives rise to all the lymphoid lineages, or common myeloid progenitor (CMP), which produces all the myeloid lineages. During this process, the more primitive population gives rise to a less primitive population of cells, which is unable to give rise to a more primitive population of cells. The intrinsic genetic programs that control these processes, including multipotential, self-renewal, and expansion (or transient amplification) of HSCs, and lineage commitment from MPP to CLP or CMP, remain largely unknown.

The limited number of the hematopoietic stem cells in bone marrow together with an inability to maintain these cells in culture in an undifferentiated state has greatly hindered the characterization of these cells. As such, it is desired to have a method for characterizing the various cell populations, which form the bone marrow stem cell population. In particular, it is desired to have a method that allows for analysis of the intrinsic genetic programs of HSCs.

There are many hypotheses to explain the special behavior of HSCs with regard to the decision-making process by which HSCs choose cell fate between self-renewal and differentiation, arresting and proliferation, or CLP and CMP. These include, for example, the “instructive” model, the “deterministic” model, and the “stochastic” model. The “instructive” model emphasizes the important roles of extrinsic signals such as cytokines in directing cell fate determination. In contrast, the “deterministic” model indicates that it is the intrinsic genetic programs that determine the stem cell fate. The “stochastic” model proposes that cell fate determination is a random event and that the extrinsic signals play a role in selecting one of the possibilities by increasing the cell survival ability or proliferation capacity that favors a particular hematopoietic choice or lineage.

Recently, a hypothesis of “grand-state configuration” of stem cells has been raised. The “grand-state configuration” hypothesis proposes that stem cells maintain a multi-program accessible state determined by their multi-open chromatin structure, allowing accession of different transcriptional factors that can lead to different cell fates. With the progression of development, multi-program accessibility becomes restricted. This model is supported by low levels of expression of multi-lineage affiliated or promiscuous genes in stem cells/progenitor detected by RT-PCR. To further clarify these models and provide insight into the molecular mechanisms that control stem cell fate, it is desired to analyze genome-wide gene expression profiling during the early progression of HSCs self-renewal, expansion, and lineage commitment.

As such, it is believed that the early process of HSC development involves interactions between the intrinsic genetic program and extrinsic signals from surrounding stromal cells. This dynamic progression is accompanied by global changes in gene expression profiles at different stages. These differentially expressed gene sets at different stages of early proliferation and differentiation in turn determine the fate and behavior of HSCs. Methods which elucidate the process of cell commitment can be used to further explain this process. It is also desired to have methods and information which detail and explain the global changes in the gene expression profiles.

A large number of genes that are predominantly expressed in either fetal liver or adult bone marrow HSCs have been discovered and analyzed by gene-expression profiling using array and sequencing technologies. Genes identified in these previous studies are helpful in providing HSC selectively-expressed candidates that might be responsible for self-renewal of HSCs. These genes were identified by subtracting mRNA expressed in HSCs from mRNA expressed in mature cells (such as total bone marrow cells or AA4.1⁻ subset of fetal liver hematopoietic cells). This method provides a limited view of what happens. A majority of multilineage-affiliated genes have been excluded using this process, which may lead to a loss of important information regarding the entire gene expression spectrum in HSCs. This is problematic because it is known that systematic genome-wide profiling of gene expression without pre-excluding multilineage-affiliated genes in functionally homogenous hematopoietic stem and progenitor cells is important for understanding the underlying molecular mechanisms in physiological hematopoietic development.

Finally, it would be useful to have a comprehensive list of all or the majority of genes, ESTs, or nucleic acid sequences expressed in the various bone marrow stem cell populations, including HSC, MPP, CLP, and CMP. Such a list would be useful as a starting point for analyzing self-renewal and commitment, and the mechanisms associated therewith. A list of genes only expressed in HSC would be especially useful.

For the above reasons, it is desired to have a method and system for predicting lineage commitment and self-renewal. It is further desired to have a method for predicting the potential of a gene. It is further preferred to have a method for predicting the fate of a cell or gene. It is also desired to have a method for predicting the function and association of unknown genes or ESTs. In general, it is desired to understand the molecular mechanisms and genetic pathways that regulate adult stem cell development.

### SUMMARY OF THE INVENTION

The present invention relates to a method for analyzing changes in gene expression profiles of cells, wherein the cells are formed from different sub-populations of cells having different differentiation characteristics. In particular, the present invention relates to a method, whereby the activity of genes in each cell population is analyzed to determine which genes are activated and deactivated at each particular cell stage. Through this process, identification of different sets of genes that are predominantly expressed, are identified. This information is useful for predicting the potential of a gene having an unknown function. This information can also be used as part of a method designed to understand the cell fate. As such, the various gene families provide a base line for initiating studies related to understanding the molecular mechanisms and genetic pathways that are regulated in adult cell development. In particular, HSC, MPP, CLP, and CMP cells are well suited for use with the present invention. Genes include all nucleic acid sequences.

The method includes isolating a population of cells and separating the population of cells into discrete cell sub-populations. Thus, the method is initiated by dividing a cell population into sub-cell populations. For example, the stem cells that form the bone marrow hematopoietic system can be divided into HSCs, MPPs, CLPs, and CMPs. However, any cell type can be analyzed if that cell type can be divided into sub-populations having genes that are differentially expressed. The current method only requires two populations, HSCs and MPPs, for example, to determine gene association and potential, as well as cell lineage commitment.

The cell populations are separated using, preferably, cell surface marker techniques. As would be expected, stem cells of the hematopoietic system are well-suited for use in determining gene associations. The stem cells can be stained with any of a variety of immunoflourescent compositions. The compositions or reagents selected are dependent upon whether differentiated cells, which form distinct sub-populations, can be separated. As such, sub-populations may be divided based on distinct surface phenotype, immunological responses, cell cycle status, and proliferation. Other adult stem cell populations can be analyzed with the present method.

Preferably, the method is initiated by staining a population of bone marrow cells to isolate the HSCs, MPPs, CLPs, and CMPs from the bone marrow cell population, as well as the proteins and other constituents associated therewith. The preferred method uses Thy-1^{lo}c-kit⁺Sca-1^{hi}Lin^{-/lo} (KTLS) markers using fluorescence activated cell sorting (FACS). The cells can be from any species, including mammalian and insect species. KTLS markers are useful because sub-populations of bone marrow cells, such as HSCs, can be readily separated. This population of cells is further divided into 2 sub-populations: LT-HSC and MPP cells, according to their abilities to support hematopoiesis and self-renewal. These two populations of cells can be arranged in a lineage according to a progressive loss of the ability to self-renew. The LT-HSC population, representing approximately 0.005% to 0.01% of the bone marrow cells, has extensive self-renewal ability and supports long-term reconstituting ability (>6 months). MPP cells cannot self-renew but can reconstitute bone marrow for less than 4 weeks.

Further division of the sub-population may be necessary to separate populations within a sub-population. A suitable method includes the use of Rhodamine-123. Efflux of dyes, such as Rhodamine-123 (Rh), are used to separate early hematopoietic cells into HSCs and early progenitor sub-populations by flow cytometry. Rh is a mitochondria-binding fluorescent dye, and can be effluxed from the cell by the ABC transporter, P glycoprotein. LT-HSCs, which are relatively quiescent, have high ABC transporter activity, thus the population of cells that stain most weakly with this dye is highly enriched for LT-HSCs. Alternatively, MPP cells can be identified according to the expression levels of lineage-associated antigens such as Mac-1 and CD4.

The isolated population of cells has expressed nucleic acid sequences isolated from the discrete cell sub-populations. The isolated nucleic acid sequences are formed into labeled nucleic acid probes from the expressed nucleic acid sequences. The labeled nucleic acid probes are hybridized with a nucleic acid sequence library on an array, wherein identity and intensity of expression of the expressed nucleic acid sequences are identified to provide gene expression hybridization data. Any method for determining hybridization data can likely be used.

As such, after sub-populations are separated, a clonogenic library is formed. The library will contain activated genes and ESTs (nucleic acid sequences) expressed in each particular population. Probes can be formed from the library. The probes are then analyzed, where ESTs binding to certain probes indicate which genes or ESTs are activated. The hierarchical changes in gene expression profiles of highly purified, functionally homogenous populations of cells, including HSCs, MPPs, CLPs, and CMPs, are examined using an oligonucleotide array. The data provides a global assessment of early hematopoietic development and reveals a hierarchical and asymmetrical distribution of promiscuous gene expression during this process. Gene and EST expression can be visually mapped for each particular population. The gene expression hybridization data can be converted into normalized expression data, whereby change in gene expression between the discrete cell sub-populations is profiled.

The normalized data is statistically analyzed using a variety of organizational and clustering techniques. This includes using Pearson's correlation coefficient and K-means clustering. The gene expression hybridization data is converted into a graphical representation, whereby change in gene expression between the discrete cell sub-populations is profiled.

Expression of genes and ESTs in the HSCs is not just restricted to hematopoiesis-affiliated genes, but also includes genes encoding proteins with functions specified in non-hematopoietic systems, such as neuron, liver, heart, muscle, or endothelial cells. Among the hematopoiesis-affiliated genes, it was found that HSCs primarily expressed myeloid genes but expressed a limited number of lymphoid genes. MPPs express myeloid and increased number of lymphoid genes at low to medium levels, and CMPs and CLPs almost exclusively express the expected profiles of myeloid and lymphoid affiliated genes, respectively. This data clearly supports the principle that promiscuous expression of multiple non-hematopoietic, as well as hematopoietic-affiliated genes, is hierarchically regulated during the process of early hematopoietic development, and correlates with the gene's progressively restricted developmental potential.

As such, a gene or EST (specifically, nucleic acid sequences) of unknown function can be analyzed. In particular, the potential of the gene or EST can be predicted. The gene will be isolated, and expression in at least two sub-populations will be determined. Based on when the gene is expressed, it can be clustered with known genes and compared to the known mapped genes. This will predict the potential function of a gene or EST.

An unknown gene's expression intensity in each of the discrete sub-populations can be identified and compared to the unknown gene's expression pattern with known gene expression patterns in the graphical representation to associate the unknown gene with a group of known genes. This method for characterizing an unknown multilineage-affiliated gene can be summarized as profiling multilineage-affiliated gene expression in discrete cell sub-populations to provide expression data for selected genes in at least two discrete cell populations and comparing an unknown gene's expression data with the expression data.

The same method for determining an unknown gene can be used to determine cell stage commitment. This is done by comparing nucleic acid expression patterns.

A method for developing a gene expression map is practiced. Like above, this is done by isolating at least two sub-populations of cells and obtaining gene hybridization data, including gene identity data and gene expression intensity data, wherein the genes are multilineage-affiliated genes. The method includes normalizing the gene hybridization expression data to provide expression data and filtering the normalized expression data to group genes having similar expression levels. The gene hybridization expression data is converted to a graphical illustration.

An array comprising a plurality of nucleic acid sequences affixed to a substrate can be made. The nucleic acid sequences include representative clusters 1-8 and cumulative clusters 1-100. The array can also be a plurality of nucleic acid sequences affixed to a substrate, wherein the nucleic acid sequences include SEQ ID NOs. 1-4863. Individual groups of sequences SEQ ID NOs. 3428-4863, SEQ ID NOs. 1-821, SEQ ID NOs. 2076-3427, and SEQ ID NOs. 822-2075 can also be affixed to an array.

A kit for characterizing a gene of unknown function, by associating the unknown gene with genes of HSC, MPP, CMP, and CLP reference cell sub-populations, can be made. The kit includes a container and at least one nucleic acid sequence array. An activated label is also included.

The present invention further relates to families of genes and ESTs, which are expressed in the various cell populations. This family is useful for predicting cell fate, and as a tool for studying gene potential and cell commitment.

The present invention relates to a group of nucleic acid sequences for use in determining cell commitment including SEQ ID NOs. 1-4863, and separated groups, SEQ ID NOs. 3428-4863, SEQ ID NOs. 1-821, SEQ ID NOs. 2076-3427, and SEQ ID NOs. 822-2075. Gene clusters have been developed for use in analyzing cell differentiation. Clusters 1-100 are known as cumulative clusters. SEQ ID NOs. 1-4863 form the cumulative gene clusters. Another gene cluster includes representative gene clusters 1-8.

The invention relates to a population of non-hematopoiesis-affiliated genes, which include the genes listed in FIG. 3. Genes listed as upregulated in CMP and the genes listed in FIG. 4D are part of the present invention. Genes upregulated in HSC, and genes listed in FIG. 4A are part of the invention. Genes upregulated in CLP, and listed in FIG. 4C, are part of the invention. Genes upregulated in MPP, and listed in FIG. 4B, are part of the invention. A gene cluster map for use in analysis of multilineage-affiliated genes can be made. The map includes an axis related to at least two cell populations, an axis comprising normalized gene expression values, and a plot of genes clustered according to K-means clustering.

A computer system is part of the present invention and includes a processor, storage media for storing a database, and a program module, executable by the processor. The program module includes computer readable program code for effecting the steps illustrated in FIGS. 9-11.

A method for developing a gene expression map is practiced. It includes: isolating at least two sub-populations of cells, obtaining gene hybridization data, including gene identity data and gene expression intensity data, wherein the genes are multilineage-affiliated genes; and, converting the gene hybridization expression data to normalized gene expression data.

The present invention is advantageous because a family of genes and ESTs are known, which are associated with HSCs and can be used as a tool for predicting cell function. Other gene families are also developed which are associated with MPP, CLP, and CMP. The present invention is further advantageous because a method is provided for predicting gene potential and cell commitment. The present method can be used to form gene expression maps. Further, the present invention can be used to predict stem cell fate or commitment. Hypothetically, this method could be used as part of a method to control stem cell determination and development. Related to this is a family of genes expressed in HSC. As such, the present invention provides insight into the stem cell commitment and development process.

### BRIEF DESCRIPTION OF THE DRAWINGS

The application file contains at least one photograph executed in color. Copies of this patent application publication with color photographs will be provided by the Office upon request and payment of the necessary fee.

FIG. 1 shows isolation and characterization of hematopoietic stem and progenitor cells;

FIG. 1A is a schematic illustration of hematopoietic development, with the surface markers used for purifying each population of cells indicated, also, the correlation coefficient between each pair of these populations is illustrated;

FIG. 1B shows the cell cycle status of purified HSCs and MPPs using Rho123 test;

FIG. 2 shows a global view of the gene expression patterns of 4,863 genes clustered by the K-means clustering method, using Eisen's Cluster and a TreeView software (Eisen Lab, Berkeley, Calif.), with the expression levels of genes presented according to a colored gradient scale from the highest (red) to the lowest (green) representative genes for each sub-population are included

FIG. 2A is representative of HSC,

FIG. 2B is representative of MPP,

FIG. 2C is representative of CLP, and

FIG. 2D is representative of CMP;

FIG. 3 shows promiscuous gene expression of non-hematopoiesis-affiliated genes in an HSC, with a list of non-hematopoiesis-affiliated genes predominantly expressed in HSCs;

FIG. 4 shows a global view of the gene expression patterns of 4,863 genes clustered by the K-means clustering method and visualized using Eisen's Treeview software;

FIG. 4A is representative of HSC,

FIG. 4B is representative of MPP,

FIG. 4C is representative of CLP, and

FIG. 4D is representative of CMP;

FIG. 5 shows clusters of genes categorized by the expression patterns in purified stem and progenitor cells, a Spotfire® software program (Somerville, Mass.) was used to visualize the changes in expression levels of each gene during hematopoietic development, the vertical axis represents the normalized gene expression values;

FIG. 5A represents genes that are predominantly expressed in HSCs and down-regulated in MPPs, CLPs, and CMPs;

FIG. 5B represents genes that are up-regulated in MPPs;

FIG. 5C represents genes that are highly expressed in CLPs;

FIG. 5D represents genes that are highly expressed in CMPs;

FIG. 6 illustrates verification of Affymetrix® (Santa Clara, Calif.) data shown in i) using single cell RT-PCR: ii) relative expression levels of MHCII.2A, ESK, and CyclinA2 in each progenitor subset by array analyses; and, iii) results of single cell RT-PCRs for each target gene in HSCs and MPPs;

FIG. 7A shows the expression profiles of genes with known functions during hematopoietic development with changes in lineage and non-lineage-related gene expression shown;

FIG. 7B shows changes in chromatin-structure-related gene expression; and,

FIG. 7C is a schematic illustration of the correlation between promiscuous gene expression and development potential;

FIG. 8 is a gene expression map showing the up-regulation and down-regulation of gene clusters among cell sub-populations for the gene expression data in Table 4;

FIG. 9 is a flow diagram detailing a method for characterizing an unknown gene;

FIG. 10 is a flow diagram detailing a method showing how genes are clustered;

FIG. 11 is a flow diagram detailing a method showing how expression maps are formed; and,

FIG. 12 is a scattering diagram.

### SEQUENCE LISTING

The Sequence Listing, in computer readable form (CRF), is submitted on compact disc, and is hereby incorporated by reference into this patent application. A total of three compact discs are being submitted.

Compact Disc No. 1—Sequence Listing—CRF, contains a file named IP-010.5T25.txt, with 4,416 KB, which was created on Apr. 24, 2003.

Compact Discs No. 2 and 3—Sequence Listing and Table 1, containing files named IP-010.5T25.txt, with 4,416 KB, created on Apr. 24, 2003, and Table 1.txt, created Apr. 29, 2003, with 14,761 KB, and labeled Copy 1 and Copy 2.

### DESCRIPTION OF THE INVENTION

The present invention relates to a method for analyzing, determining and predicting cell differentiation and commitment, as well as gene expression patterns in a cell population having distinct sub-populations. In particular, the present invention relates to a method for developing gene expression maps, which provide a model of the up-regulation and down-regulation of genes in various cell sub-populations. The present invention relates to a method for predicting gene potential. Related to the present method are families of genes and ESTs, which are expressed in HSC, MPP, CLP, and CMP. The models demonstrate hierarchical changes in expression. More particularly, the present invention relates to a method for analyzing hematopoietic stem cell differentiation and lineage commitment. A gene expression map can be developed for each distinct cell sub-population, specifically HSCs, MPPs, CLPs, and CMPs. The map can also be developed to show gene regulation in the various sub-populations. As such, the gene expression maps can be used to predict gene function, or the particular sub-population of an unknown cell. The maps can also be used as part of a method to predict lineage commitment. Thus, the present invention relates to a method for predicting an unknown gene's function by analyzing the expression patterns of the gene, in view of the previously discussed and developed expression maps.

Any of a variety of cells may be analyzed using the present method, as long as the particular cell population can be divided into distinct cell sub-populations, wherein genes are either up-regulated or down-regulated in the distinct sub-populations as the cells develop from one population to the next. Bone marrow cells in early hematopoiesis are well-suited for analysis with the present method because they include four distinct sub-populations, LT-HSC, MPP, CLP, and CMP. The LT-HSC is the most primitive of the sub-populations and is characterized by its self-renewal capabilities. The LT-HSC either renews or evolves first into a short term (ST)-HSC, followed by differentiation into a MPP cell. MPPs have reduced self-renewal capabilities. The MPP cells then differentiate into either CMP or CLP cells. The CLPs and CMPs are the most advanced progenitors of the cell populations. As cells progress from HSC to CLP or CMP, the ability to self-renew is lost and lineage commitment occurs. The cells can be HSC, transition, and differentiated cells which include adult, embryonic, neonatal, fetal, liver, bone marrow, splenic, and lymphoid stem cells. Transition cells are those that are between HSC and differentiated cells.

Each of the sub-populations will have distinct gene expression patterns where the genes are either up or down-regulated. Thus, the genes, ESTs, or more particularly nucleic acid sequences, activated in each population are distinct and, as a group, can be distinguished from groups of genes in other sub-populations. This is important because the gene expression patterns provide clues as to how cell fate is determined. In particular, this helps to provide insight as to whether an HSC ultimately commits to HMLA, including CMP or CLP, or NHA differentiation. The gene expression patterns can also be used to predict gene potential. Collectively, this information can be used as the basis for a method for promoting a particular cell commitment. Additionally, other stem cell populations can be analyzed with the present method. Adult stem cells are well suited for analysis with the present method.

The HSCs or related cells to be analyzed can be derived from any of a variety of species, including any Animalia member, in particular, insects, mammals, or humans. Also, the cells can be derived from a variety of tissues. Preferably, the cell population used is derived from bone marrow or fetal liver tissue. The initial cell population will preferably be somatic bone marrow stem cells.

To form a gene expression map or categorize the genes, and to associate groups of genes with a particular sub-population, it is first necessary to separate the HSC population or selected cell population into the distinct sub-populations. Any of a variety of methods may be used to separate the population of cells into the distinct cell sub-populations. It is, however, important that the sub-populations be divided and separated. An available method for separating or dividing the sub-populations of a population of cells is initiated by obtaining a sufficient supply of, for example, bone marrow or fetal liver tissue cells, which will contain the various sub-populations. An amount of bone marrow or fetal liver tissue cells equal to at least 2.0×10⁴ cells should be obtained. Higher amounts may be used. The amount should be sufficient so that there is a sufficient amount of each sub-population to form a clonogenic library.

To divide the cells into sub-populations, the cells can be marked with various fluorescent materials or agents and are then separated using a flow cytometry method, which is a technology that utilizes an instrument in which particles in suspension are stained with a fluorescent dye and passed in single file through a narrow laser beam. The fluorescent signals emitted when the laser excites the dye are electronically amplified and transmitted to a computer. The computer is programmed to instruct the flow cytometer to sort the particles having specified properties into collecting vessels. In this way, the cells are divided. Flow cytometry is desirable because it is a high throughput technique that will allow for large numbers of cells to be analyzed and separated.

Flow cytometry involves the use of a fluorescence-activated cell sorter (FACS) device, which will sort the separate cell populations. These devices can be purchased from a variety of manufacturers, including Becton Dickinson Immunocytometry Systems of San Jose, Calif. As such, the devices are configured with various lasers to identify the formats or dyes.

Various fluorescent compositions can be attached to antibodies and other cell markers. It is preferred to use multiple formats or fluorescent compositions so that when cells are analyzed with the FACS sorter, various colors will indicate specificity to antibodies, or cell markers. In this way, cells can be distinguished and sorted. The available formats include, for example, fluorescein isothiocyanate (FITC), R-phycoerythrin (PE), and allophycocyonin (APC). Obviously, other compositions can be used which can be detected by a FACS device and attached to a cell marker.

It is most preferred to use a multicolor flow cytometric analysis to separate the cells. Multicolor flow cytometric analysis enables the simultaneous detection of the light-scattering characteristics (forward and side-scattered light signals) of cells, as well as their expressed levels of two or more intracellular and/or cell surface antigens that are defined by immunofluorescent staining. In this way, multicolor flow cytometric analysis enables characterization of individual cells having a variety of distinct cellular characteristics and functions. These characteristics may define a cell's activation status, lineage, subset identity, the capacity to bind cells and tissues, or migrate to sites of inflammation.

In the present method, signaling molecules that selectively adhere to the receptors on the surface of the cell are used to identify differentiated sub-populations. The signaling molecule is attached to another molecule (or the tag) that has the ability to fluoresce or emit light energy when activated by an energy source, such as an ultraviolet light or laser beam.

As such, a suspension of tagged cells (cells bound to the cell surface markers which have fluorescent tags) is sent under pressure through a very narrow nozzle—so narrow that cells must pass through one at a time. This is part of the FACS system. Upon exiting the nozzle, cells then pass, one-by-one, through the light source (laser), and then through an electric field. The fluorescent cells become negatively charged, while non-fluorescent cells become positively charged. The charge difference allows the cells to be separated from other cells. This results in a population of cells that have all of the same marker characteristics.

Surface markers are preferred for use in initially separating the cells into sub-populations. In the preferred method, the bone marrow stem cells, or selected cells, are separated by first incubating the cells with monoclonal antibodies against lineage positive markers. This will create two populations of cells: one that is Lin⁺ and the other that is Lin⁻. Lin⁺ cells are cells in which lineage commitment has resulted. Lin⁻ cells have not committed. A suitable method involves separating, the Lin⁺ cells from the Lin⁻ cells by incubation with antibody coated Dynabeads®, whereby the Lin⁺ cells will attach to the Dynabeads®, and the Lin⁻ cells will pass through. Other methods, however, can be used to separate the Lin⁻ cells.

The Lin⁻ cells are then further separated. The preferred method for isolating the Lin⁻ cells is the use of a KTLS method and kit. The cells are stained with APC conjugated c-Kit PE, conjugated Sca-1 fluorescein isothiocyanate, and Biotin/Sa-PerCPCy5.5 conjugated Thy-1. The population can then be sorted using FACS so that a population of c-kit⁺ Thy-1^{lo} Lin⁻ Sca-1⁺ cells are isolated. This is known as a KTLS population. The “+” and “−” indicate whether a cell is positive for or negative for the particular stain. As such, dependent on the desired separation, these characteristics can be selected, based on the particular cell population.

Sca-1 is a biotinylated monoclonal antibody specific for Sca-1. Lin relates to lineage markers, such as the CD family of cell surface antigens. Thy-1 is present in T-cells and is a marker. Kit also relates to cell surface antigens, such as CD117. As such, any of a variety of kits or testing protocols for staining the cells can be purchased from various providers, such as Pharmingen (San Diego, Calif.).

Stem cell markers are given short-hand names based on the molecules that bind to the stem cell surface receptors. For example, a cell that has the receptor stem cell antigen-1 on its surface, is identified as Sca-1. A stem cell antigen is a cell-surface protein on bone marrow (BM) cells, indicative of HSC. A c-Kit is a cell-surface receptor on bone marrow cell types that identifies HSC. With regard to lineage surface antigens, there are 13 to 14 different cell-surface proteins that are markers of mature blood cell lineages (Lin⁺). Detection of Lin⁻ cells assist in the purification of HSC and hematopoietic progenitor populations. Thy-1 is a cell-surface protein. Negative or low detection of Thy-1 is suggestive of HSC. As would be expected, the selected markers are dependent upon the specific cell population to be isolated.

Once the KTLS Lin⁻ cell population is isolated, it is necessary to further divide this sub-population into two distinct sub-populations. This can be accomplished using any of a variety of methods including a Rhodamine-123 method to stain the cells. Rhodamine is a mitochondrial binding fluorescent dye that is effluxed from the cell by the ABC transporter, P glycoprotein. Rhodamine-123 (R-302; FluoroPure Grade (Molecular Probes, Inc, Eugene, Oreg.), R-22420) is widely used as a structural marker for mitochondria and as an indicator of mitochondrial activity. Additionally, it is a cell-permeant, cationic, fluorescent dye that is readily sequestered by active mitochondria without inducing cytotoxic effects. Uptake and equilibration of Rhodamine-123 is rapid (a few minutes) compared to dyes such as DASPMI, which may take 30 minutes or longer. Viewed through a fluorescein long-pass optical filter, the mitochondria of cells stained by Rhodamine-123 appear yellow-green. Viewed through a tetramethylrhodamine long-pass optical filter, however, these same mitochondria appear red. A FACS sorter is again used to separate the Rh^{lo} from the Rh^{hi} cells. Rh^{lo} cells will be LT-HSCs. The Rh^{hi} cells will be MPP cells. Thus, two distinct sub-populations are separated.

The Lin⁺ cells will be separated from Dynabeads® to which antibodies were attached. The lineage positive cells, are the cells which are enriched for the Lin commitment cells. These include CLP and CMP cells. As such, these two sub-populations of cells can be isolated, initially, by separating the lineage negative from the lineage positive cells. The cells can, again, be isolated according to cell surface markers, wherein the CLPs, are of a c-kit^{lo}, sca-1^{lo}, IL-7R⁺, FcR ID and the CMP is IL-7R⁻, Sca-1⁻, c-kit⁺, CD34⁺, FcR^{lo}. The IL-7R, CD34, and FcR, are all antibodies. While the above methods are preferred, any of a variety of methods can be used to separate cell sub-populations.

After separation of the populations, gene expression and identity are determined. From each sub-population, the RNA is extracted, specifically the mRNA. As would be expected, the presence of mRNA indicates genes in the particular cell sub-population, which are being expressed. Any of a variety of methods can be used to extract the mRNA, as long as it is readily obtained, and can be used to form a clonogenic library, such as a cDNA or cRNA library. Note, that it is generally necessary to have a minimum of 50,000 cells per sub-population to obtain a linear application of mRNA, using a T7 promoter-based RNA amplification method. It is preferred to extract approximately 300 nanograms (ng) of mRNA from the isolated cell populations.

A cDNA library can be formed from the isolated mRNA of each sub-population. RNA molecules are exceptionally labile and difficult to amplify in their natural form. For this reason, the information encoded by the RNA is converted into a stable DNA duplex (cDNA) and then is inserted into a self-replicating lambda vector. Once the information is available in the form of a cDNA library, individual processed segments of the original genetic information can be isolated and examined. The cDNA library can be formed by using any of a variety of known methods and kits, including the ZAP expression cDNA synthesis kit, manufactured by Stratagene (LaJolla, Calif.). The cDNA will be synthesized by reverse transcription using a superscript and then by DNA synthesis using Klenow DNA polymerase, for example. These products can be purchased from Invitrogen® (Carlsbad, Calif.) or Stratagene®, for example. As would be expected, any of a variety of methods can be used to form the clonogenic library.

The cDNA library can be amplified by cloning it into any of a variety of expression vectors, with the amount of cDNA isolated from the vectors sufficient to produce a cDNA library. After extraction of the mRNA, it is necessary to form a clonogenic library. The cRNA library is synthesized in vitro from a linearized cDNA template using T7 RNA polymerase in the presence of the cap analogue 7 mGpppG. Resultingly, four clonogenic libraries, which are related to gene expression are isolated and developed.

The clonogenic libraries, preferably four, are then analyzed with a bioinformatics program, which will detect expression levels of the genes in each sub-population. In particular, specific genes expressed in each population will be isolated. Specifically, each cDNA or cRNA library will be hybridized with known arrays. A preferred array is an MG-U74 oligonucleotide array. This is manufactured by Affymetrix® Gene Chip Company.

Base-pairing (i.e., A-T and G-C for DNA; A-U and G-C for RNA), or hybridization, is the underlining principle of the DNA or oligonucleotide microarray. An array is an orderly arrangement of samples. It provides a medium for matching known and unknown DNA or RNA samples based on base-pairing rules and automating the process of identifying the unknowns. An array experiment can make use of common assay systems, such as microplates or standard blotting membranes, with samples deposited on them either manually or by utilizing robotics. In general, arrays are described as macroarrays or microarrays, the difference between them being the size of the sample spots. Macroarrays contain sample spot sizes of about 300 microns or larger and can be easily imaged by existing gel and blot scanners. The sample spot sizes in microarray are typically less than 200 microns in diameter, and these arrays usually contain thousands of spots. Microarrays require specialized robotics and imaging equipment.

DNA microarray, or DNA chips, are fabricated by high-speed robotics, generally on glass but, sometimes, on nylon substrates, for which probes with known identity are used to determine complementary binding, thus allowing massively parallel gene expression and gene discovery studies. An experiment with a single DNA chip can provide information on thousands of genes simultaneously. Generally, a “probe” is the tethered nucleic acid with known sequence, whereas a “target” is the free nucleic acid sample whose identity/abundance is being detected.

There are two major application forms for the DNA microarray technology: 1) identification of sequence (gene/gene mutation); and 2) determination of expression level (abundance) of genes. In the present method, it is preferred to identify expressed sequences and the expression level.

There are two variants of the DNA microarray technology, in terms of the property of arrayed DNA sequence with known identity. In Format 1, a cDNA (500˜5,000 bases long) is immobilized to a solid surface, such as glass, using robot spotting and exposed to a set of targets, either separately or in a mixture. The second option involves an array of oligonucleotide (20˜80-mer oligos) or peptide nucleic acid (PNA) probes which are synthesized either in situ (on-chip) or by conventional synthesis, followed by on-chip immobilization. The array is exposed to labeled sample DNA, hybridized, and the identity/abundance of complementary sequences are determined. Many companies manufacture oligonucleotide-based chips using alternative in-situ synthesis or depositioning technologies.

Analysis software, provided by Affymetrix, for example, can convert the raw hybridization intensities into expression level measurements (“average difference” in Affymetrix terms) for each gene or nucleic acid sequences. The expression levels are based on a comparison between the hybridization signals of a perfect match (PM) and a mismatch (MM). Negative values are obtained, if the MM value was higher than the PM value, making it difficult to compare the expression patterns between two or more conditions when one of the conditions was a negative value. Therefore, all negative values can be converted to a positive 20, using 20 as the background level. This data conversion method is used to permit estimation of the number of genes expressed in each sub-population of cells. A gene is defined to be “expressed” when the expression level of that gene is determined to be greater than 100. Expression level is measured by the affinity of binding of cRNA sequences derived from expressed genes to a group of select representative oligonucleotides on the gene chip. Alternatively, gene expression level is measured by binding of multiple copies of labeled cRNA binding to the array.

The genes in the microarray data are considered as differentially expressed and can be subsequently screened for clustering analysis. Preferably, the genes are filtered based upon a certain expression level. Genes which are not sufficiently expressed, are eliminated from the analysis. Preferably, the genes are analyzed with a gene filter of the following parameters: |yⱼ₍ₘ₎−yⱼ₍ₗ₎|>100 and yⱼ₍ₘ₎/yⱼ₍ₗ₎>2 for j=1, . . . ,n, where yⱼ₍ₘ₎ and yⱼ₍ₗ₎ are the order statistics with yⱼ₍ₗ₎≦ . . . ≦yⱼ₍ₘ₎ for the j^{th} gene. This filtering criterion considers either simultaneously or sequentially the absolute difference (>100) of the gene expression levels and the fold change (>2-fold) of the expression levels for each gene (>100). Thus, 4,863 genes were selected for clustering analysis, including 137 initial seeds.

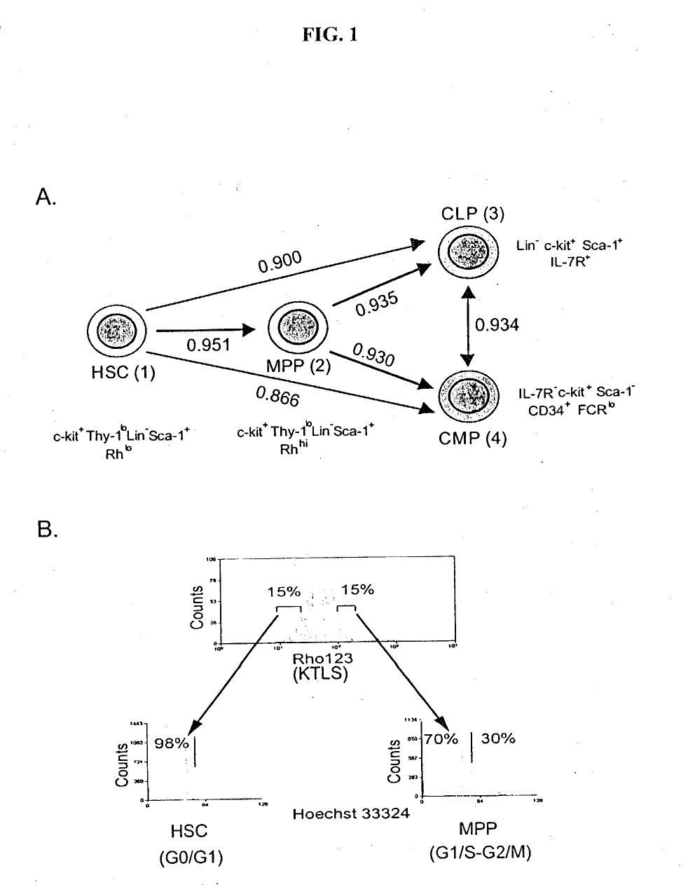

There are multiple ways to interpret the data. Any of a variety of clustering and hierarchial measurements can be used. To understand the pair-wise relationship between each population of cells in terms of the gene expression intensity and diversity, Pearson's Correlation Coefficient can be calculated. The Pearson's formula is applied to the raw expression level data. The lineage relationship is reflected by the correlation coefficient (r), between HSCs (defined as subscript 1) and MPPs (defined as subscript 2), CLPs and CMPs (defined as subscripts 3 and 4), is r₁₂=0.951 (FIG. 1a), indicating a significant positive linear correlation between the gene expression intensity and a measure of gene diversity between HSCs and MPPs. Similar calculations yielded r₁₃=0.900, r₁₄=0.866, r₂₃=0.935, r₂₄=0.930, and r₃₄=0.934, indicating linear correlation of gene expression intensity and gene diversity measurements between HSCs vs. CLPs, HSCs vs. CMPs, MPPs vs. CLPs, MPPs vs. CMPs, and CLPs vs. CMPs, respectively. The numerical Pearson's correlation values reflect the physiological hierarchical relationship among these purified populations, as shown in FIG. 1a.

Pearson's Correlation Coefficient is explained as follows: yⱼₖ represents the expression level of the j^{th} gene in k^{th} sample, here k=1, . . . m, and j=1, . . . ,n, with m=4, and n=24,000 in sample data. Let k=1 correspond to the sample gene expression observed in LT-HSC, k=2 in MPP, k=3 in CLP, and k=4 in CMP. The Pearson's Correlation Coefficient between any two samples is given by the following equations:

for i≠k, and 1≦i,k≦m,

where

$${\overline{y}}_{k} = {\sum }_{j = 1}^{n} {y}_{jk} / n , and {s}_{k} = \sqrt{{\sum }_{j = 1}^{n} {( {y}_{jk} - {\overline{y}}_{k} )}^{2} / ( n - 1 )}$$

are the mean and standard deviation of the k^{th} sample, respectively. As an expression level below 20 may not be confidently measured (3 Tamayo), a threshold value of 20 is assigned to an expression level that is below 20.

The closer the resultant coefficient is to a value of one, the more significant the correlation. The calculation showed that the distance between HSC and MPP is r₁₂=0.950 (see FIG. 1A), indicating a very high positive linear correlation between the expression levels in LT-HSC and MPP. This numerical score indicated that highly expressed genes in LT-HSC tended to have large overall intensities in MPP. Moreover, this result indicated that the LT-HSC and MPP sub-populations are similar in their gene intensity patterns. Similar calculations yielded the following data: r₁₃=0.900, r₁₄=0.866, r₂₃=0.937, r₂₄=0.933, and r₃₄=0.936 (see FIG. 1A), which indicated linear correlations between gene expressions in LT-HSC and CLP, LT-HSC and CMP, MPP and CLP, MPP and CMP, and CLP and CMP, respectively.

These numerical measures of correlation matched with known biological situations of the hierarchical sequential of HSC proliferation and differentiation. MPP falls into a pivotal population downstream of LT-HSCs and upstream of either CLP or CMP within a relatively close distance to HSCs (0.950) and almost equal distances to either CLP or CMP (0.937, 0.933). The developmental distance between MPP and CLP or CMP is similar to that between CLP and CMP (0.936).

In summary, the Pearson's calculations established a lineage relationship between the various cell sub-populations. The lineage relationship was reflected by the correlation coefficients. The Pearson's computation quantified a physiological hierarchical relationship among the cell sub-populations. Thus, these results illustrate differential gene expression across the four hematopoietic sub-populations characterized. As such, genes associated with these sub-populations can be grouped and analyzed.

Once the hybridization data is collected, the expression patterns need to be analyzed. Genes with similar expression behavior (up-regulation or down-regulation under a similar condition) are likely to be related functionally, so that the relative expression patterns among genes in a targeted population of cells are compared. To analyze the patterns of gene expression, a variety of clustering methods can be used, including self-organization maps, hierarchical clustering, and K-means cluster. K-means is the most preferred.

The K-means clustering method gathers genes into groups according to similarity of expression patterns among target populations. In particular, it allows for the selection of initial seeds according to known features (genes with known biological functions); then the genes are grouped around selected seeds by K-means clustering. According to known important roles played in hematopoiesis, 137 genes were selected as the initial seeds. Genes that passed the initial screening filter (1, with absolute expression level >100 in at least one condition; 2, with >2 fold changes between at least two conditions) were used for further analysis.

The K-means clustering method groups items together according to the similarity of the items. The similarity/dissimilarity of the ith and jth genes are given by the Euclidean distance between the two observations:

$${r}_{ik} = \frac{{\sum }_{j = 1}^{n} ( {y}_{ji} - {\overline{y}}_{i} ) ( {y}_{jk} - {\overline{y}}_{k} )}{( n - 1 ) {s}_{i} {s}_{k}} ,$$

for i≠k, and 1≦i,k≦m,

where

$${\overline{y}}_{k} = {\sum }_{j = 1}^{n} {y}_{jk} / n , and {s}_{k} = \sqrt{{\sum }_{j = 1}^{n} {( {y}_{jk} - {\overline{y}}_{k} )}^{2} / ( n - 1 )}$$

are the mean and standard deviation of the k^{th} sample, respectively.

This method is designed to group observations into a collection of K clusters. The value of K can be determined either in advance or as a part of the clustering procedure. As such, the clustered genes can form a map for use in analyzing expression.

After gathering the hybridization signal intensity data, it can then be analyzed using imaging processing software by Eison. Alternatively, software developed by Spotfire® may be used. This provides an illustration of when a gene is up-regulated and down-regulated from one sub-population to another. As such, a clear method, or illustration, is provided, which shows and correlates gene expression to lineage development. In particular, a map is developed for each sub-population, with the map illustrating all genes expressed in the particular sub-population. This data can be combined to illustrate gene up-regulation and down-regulation from one population to the next. As such, a couple of different types of maps are provided for comparison purposes. One map is a grouping of all genes that are up-regulated in a particular sub-population. The other is an illustration of how the genes are turned on and off.

The potential for unknown genes, can be predicted possibly by comparing the gene to the maps mentioned herein. Conversely, this can be done by comparing data. The gene can be analyzed in all four sub-populations to determine when it is and is not expressed. Expression levels of the gene will then be compared to expression levels of known genes. Depending upon when the gene is up-regulated and down-regulated, this will allow for the prediction of the gene's potential function by comparing it with known genes and their functions. As such, the expression patterns of the gene will associate it with other known genes.

Further, a cell, in particular, a bone marrow cell, can be isolated, and the lineage commitment of such cell can be determined. This is done by comparing the genes, which are up-regulated in such cell, with expression patterns of known cells.

Isolated groups of genes are also related to the present invention. Related to the present invention are families of genes and ESTs. The isolated groups will be those genes that are up-regulated or down-regulated during or in a particular cell sub-population. As such, there are gene maps for HSC (SEQ ID NOs 3428-4863), MPP (SEQ ID NOs 2076-3427), CLP (SEQ ID NOs 1-821), and CMP (SEQ ID NOs 822-2075).

Table 1, which discloses the above genes, is submitted on compact disc (3 copies), is incorporated by reference into this patent application.

Thus, hematopoietic stem cells (HSCs) have self-renewal capacity and multilineage developmental potentials. HSC development progresses from quiescent long-term HSCs to proliferative multipotent progenitor, and to differentiating common lymphoid/myeloid progenitors. The molecular mechanisms that determine the pluripotent potential, and early lineage commitment of HSCs, remain largely unknown. Using Affymetrix® MG-U74 A and B chips representing 24,000 genes and ESTs, changes in the gene expression profiles are illustrated from developmental progression to adult murine HSCs (SEQ ID NOs. 3428-4863). It was observed that a promiscuous expression of non-hematopoietic-affiliated and hematopoietic multilineage-affiliated genes in HSCs occurred. During the progression of HSC proliferation and differentiation, the gene expression profile becomes less promiscuous and this correlated with a progressively reduced developmental potential. This observation implied that hematopoietic stem cell pluripotent potential is determined by its multi-program accessibility. As such, a method is provided for genome-wide expression profiling.

This investigation will tell us what it is about these genes and proteins that determines the type of cells they become such as T and B lymphocytes, erythrocytes, monocytes, megakaryocytes, and granulocytes, what causes cells to undergo self-renewal, expansion, or maturation, and how cells migrate to different parts of the body.

A gene matrix can be constructed by adhering and affixing the isolated genes and expressed sequence tag (EST) cluster nucleic acid sequences from the cell sub-populations in a particular tissue lineage pathway onto a solid phase matrix or support. Suitable multilineage cell tissue can include, but are not limited to hematopoietic, nerve, muscle, kidney, and liver. The Affymetrix.RTM.417.TM. Arrayer and 427.TM. Arrayer can be used to deposit densely packed nucleic acid arrays on glass slide matrixes. Suitable solid phase matrices that can be used are silica or silica-based materials, inorganic glass, functionalized glass, polymers, plastics, resins, polysaccharides, carbon, metals, polymerized Langmuir Blodgett film, Si, Ge, GaAs, GaP, SiO₂, SiN₄, polytetrafluoroethylene, polyvinylidenedifluoride, polystyrene, polycarbonate, or combinations thereof.

The nucleic acid sequences are placed in contact with the slide, washed, and dried for use in assays. In this manner, separate gene arrays for each cell sub-population in a multilineage differentiation pathway can be created for subsequent characterization of sub-populations. Alternatively, the gene arrays for all sub-populations of cells associated with a multilineage pathway can be placed on the same slide matrix. Affymetrix GeneChip MU-U74 (version 2-arrays A and B (Affymetrix, Santa Clara, Calif.) also can be used.

A nucleic acid library on a solid phase matrix or support can be used. Specifically, a separate glass solid phase matrix can be constructed containing a library of nucleic acid sequences associated with HSC, MPP, CMP, or CLP genes.

Solid phase matrices can also be constructed that include nucleic acid sequences associated with a combination of hematopoietic cell sub-populations. A fifth glass matrix can be prepared that contains HSC and MPP sequences selected from a group consisting of SEQ. ID. NO. 2076-4863. A sixth glass matrix can be prepared that contains MPP and CMP sequences selected from a group consisting of SEQ. ID. NOs. 822-3427. An seventh glass slide can be made that contains MPP and CLP sequences selected from a group consisting of SEQ. ID. NOs. 1-821 and 2076-3427. A eighth glass slide can be prepared that contains HSC, MPP, CMP, and CLP sequences selected from a group consisting of SEQ. ID. NO. 1-4863.

A hematopoictic cell differentiation test kit can be constructed that includes the following components: a container, a hematopoietic cell microarray or an Affymetrix GeneChip MU-U74 Array A and B; a 96-well microtiter plate array of 0.2 ml microamp tubes; fluorescence-labeled (e.g., fluorescein, rhodamine) monoclonal antibodies to HSC, MPP, CMP, and CLP markers (e.g., Thy-1, Sca-1, Lin); four sets of Dynabead packed affinity columns for purification of HSC, MPP, CMP, and CLP cells, respectively; biotinylated RNA probe positive control standards which specifically bind to HSC, MPP, CMP, and CLP regions on the arrays; preserved HSC, MPP, CMP, and CLP control cell lysates; gene specific primers for at least four genes corresponding to each four sub-populations of cells for RT-RNA replication, R-phycoerythrin-conjugated streptavidin; antisense biotinylated control cRNA (bioB, bioC, bioD, and cre); a HPRT RNA transcript positive control for RT-PCR reactions, biotin-N-hydroxysuccinimide ester; biotinylated-cRNA controls for HSC, MPP, CMP, and CLP sub-populations, Qiagen RNAeasy columns, lysis buffer containing 0.5% Triton X-100, ethidium bromide stain for electrophoresis, reference gene expression maps for HSC, MPP, CMP, CLP sub-populations and transition cells; and computer software for data analysis.

The kit's included computer software can perform the following functions: conversion of raw hybridization intensity data into gene expression levels based on computation between hybridization signals of perfect match and mismatch pairs; conversion of negative values to positive values; computation of “expressed” gene numbers, based on establishment of a minimum expression level; Pearson correlation coefficient computation for gene distance similarities and differences; prescreening and selection of genes using a screening filter for subsequent clustering analysis; normalization of gene expression level standard deviation results; K-means gene clustering for grouping of cumulative clusters 1-100 of Table 3 and representative clusters 1-8 of Table 4; conversion of normalized gene expression standard deviation results to graphical representation, and color fingerprinting among cell sub-population categories utilizing Spotfire visualization of gene expression map fingerprint patterns.

The kit user will provide a FACS sorter or micro-manipulator for separation and purification of hematopoietic cells and deposition into tubes of 96 well plate arrays; hematopoictic cells to be characterized; fluorescence detection instrumentation; Dulbecco's MEM or RPMI-1640 culture media; HEPES, cell buffers; micro-pipettors and tips; electrophoresis apparatus; and agarose gels.

The foregoing test kit can be used in characterization of both unknown hematopoietic genes and unknown cells along the HSC to MPP to CMP/CLP differentiation pathways. Briefly, bone marrow, spleen, liver, lymph node, or other hematopoietic stem cell sources are separated by FACS sorting or Dynabead affinity column separation into HSC, MPP, CMP, and CLP sub-populations of interest. Upon separation, isolated cells from a specific sub-population (e.g., HSC-MPP transition cells) are placed individually in a 96 well microtiter plate array using either FACS instrument sorting or micromanipulation. The HSC-MPP transition cells are lysed in cell lysis buffer, and subjected to first and second round PCR amplification to obtain amplified cRNA copies of HSC and MPP genes of interest utilizing gene-specific forward and reverse primers. Lysates containing PCR amplified c-RNA copies of HSC, MPP, CMP, and CLP genes respectively can be included in parallel with the HSC-MPP transition cell lysates as positive controls.

The amplified cRNA from replicate wells derived from the HSC-MPP transition cell and the four sub-population control cells can be biotinylated with the biotin-NHS ester and mixed with antisense biotinylated control cRNA. Then the biotinylated-specific cRNA and/or the antisense control cRNA for each respective sub-population is hybridized with the nucleic acid sequences on the microarray or the Affymetrix GeneChip. Strepavidin-conjugated phycoerythrin is added to enable detection of the gene expression level for each corresponding gene on the HSC and MPP gene expression map. The reference gene expression map is created by use of the kit's software, as provided in Example 27. Characterization of the expression pattern of a plurality of genes (e.g., at least 5 genes) from the HSC-MPP transition cell will yield a gene expression fingerprint map that may include characteristics of the HSC positive control cell and the MPP positive control reference cell genes. The gene expression fingerprint map characterizing the HSC-MPP transition cell can be compared with the reference gene expression fingerprint maps obtained from both (1) the reference HSC and MPP sub-population cell lysates included in the aforementioned biotinylation procedure, and (2) the kit's biotinylated cRNA control reagents provided for corresponding reference HSC and MPP cell sub-populations. In addition, the HSC-MPP transition cell gene expression map may be compared against the kit's included printed version of the reference HSC, MPP, CMP, CLP, and transition cell gene expression maps.

In this manner, both the isolated individual HSC-MPP transition genes and transition cells that express these genes can be characterized and identified. Similarly, isolated individual MPP to CMP transition genes and cells, as well as MPP to CLP transition genes and cells, can be characterized and identified. Moreover, individual cells within the HSC, MPP, CMP, and CLP sub-population categories may be further characterized.

For characterization of a particular unknown HSC-MPP transition gene, amplified cRNA from the isolated HSC-MPP cell can be obtained and biotinylated. This is mixed with antisense biotinylated control cRNA and hybridized with nucleic acid sequences on the microarray. Strepavidin-conjugated phycoerythrin is added, and a light source excites fluorescence photon emission from the phycoerythrin label. The instrument's detector collects the emitted light photon signal and the instrument's software converts the raw photon data into gene expression intensity signal data. The instrument's software can further convert the unknown gene expression intensity signal data to normalized gene expression data expressed as a numerical standard deviation (s.d.) value. The software then compares the unknown gene expression numerical s.d. value for the same gene's expression numerical s.d. value in the control reference HSC, MPP, CMP, CLP, and transition cell sub-populations. Based upon the unknown gene's expression numerical s.d. value software then determines similarities or differences in comparison with the control reference cell values to characterize and identify the unknown gene. Moreover, when this gene characterization procedure is executed for a plurality of genes obtained from several HSC-MPP transition cells, the kit's software can be utilized to obtain groupings of gene clusters that characterize and identify a particular HSC-MPP transition cell sub-population.

For characterization of an isolated unknown transition cell, the above procedure can be followed for each individual gene. In this manner, an unknown transition cell's gene expression map may be constructed for a plurality of individual genes. As described previously, this unknown cell's gene expression map can be compared with the reference gene expression maps for known reference HSC, MPP, CMP, CLP, and transition cell sub-populations. Based upon these map comparisons, the unknown transition cell can then be characterized and identified.

### EXAMPLES

### Example 1

To determine expression patterns of genes and how a cell commits to a specific lineage, it was necessary to separate differentiated sub-populations of cells. In particular, a method was practiced, which separated sub-populations of bone marrow stem cells, whereby LT-HSCs, MPP, CLP, and CMP populations were separated.

4.7×10⁹ bone marrow cells were collected from the femurs and tibias of 60 C57B6-J mice (6-8 weeks old). These bone marrow cells were incubated with rat monoclonal antibodies against lineage-positive cell surface markers (Pharmingen) including CD34, IL-7R, Fcγ RII/III, and CD2. Lineage negative (Lin^{-/lo}) cells were enriched by twice depleting lineage-positive cells through incubation with antibody-coated (sheep antirat IgG) Dynabeads® M-450 (Dynal Biotech, Oslo, Norway). This created two populations of cells, lineage positive (Lin⁺) and lineage negative (Lin⁻).

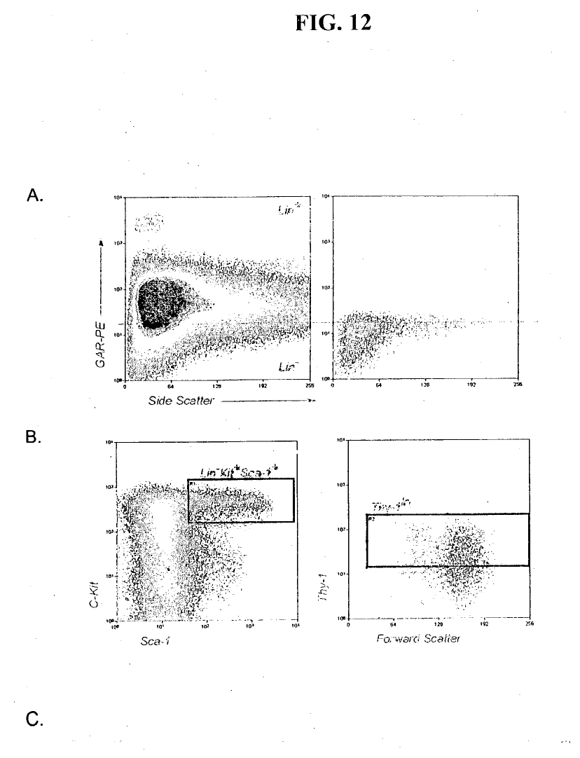

The Lin^{-/lo} cells were stained with Sca-1 fluorescein isothiocyanate (FITC) APC-c-Kit PE conjugated Sca-1, and Biotin/Sa-PerCPCy5.5 conjugated Thy-1. The KTLS cell population was sorted as c-kit⁺, Sca-1⁺, and Thy-1^{lo} using FACS. The printout of the data is shown in FIG. 1a. Thy-1⁻ was defined by isoform IgG2bκ and the Thy-1^{hi} population could be seen when the R1 gate, shown in FIG. 12, was moved to the Sca-1⁻ position.

Thus, the mouse hematopoietic stem cells (HSCs) were isolated with c-kit⁺Thy-1^{lo}Lin^{-/lo}Sca-1^{hi} (KTLS) markers using fluorescence activated cell sorting (FACS). The HSCs represent about 0.05% of mouse bone marrow cells and these cells can fully reconstitute all blood cell elements. The population of cells isolated with KTLS was heterogeneous and contained three subpopulations: LT-HSCs, ST-HSCs, and MPP. The LT-HSC sub-population supports long-term reconstituting ability (>6 months); ST-HSCs briefly contribute to hematopoiesis (6-8 weeks); and MPP cells reconstitute bone marrow for less than 4 weeks. Thus, sub-populations of stem cells were isolated using cell surface markers.

### Example 2

The Lin⁺ cells of Example 1 were necessarily separated from Lin⁻ because lineage commitment had already occurred. CLP and CMP are the first differentiation branches of cell lineage commitment from MPP. Approximately 80,000 cells of each of the CLP (c-kit^{lo}, Sca-1^{lo}, IL-7R⁺) and CMP (IL-7R⁻, Sca-1⁻, c-kit⁺, CD34⁺, FcR^{lo}) were isolated following previously reported FACS procedures and using a FACS sorter. Thus, a second group of cells were isolated using cell surface markers.

### Example 3

A second process was performed to separate the sub-populations of HSCs that were previously isolated in Example 1. Rhodamine-123 (Rh) was used to separate the KTLS cells of Example 1 into LT-HSC and early progenitor sub-populations by flow cytometry. Rh is a mitochondria-binding fluorescent dye, and can be effluxed from the cell by the ABC transporter, P glycoprotein. LT-HSCs, which are relatively quiescent, have high ABC transporter activity; thus, the population of cells that stain most weakly (Rh^{lo}) with this dye are highly enriched for LT-HSCs. In contrast, intermediate (Rh^{itm}) and highest (Rh^{hi}) staining of Rh, relate to two populations of cells that are enriched with ST-HSCs and MPP cells. Using Rh staining, the KTLS cells were separated into Rh^{lo} and Rh^{hi}, populations, as illustrated in FIG. 1B. In order to avoid a potential interference from the intermediate staining of Rh (Rh^{itm}) cells which were enriched with ST-HSCs, a symmetrical portion of cells containing either the 15% highest staining or the 15% lowest staining for Rh was chosen. The 80,000 cells representing LT-HSCs and MPP isolated using this protocol represent 0.002% of the total nucleated bone marrow cells obtained from the 60 C57B-6J mice, of Example 1.

Thus, a variety of different known methods were used to isolate various HSC sub-populations. In particular, incubation with monoclonal antibodies directed against lineage positive members was used, followed by a KTLS method. The KTLS cells were further isolated using Rh staining. In this way, populations of LT-HSC, MPP, CLP, and CMP cells were separated.

### Example 4

A competitive repopulation assay to confirm the functional differences between the Rh^{lo} and Rh^{hi} populations of cells was performed. The result demonstrated that the engraftment rate using Rh^{lo} cells was much higher than that using Rh^{hi} cells post-transplantation. In addition, the Rh^{lo} cells could support hematopoiesis for up to 6 months post-transplantation and were able to reconstitute the bone marrow in a secondary transplantation. The Rh^{hi} cells, which gave rise to both myeloid and lymphoid lineages, engrafted the bone marrow for less than 4 weeks. This result demonstrated that the two sorted cell populations were distinct: the Rh^{lo} population of cells was enriched for LT-HSCs and the Rh^{hi} population of cells was enriched for MPP.

Although the competitive re-population assay demonstrated that Rh^{lo} and Rh^{hi} KTLS cells were functionally distinct, the cell cycle states remained uncharacterized. Rh^{lo} KTLS has been known to be enriched with cells that are in a relatively arrested or quiescent state. In contrast, Rh^{hi} KTLS are enriched cells that are actively cycling. To confirm this observation, the Rh^{lo} and Rh^{hi} KTLS cells were stained together with Hoechst 33324 dye. The flow result shows that the Rh^{lo} KTLS population of cells was enriched with cells that were in the G0/G1 phase (98%); in contrast, only 70% Rh^{hi} KTLS population of cells were in G1 phase; and the rest were in the S/G2/M phases (FIG. 1B). These sorted Rh^{lo} and Rh^{hi} KTLS populations most likely reflect the developmental stages of HSCs, with Rh^{lo} KTLS representing the LT-HSCs that were relatively quiescent and were in the self-renewal compartment. The Rh^{hi} KTLS represent MPP cells that were highly proliferative and in the expansion compartment.

### Example 5

Total RNA was extracted from 8×10⁴, from each of the four purified sub-populations of the previous Examples by the Trizol method. A minimum of 50,000 cells is required to obtain a linear amplification of RNA using T-7 promoter based RNA amplification. The amount of RNA was measured using Microplate SpectraMax® (Molecular Devices Corp., Sunnyvale, Calif.). Approximately 300 ng of total RNA was obtained from each cell population.

cDNA and the corresponding cRNA were synthesized following the manufacturer's procedure. A cDNA library, known as “A” was constructed from 1.3×10⁶ cells using the ZAP expression cDNA synthesis kit following the manufacturer's procedure (Stratagene). Briefly, total RNA was isolated from the sub-population of cells using Trizol Reagent (BRL). The cDNA was synthesized first by reverse transcription using Superscript II (Invitrogen) and then by DNA synthesis using Klenow DNA polymerase (Invitrogen). The cDNA inserts were cut with EcoRI and XhoI restriction enzymes and cloned into the EcoRI/XhoI sites of λZAPExp vector. The plasmids (pBK; Strategene, LaJolla, Calif.) bearing the cDNA inserts were excised from their parent vector λZAPExp using helper phage according to the manufacturer's protocol. The primary cDNA library A contained 36,000 clones. cDNA libraries were made therefrom. cRNA was purified using Qiagen RNeasy Columns (Qiagen® Valencia, Calif.), and fragmented to sizes of 35-200 bases. cDNA libraries were made for each sub-population of cells.

### Example 6

Analysis of gene expression was conducted using the clonogenic libraries formed from the isolated cell sub-population discussed in Example 5. The gene expression in the HSCs, MPPs, CLPs, and CMPs was measured by using MG-U74 set oligonucleotide arrays A and B. The Affymetrix® GeneChip MU-U74 (Version 2) arrays A and B (Affymetrix®) cover approximately 6,000 murine known genes, and 18,818 EST Unigene cluster sequences. Equal amounts (5 μg) biotinylated-labeled cRNA derived from each population of cells were mixed with anti-sense biotinylated control cRNA (bioB, bioC, bioD, and cre) and were then individually hybridized with Chips A and B. The chips were then washed, stained, scanned, and normalized (enabling comparisons of data between chips) following the standard procedure (Affymetrix®). As such, gene expression was identified and the gene expression intensity was measured.

A replicate result of the hybridization data for isolated HSC and MPP cell sub-populations from two independent experiments, wherein each well reproduced and demonstrated similar expression patterns. The average of these two results were used for data analysis. Due to the extremely limited numbers of CLPs in murine bone marrow, only one set of hybridization data for CLP and CMP was obtained.

Streptavidin (SA) (a biotin binding protein)-conjugated PE was used to detect the hybridization signal intensity. Replicate hybridization results for HSCs and MPP from two independent experiments were obtained. Light emission signals were collected by a detector, and signal intensities were computed that quantified the binding of PE-SA-label to biotinylated cRNA probes hybridized with nucleic acid sequences on the array. These signal intensities were used to determine gene expression intensity and gene diversity. Average signal intensity values were derived for both HSC and MPP cell sub-populations. Thus, raw data on gene expression identity was obtained. Further, data on the intensity of expression was obtained.

### Example 7

Analysis software, provided by Affymetrix, converted the raw hybridization intensities into expression level measurements (“average difference” in Affymetrix terms) for each gene or nucleic acid sequences. The expression levels were based on a comparison between the hybridization signals of a perfect match (PM) and a mismatch (MM). Negative values were obtained if the MM value was higher than the PM value, making it difficult to compare the expression patterns between two or more conditions when one of the conditions was a negative value. Therefore, all negative values were converted to a positive 20, using 20 as the background level. This data conversion method was used to permit estimation of the number of genes expressed in each sub-population of cells. A gene was defined to be “expressed” when the expression level of that gene was determined to be greater than 100. Expression level was measured by the affinity of binding of cRNA sequences derived from expressed genes to a group of select representative oligonucleotides on the gene chip. Alternatively, gene expression level was measured by binding of multiple copies of labeled cRNA binding to the array.

### Example 8

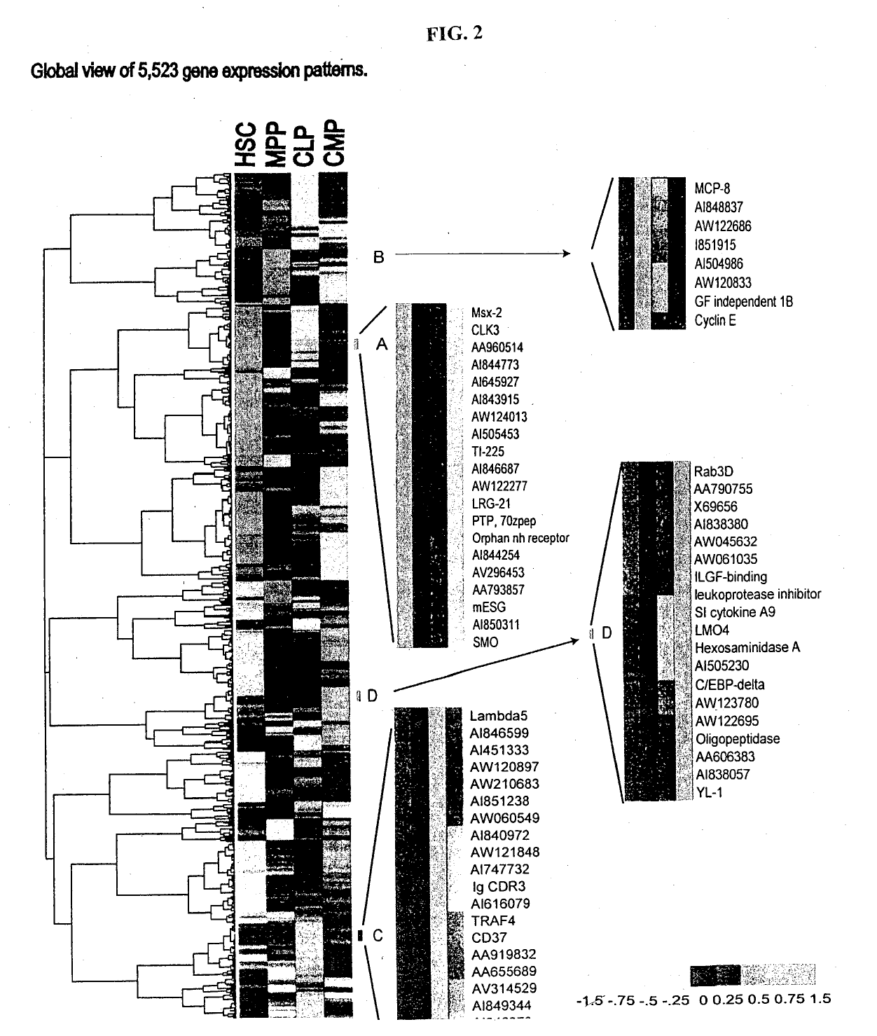

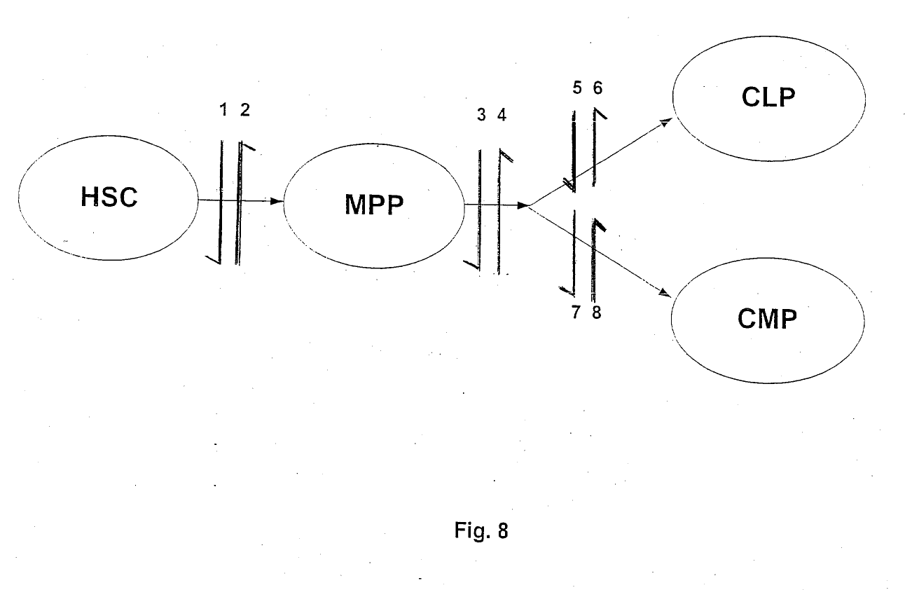

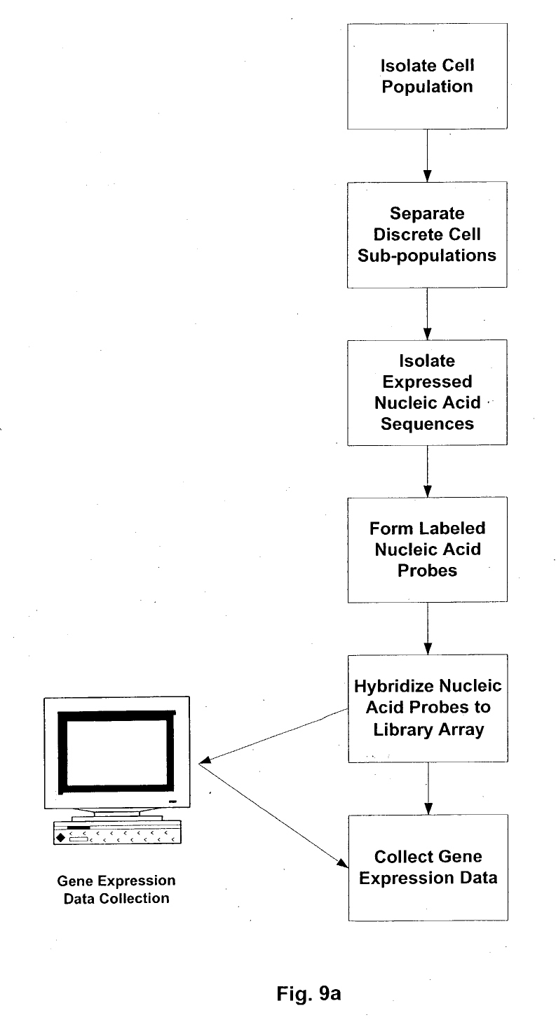

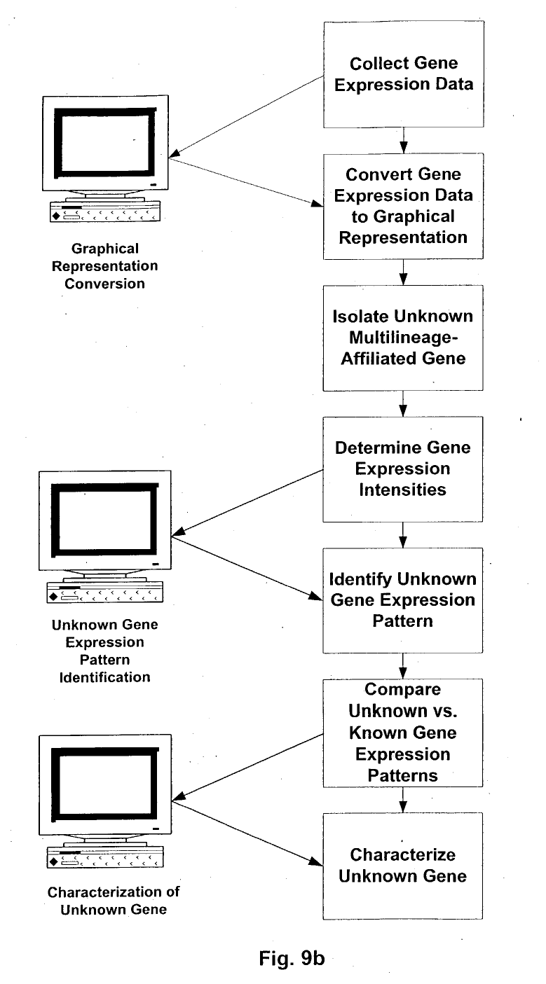

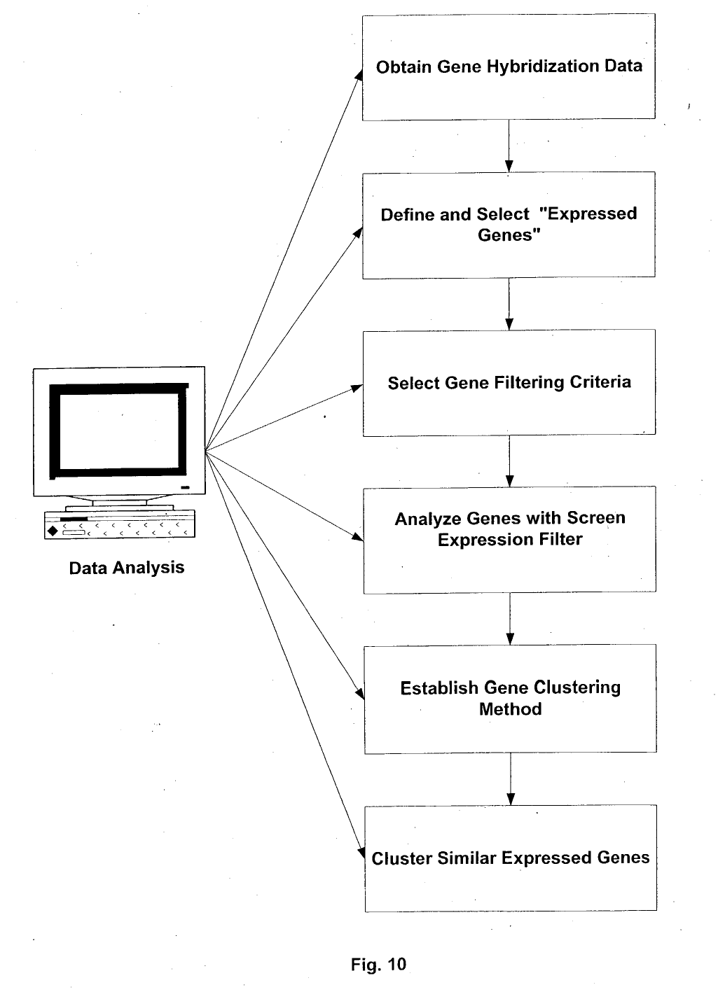

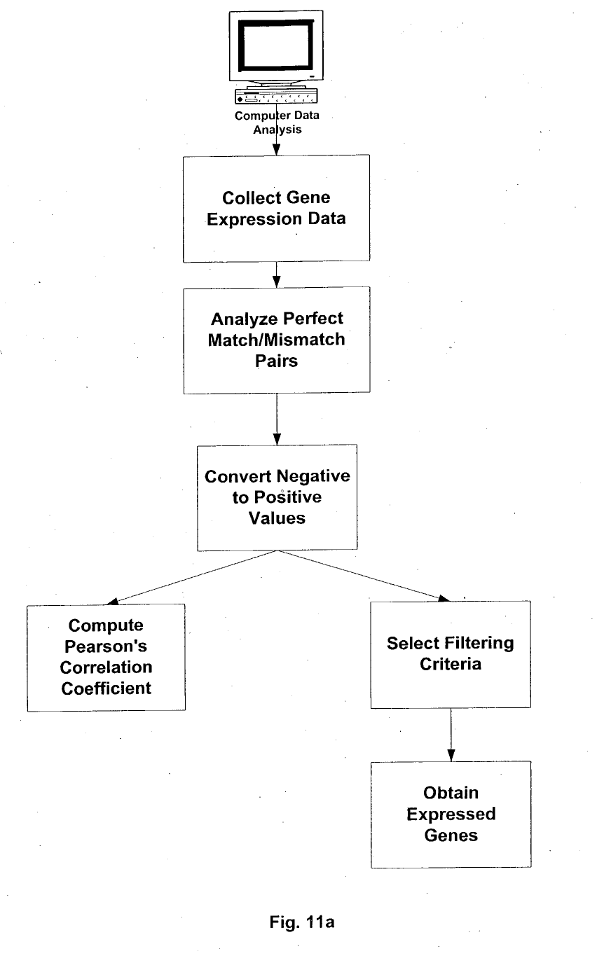

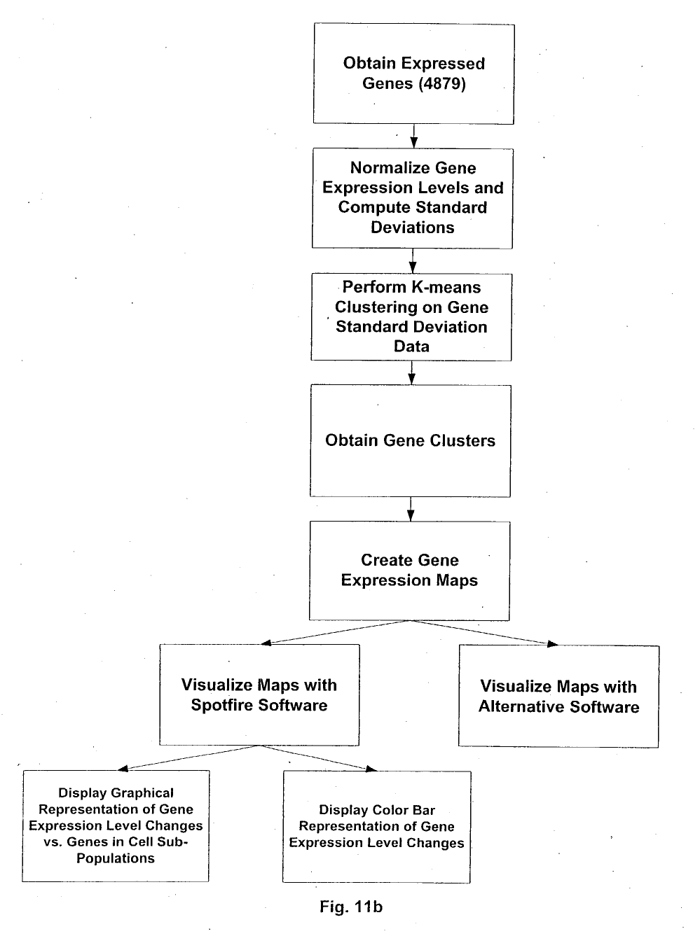

Using a scattering plot (FIG. 12), gene expression intensity and diversity among the 4 sub-populations of cells, LT-HSCs, MPP, CLP, and CMP (FIGS. 2, 3, and 4), were compared. The signal intensity derived from LT-HSCs was used as the reference point. As FIGS. 2, 3, and 4 show, the diversity of gene expression in each population of cells provided a global view of the gene expression changes. These expression changes reflected the stepwise progression from LT-HSC to MPP and then from MPP to either CLP or CMP, and are consistent with the illustrations in FIG. 1A and FIG. 8.

### Example 9

To understand the pair-wise relationship between each population of cells in terms of the gene expression intensity and diversity, Pearson's Correlation Coefficient was calculated. The Pearson's formula was applied to the raw expression level data of Example 7. The lineage relationship, reflected by the correlation coefficient (r), between HSCs (defined as subscript 1) and MPPs (defined as subscript 2), CLPs and CMPs (defined as subscripts 3 and 4), is r₁₂=0.951 (FIG. 1a), indicating a significant positive linear correlation between the gene expression intensity and a measure of gene diversity between HSCs and MPPs. Similar calculations yielded r₁₃=0.900, r₁₄=0.866, r₂₃=0.935, r₂₄=0.930, and r₃₄=0.934, indicating linear correlation of gene expression intensity and gene diversity measurements between HSCs vs. CLPs, HSCs vs. CMPs, MPPs vs. CLPs, MPPs vs. CMPs, and CLPs vs. CMPs, respectively. The numerical Pearson's correlation values reflect the physiological hierarchical relationship among these purified populations, as shown in FIG. 1A.

Pearson's Correlation Coefficient is explained as follows: yⱼₖ represents the expression level of the j^{th} gene in k^{th} sample, here k=1, . . . m, and j=1, . . . ,n, with m=4, and n=24,000 in sample data. Let k=1 correspond to the sample gene expression observed in LT-HSC, k=2 in MPP, k=3 in CLP, and k=4 in CMP. The Pearson's Correlation Coefficient between any two samples is given by the following equations:

$${r}_{ik} = \frac{{\sum }_{j = 1}^{n} ( {y}_{ji} - {\overline{y}}_{i} ) ( {y}_{jk} - {\overline{y}}_{k} )}{( n - 1 ) {s}_{i} {s}_{k}} ,$$

for i≠k, and 1≦i, k≦m,

where

$${\overline{y}}_{k} = {\sum }_{j = 1}^{n} {y}_{jk} / n , and {s}_{k} = \sqrt{{\sum }_{j = 1}^{n} {( {y}_{jk} - {\overline{y}}_{k} )}^{2} / ( n - 1 )}$$

are the mean and standard deviation of the k^{th} sample, respectively. As an expression level below 20 may not be confidently measured (3 Tamayo), a threshold value of 20 is assigned to an expression level that is below 20.

The closer the resultant coefficient is to a value of one, the more significant the correlation. The calculation showed that the distance between HSC and MPP is r₁₂=0.950 (see FIG. 1A), indicating a very high positive linear correlation between the expression levels in LT-HSC and MPP. This numerical score indicated that highly expressed genes in LT-HSC tended to have large overall intensities in MPP. Moreover, this result indicated that the LT-HSC and MPP sub-populations are similar in their gene intensity patterns. Similar calculations yielded the following data: r₁₃=0.900, r₁₄=0.866, r₂₃=0.937, r₂₄=0.933, and r₃₄=0.936 (see FIG. 1A), which indicated linear correlations between gene expressions in LT-HSC and CLP, LT-HSC and CMP, MPP and CLP, MPP and CMP, and CLP and CMP, respectively.

These numerical measures of correlation matched with known biological situations of the hierarchical sequential of HSC proliferation and differentiation. MPP falls into a pivotal population downstream of LT-HSCs and upstream of either CLP or CMP within a relatively close distance to HSCs (0.950) and almost equal distances to either CLP or CMP (0.937, 0.933). The developmental distance between MPP and CLP or CMP is similar to that between CLP and CMP (0.936).

In summary, the Pearson's calculations established a lineage relationship between the various cell sub-populations. The lineage relationship was reflected by the correlation coefficients. The Pearson's computation quantified a physiological hierarchical relationship among the cell sub-populations. Thus, these results illustrate differential gene expression across the four hematopoietic sub-populations characterized. As such, genes associated with these sub-populations can be grouped and analyzed.

### Example 10

Analysis was performed to determine the number of genes that were expressed in each cell population by selecting genes with expression levels above the cut-off line defined by a compensation method. The method is described in Example 10. The results of the present analysis are as follows:

TABLE 2

| | HSCs | MPPs | CLPs | CMPs |
|---|---|---|---|---|
| Presence of expression | | | | |
| Genes with expression levels passed cutoff line (more than 100) | 10677 (43%) | 10488 (42%) | 10466 (42%) | 10455 (42%) |
| ESTs (Affymetrix database) | 8600 | 8402 | 8438 | 8471 |
| Known genes | 2077 | 2086 | 2028 | 1984 |
| Genes affiliated with non-hematopoietic tissues | 58 | 58 | 58 | 58 |
| Genes with normalized value more than 0.3 | 43 (74%) | 22 (37%) | 13 (22%) | 4 (7%) |
| Genes affiliated with hematopoiesis | 312 | 312 | 312 | 312 |
| Genes with normalized value more than 0.3 | 133 (42%) | 95 (30%) | 141 (45%) | 80 (26%) |
| Low-level expression (more than 100 but less than 300) | 5891 (24%) | 5840 (23%) | 5826 (23%) | 5844 (23%) |
| Differential expression | | | | |
| Genes passed screening filter (FIG. 2) | 4863 | 4863 | 4863 | 4863 |
| Genes dominantly expressed in each population | 824 (16%) | 723 (13.8%) | 527 (11%) | 665 (12.7%) |
| ESTs (Affymetrix database) | 666 | 609 | 398 | 545 |
| Known genes | 158 | 114 | 129 | 120 |
| Genes affiliated with non-hematopoietic tissues | 48 | 48 | 48 | 48 |
| Genes with normalized value more than 0.3 | 35 (73%) | 18 (37%) | 10 (20%) | 4 (8.3%) |
| Genes affiliated with hematopoiesis | 162 | 162 | 162 | 162 |
| Genes with normalized value more than 0.3 | 63 (39%) | 42 (26%) | 67 (41%) | 40 (25%) |

The total number of genes on the chips was identical at 24 818 for HSCs, MPPs, CLPs, and CMPs. A gene was defined to be present if the expression level of a given gene was greater than 100. Genes that are highly expressed were defined as if a normalized value more than 0.3, here 0.3, is a normalized value (see the color bar for reference).

As shown in Table 2, about 42% of genes on the chips were detectable in each cell sub-population. Among these, approximately 23% of genes were expressed at low levels in each sub-population of cells. The expression levels of surface markers used for sorting each sub-population (e.g., c-Kit, Sca-1, and IL-7R) determined by the array analysis were consistent with the definition of each sub-population based on FACS analysis, which verifies the quantification of gene expression in the assay (FIG. 1A). In addition, the result of analyzing representative genes using single-cell RT-PCR also verified the microarray analysis (results on file). The pair-wise relationship between HSCs and MPPs, represented by the Pearson correlation coefficient (γHSC-MPP), was 0.951 (FIG. 1A), indicating a significant positive linear correlation of gene expression intensity and diversity between these populations. Likewise, γHSC-CLP and γHSC-CMP were 0.900 and 0.866, and γHSC-CLP and γHSC-CMP were 0.935 and 0.930, respectively. Thus, the numerical correlation values correctly reflect the hierarchical relationship among these purified populations in physiologic hematopoiesis (FIG. 1). Within approximately 2000 known genes that passed the cut-off line, 2 groups of genes were classified as either hematopoiesis- or nonhematopoiesis-affiliated genes according to their tissue-specific expression or functions (Table 2). These genes are shown in FIGS. 2, 3 and 4.

### Example 11

The genes in the microarray data were considered as differentially expressed and were subsequently screened for clustering analysis with a gene filter given by the following equation: |yⱼ₍ₘ₎−y_{j(l)|>}100 and yⱼ₍ₘ₎/yⱼ₍ₗ₎>2 for j=1, . . . ,n, where yⱼ₍ₘ₎ and yⱼ₍ₗ₎ are the order statistics with yⱼ₍ₗ₎≦ . . . ≦yⱼ₍ₘ₎ for the j^{th} gene. This filtering criterion considers either simultaneously or sequentially the absolute difference (>100) of the gene expression levels and the fold change (>2-fold) of the expression levels for each gene (>100). Thus, 4,863 genes were selected for clustering analysis, including 137 initial seeds.

### Example 12

Based on an assumption that genes with similar expression behavior (up-regulation or down-regulation under a similar condition) are likely to be related functionally, the relative expression patterns among genes in the targeted population of cells were compared. To analyze the patterns of gene expression, a variety of clustering methods were used, including self-organization maps, hierarchical clustering, and K-means clustering.

After the comparison of the clustered results, it was observed that the genes grouped by K-means cluster were more related to each other as judged by their known biological functions. That is because K-means clustering gathered genes into groups according to similarity of expression patterns among target populations. Particularly, this method allowed the selection of initial seeds according to known features (genes with known biological functions); then the genes were grouped around selected seeds by K-means clustering. According to known important roles played in hematopoiesis, 137 genes were selected, as stated in the Description of Invention section herein, as the initial seeds. Genes that passed the initial screening filter (1) with an absolute expression level >100 in at least one condition; and (2) with >2 fold changes between at least two conditions) were used for further analysis.

Thus, 4,863 genes, with their expression intensity normalized by each gene's mean and standard deviation values across the four sub-populations, were then analyzed by K-means clustering using Minitab data analysis software (Minitab, Inc., State College, Pa.) (FIG. 2). The 4,863 genes were grouped into 100 clusters (K=100) based upon similarities in their gene expression patterns (Table 3), each containing a different number of genes.

The K-means clustering method groups genes together according to the similarity of the various gene expression patterns. The similarity/dissimilarity of the ith and jth genes are given by the Euclidean distance between the two observations:

$${r}_{ik} = \frac{{\sum }_{j = 1}^{n} ( {y}_{ji} - {\overline{y}}_{i} ) ( {y}_{jk} - {\overline{y}}_{k} )}{( n - 1 ) {s}_{i} {s}_{k}} ,$$

This method is designed to group observations into a collection of K clusters. The value of K can be determined either in advance or as a part of the clustering procedure. This algorithm assigns each item to the cluster having the nearest centroid according to the euclidean distance.

The method begins with an initial partition of K clusters, or K initial centroids (seed points). Then it proceeds through the list of genes, assigning a gene to the cluster whose centroid is nearest. Next, it involves recalculation of the centroid for the cluster receiving the new gene and for the cluster losing the gene. The process was repeated until no more reassignment of genes occurs. To eliminate variation within the gene expressions, the genes were normalized (or standardized) prior to clustering.

This method provides for the grouping of associated, similarly functioning genes into gene clusters. Two groups of characterized clusters herein are known as “cumulative clusters” and “representative clusters.” Listed in Table 3 below are clusters known as cumulative clusters. These 100 gene clusters were made with the recited computer program. Similarly, in Table 4 and FIG. 8, representative clusters (i.e., 1 through 8) were also described. Each of these representative clusters contains representative genes that exhibit similar or identical expression patterns.

TABLE 3

| Cluster ID | Mean (HSC) | S.E. Mean (HSC) | Mean (MPP) | S.E. Mean (MPP) | Mean (CLP) | S.E. Mean (CLP) |
|---|---|---|---|---|---|---|
| 1 | 448.369369 | 71.75899208 | 366.4333333 | 60.01488201 | 130.1972973 | 26.51846755 |
| 2 | 248.338148 | 19.98062998 | 224.6555556 | 18.88314956 | 88.19555556 | 11.05239768 |
| 3 | 71.4294574 | 28.58457148 | 336.650969 | 52.2335183 | 114.9639535 | 35.59108305 |
| 4 | 237.377778 | 23.48442702 | 346.9255556 | 37.60970949 | 93.31333333 | 12.9284119 |
| 5 | 114.088141 | 26.28552364 | 256.9926282 | 31.09228068 | 322.7865385 | 36.07733931 |
| 6 | 384.459929 | 37.25715485 | 419.1024823 | 40.06051461 | 101.4553191 | 18.81285968 |
| 7 | 234.074103 | 46.1536519 | 167.5766667 | 40.60195759 | 564.88 | 102.5398899 |
| 8 | 163.837421 | 36.97028916 | 428.2031447 | 105.0076976 | 101.1660377 | 27.15268698 |
| 9 | 403.134333 | 51.13129089 | 419.9643333 | 54.93092859 | 123.196 | 20.58572637 |
| 10 | 393.347024 | 103.7675328 | 380.2565476 | 98.97450547 | 474.7857143 | 123.3186167 |
| 11 | 962.449502 | 266.7171526 | 719.4072139 | 198.6999942 | 417.9253731 | 121.6756873 |
| 12 | 357.946569 | 55.33746123 | 453.4901961 | 65.06149177 | 395.0088235 | 56.81038078 |
| 13 | 184.566667 | 26.13514968 | 212.5143411 | 28.21404584 | 604.8813953 | 64.86053344 |
| 14 | 128.993067 | 15.1980105 | 371.5942667 | 31.5658981 | 346.6064 | 28.84031826 |
| 15 | 628.105556 | 109.5164177 | 453.3111111 | 77.1990812 | 155.1271605 | 32.22296551 |
| 16 | 381.89375 | 80.57129332 | 227.2538194 | 51.094654 | 599.5541667 | 117.022544 |
| 17 | 240.201068 | 33.25453858 | 166.5002137 | 20.17141402 | 844.3987179 | 168.6133667 |
| 18 | 530.625231 | 77.51225999 | 440.9081019 | 65.03506205 | 137.8722222 | 30.23518806 |
| 19 | 1145.20494 | 665.6144793 | 615.6635802 | 389.4444935 | 664.4296296 | 422.8495387 |
| 20 | 573.038498 | 92.60575375 | 339.9420188 | 61.13006507 | 173.1887324 | 38.72661491 |
| 21 | 232.234058 | 32.42901672 | 309.8518116 | 46.95227957 | 130.3173913 | 16.16988652 |
| 22 | 433.324074 | 64.66628409 | 287.837037 | 48.29712466 | 334.9925926 | 52.28002155 |
| 23 | 498.212676 | 99.66738621 | 244.229108 | 49.69863669 | 130.9732394 | 31.89162786 |
| 24 | 386.225 | 90.3722786 | 149.8871212 | 49.06723144 | 355.1590909 | 87.89715844 |
| 25 | 487.064516 | 89.63322237 | 216.2844086 | 32.2333263 | 205.2580645 | 30.20104419 |
| 26 | 891.951852 | 285.91489 | 466.2472222 | 169.4404742 | 586.2888889 | 200.8400236 |
| 27 | 489.601429 | 77.12785123 | 439.8490476 | 69.01183024 | 442.6085714 | 69.19206861 |
| 28 | 297.066667 | 41.57501361 | 92.82156863 | 21.29625645 | 246.1470588 | 33.60110743 |
| 29 | 505.321552 | 99.36891813 | 400.0514368 | 78.29264394 | 287.4637931 | 62.15193684 |
| 30 | 162.740288 | 19.33026395 | 309.1279376 | 28.84849725 | 385.4748201 | 34.79106182 |
| 31 | 300.222436 | 53.78674795 | 104.4589744 | 45.82350908 | 112.9884615 | 46.08655313 |
| 32 | 279.730833 | 38.11767668 | 71.88833333 | 16.7430033 | 144.87 | 23.69863876 |
| 33 | 453.737778 | 88.63285836 | 277.5518519 | 55.70570672 | 105.6377778 | 21.07807379 |
| 34 | 849.241667 | 171.9664638 | 701.5192529 | 143.3742011 | 228.2258621 | 55.80092763 |
| 35 | 589.673214 | 94.44207677 | 268.9464286 | 48.82222768 | 422.6678571 | 69.15293669 |
| 36 | 384.662676 | 54.36726545 | 144.1464789 | 25.15479638 | 82.72394366 | 20.12479511 |
| 37 | 336.783333 | 57.63916858 | 90.12634409 | 32.39737157 | 127.0774194 | 28.95266798 |
| 38 | 141.668304 | 16.13458719 | 242.7983631 | 19.97593269 | 444.2017857 | 34.88448593 |
| 39 | 337.795513 | 43.63204339 | 122.1775641 | 34.81732779 | 150.7615385 | 36.27301225 |
| 40 | 3285.25606 | 988.4652017 | 2104.904545 | 638.2514815 | 1987.890909 | 591.1087871 |
| 41 | 256.618889 | 26.3978332 | 108.3277778 | 13.43742052 | 54.2 | 17.00307342 |
| 42 | 1720.33799 | 438.5484302 | 1153.343382 | 298.7694753 | 878.1823529 | 229.5438306 |
| 43 | 95.5125514 | 19.87738363 | 382.6156379 | 41.47474405 | 268.0938272 | 34.19995746 |
| 44 | 227.061333 | 19.79634178 | 96.81133333 | 18.63043114 | 228.568 | 21.20668033 |
| 45 | 176.704762 | 39.00326262 | 101.8863095 | 31.68220843 | 222.8767857 | 47.81518981 |
| 46 | 894.581548 | 256.1356645 | 367.8714286 | 107.6880431 | 772.05 | 220.0769652 |
| 47 | 464.187654 | 69.71611948 | 591.7685185 | 94.182265 | 335.6740741 | 52.35456113 |
| 48 | 4038.36752 | 999.450683 | 2924.966666 | 734.5688268 | 2769.510256 | 697.7788995 |
| 49 | 44.2356209 | 9.481818286 | 235.7166667 | 17.36974428 | 60.91568627 | 8.975990796 |
| 50 | 338.923134 | 31.17606125 | 402.8803483 | 34.62814513 | 118.5895522 | 15.49197544 |
| 51 | 308.154167 | 71.0637518 | 54.11875 | 25.61163391 | 119.75 | 23.6820097 |
| 52 | 598.726712 | 84.74934241 | 359.7922374 | 50.69874983 | 175.6835616 | 29.71484102 |
| 53 | 405.307895 | 105.1510075 | 540.3787281 | 135.3153465 | 127.7552632 | 21.70818032 |
| 54 | 272.652381 | 32.08903412 | 93.93809524 | 35.34511339 | 156.2 | 30.86864229 |
| 55 | 1171.72051 | 392.3780738 | 696.5673077 | 255.5345525 | 813.2384615 | 292.9672457 |
| 56 | 304.217901 | 51.51391934 | 202.282716 | 38.64246371 | 96.97407407 | 27.17796608 |
| 57 | 374.05641 | 85.49446964 | 117.5647436 | 30.83971667 | 201.7384615 | 45.53796593 |
| 58 | 345.957576 | 69.08672742 | 83.03787879 | 30.49774525 | 230.5 | 50.16892555 |
| 59 | 368.839286 | 111.2935154 | 87.11904762 | 52.16432998 | 184.45 | 63.36841411 |
| 60 | 385.590476 | 83.86591453 | 154.9007937 | 45.4596658 | 442.3666667 | 92.41639267 |
| 61 | 132.826307 | 17.44490209 | 105.0616013 | 19.18017611 | 337.4970588 | 27.65318941 |
| 62 | 856.323214 | 260.7442031 | 450.7964286 | 159.002298 | 372.2678571 | 138.4038474 |
| 63 | 107.154878 | 17.05573901 | 368.454065 | 32.00394251 | 232.6414634 | 22.1677048 |
| 64 | 189.639885 | 19.98760087 | 335.915977 | 27.00201784 | 344.0317241 | 28.12663362 |
| 65 | 157.779915 | 25.91469737 | 217.0313034 | 30.13610496 | 353.4929487 | 41.00862462 |
| 66 | 549.480556 | 128.3040531 | 374.8865079 | 83.39664578 | 166.3714286 | 40.32183324 |
| 67 | 225.061376 | 52.14574297 | 767.2652116 | 158.1627735 | 756.8563492 | 162.3611589 |
| 68 | 477.983951 | 68.89599521 | 301.7098765 | 42.50590991 | 426.4666667 | 59.72299495 |
| 69 | 154.156634 | 14.2347744 | 311.5328479 | 23.32252021 | 195.268932 | 16.42922171 |
| 70 | 215.195 | 31.87292006 | 382.0533333 | 45.78625251 | 108.9525 | 22.4721306 |
| 71 | 152.29058 | 44.24660668 | 362.5155797 | 54.73694545 | 290.3804348 | 51.5229178 |
| 72 | 105.724841 | 23.02416412 | 137.3909766 | 24.26610454 | 604.9522293 | 67.83892292 |
| 73 | 245.044025 | 37.20435029 | 498.5977987 | 73.38648408 | 115.2018868 | 24.46265889 |
| 74 | 130.577729 | 16.81054828 | 336.2861357 | 33.16310399 | 447.3141593 | 40.46352539 |
| 75 | 421.260952 | 104.5705503 | 191.1428571 | 59.96367802 | 130.5314286 | 48.65818486 |
| 76 | 475.328 | 163.2501467 | 187.3366667 | 82.19201921 | 319.324 | 114.1932425 |
| 77 | 1434.4443 | 531.7818093 | 1194.466667 | 446.9158823 | 1021.502632 | 380.0930878 |
| 78 | 318.526042 | 62.41192535 | 91.94895833 | 38.45798861 | 350.06875 | 72.64418176 |
| 79 | 176.45812 | 16.49812767 | 151.1064103 | 16.15094352 | 81.90769231 | 12.50139122 |
| 80 | 280.587778 | 61.23843984 | 90.95555556 | 35.80317268 | 209.0266667 | 49.84729583 |
| 81 | 259.124074 | 24.70749618 | 115.8716049 | 14.83105123 | 35.76296296 | 15.88324939 |
| 82 | 353.495 | 71.57508002 | 90.64583333 | 33.45488541 | 135.29 | 31.77043056 |
| 83 | 790.996465 | 151.6313802 | 446.7161616 | 86.14653795 | 375.6727273 | 77.2104978 |
| 84 | 502.147799 | 89.93245152 | 234.1742138 | 36.21531479 | 108.6792453 | 11.88131876 |
| 85 | 202.159091 | 27.93289851 | 95.18939394 | 21.78176818 | 347.1840909 | 48.05090203 |
| 86 | 218.812255 | 15.72600796 | −19.26372549 | 15.04663684 | −30.76029412 | 13.36115462 |
| 87 | 258.814583 | 26.2046869 | 76.26770833 | 17.21699879 | 180.39375 | 19.26275869 |
| 88 | 279.607778 | 30.40740298 | 88.03444444 | 20.05388386 | 149.72 | 20.94927025 |
| 89 | 817.314431 | 121.2359088 | 837.1373984 | 126.934881 | 327.5219512 | 46.48829629 |
| 90 | 1001.30476 | 234.7393339 | 952.6112245 | 210.8770982 | 655.977551 | 146.0315606 |
| 91 | 160.694805 | 74.4112059 | 317.8950216 | 97.83162379 | 509.7480519 | 154.9252292 |
| 92 | 177.638211 | 12.8465989 | 80.66585366 | 12.21418838 | 106.4097561 | 15.59600981 |
| 93 | 291.496099 | 40.95911575 | 463.4382979 | 61.60463808 | 296.5702128 | 41.69785109 |
| 94 | 294.153704 | 52.5387391 | 330.8551852 | 63.76196922 | 483.8444444 | 79.99064381 |
| 95 | 473.742949 | 56.24688319 | 397.8817308 | 47.33807381 | 136.0711538 | 25.49506212 |
| 96 | 295.971569 | 48.83349476 | 128.6333333 | 30.54305816 | 365.8735294 | 58.22596041 |
| 97 | 259.173077 | 37.72491891 | 59.44615385 | 17.72249084 | 161.8230769 | 24.35557682 |
| 98 | 526.74596 | 117.3129772 | 334.0611111 | 66.74004147 | 610.6575758 | 134.8865275 |
| 99 | 38.207989 | 21.68649369 | 77.04421488 | 20.68904565 | 107.1933884 | 16.56129614 |
| 100 | 360.079825 | 66.16883521 | 204.9846491 | 47.15869098 | 533.2736842 | 95.37155876 |

| | | | Number of Genes |
|---|---|---|---|
| | | | In |
| Cluster ID | Mean (CMP) | S.E. Mean (CMP) | Cluster |
| 1 | 381.0513514 | 60.61087582 | 37 |
| 2 | 285.32 | 23.3711003 | 45 |
| 3 | 88.11627907 | 25.04524108 | 86 |
| 4 | 339.4111111 | 34.45364199 | 45 |
| 5 | 130.9807692 | 24.92059668 | 52 |
| 6 | 359.7574468 | 34.42846258 | 47 |
| 7 | 248.0676923 | 51.43251212 | 65 |
| 8 | 513.8037736 | 151.3182311 | 53 |
| 9 | 201.458 | 27.65786881 | 50 |
| 10 | 146.4607143 | 60.21042056 | 28 |
| 11 | 316.3313433 | 95.06936212 | 67 |
| 12 | 143.3441176 | 26.68703751 | 34 |
| 13 | 386.7775194 | 41.50499303 | 129 |
| 14 | 438.2048 | 37.94664828 | 125 |
| 15 | 221.8592593 | 40.36637298 | 81 |
| 16 | 178.3375 | 39.73554474 | 48 |
| 17 | 132.25 | 19.3673559 | 78 |
| 18 | 367.4347222 | 55.99178005 | 72 |
| 19 | 427.762963 | 283.9371828 | 27 |
| 20 | 239.8830986 | 49.46814441 | 71 |
| 21 | 474.2173913 | 76.18196056 | 46 |
| 22 | 146.6888889 | 30.03658184 | 27 |
| 23 | 129.0126761 | 31.55152019 | 71 |
| 24 | 207.3727273 | 61.71632779 | 22 |
| 25 | 152.3645161 | 24.76281228 | 31 |
| 26 | 359.6666667 | 135.989351 | 18 |
| 27 | 170.3428571 | 33.94026123 | 35 |
| 28 | 196.7470588 | 28.76979129 | 17 |
| 29 | 188.6396552 | 47.15021168 | 58 |
| 30 | 476.5611511 | 42.07732063 | 139 |
| 31 | 202.7461538 | 47.27555076 | 26 |
| 32 | 191.97 | 31.46440771 | 20 |
| 33 | 267.0311111 | 52.33885532 | 45 |
| 34 | 238.8344828 | 50.84490197 | 58 |
| 35 | 172.7821429 | 35.83495126 | 28 |
| 36 | 101.0859155 | 22.36928237 | 71 |
| 37 | 84.86451613 | 29.43606505 | 31 |
| 38 | 428.8982143 | 33.54816334 | 112 |
| 39 | 193.4307692 | 37.07208893 | 26 |
| 40 | 1288.80303 | 383.7007572 | 33 |
| 41 | 195.62 | 18.61673972 | 30 |
| 42 | 614.5352941 | 165.7304504 | 68 |
| 43 | 233.4592593 | 27.30283675 | 81 |
| 44 | 277.14 | 20.45537256 | 25 |
| 45 | 320.6410714 | 63.72266461 | 56 |
| 46 | 258.1678571 | 88.1660424 | 28 |
| 47 | 184.612963 | 33.81060157 | 54 |
| 48 | 1691.846154 | 440.8697087 | 39 |
| 49 | 127.972549 | 9.274926807 | 51 |
| 50 | 251.6895522 | 23.97565148 | 67 |
| 51 | 50.2625 | 25.58960869 | 16 |
| 52 | 172.4342466 | 27.75891325 | 73 |
| 53 | 117.9355263 | 19.69402721 | 76 |
| 54 | 261.7214286 | 32.25918249 | 14 |
| 55 | 452.1730769 | 192.735278 | 26 |
| 56 | 237.5333333 | 41.75893067 | 27 |
| 57 | 129.6192308 | 32.02946481 | 26 |
| 58 | 139.0272727 | 31.93614963 | 11 |
| 59 | 150.1428571 | 55.93588433 | 14 |
| 60 | 290.3238095 | 68.53116875 | 21 |
| 61 | 362.9892157 | 30.27819444 | 102 |
| 62 | 314.3607143 | 125.6865705 | 28 |
| 63 | 357.402439 | 32.82499832 | 82 |
| 64 | 564.8213793 | 42.73954848 | 145 |
| 65 | 492.6916667 | 52.60252269 | 156 |
| 66 | 284.347619 | 63.80985648 | 42 |
| 67 | 587.3880952 | 102.7515614 | 126 |
| 68 | 151.2444444 | 23.99980492 | 27 |
| 69 | 480.0029126 | 39.72414823 | 103 |
| 70 | 240.475 | 32.02751857 | 40 |
| 71 | 110.0521739 | 43.01130598 | 46 |
| 72 | 145.9624204 | 24.13041594 | 157 |
| 73 | 95.03207547 | 21.68366463 | 53 |
| 74 | 401.4619469 | 35.81746588 | 113 |
| 75 | 237.5628571 | 63.69547206 | 35 |
| 76 | 164.292 | 84.45710172 | 25 |
| 77 | 581.8947368 | 238.2954508 | 38 |
| 78 | 301.5625 | 60.823648 | 16 |
| 79 | 281.6128205 | 25.00683528 | 39 |
| 80 | 249.62 | 56.38702671 | 15 |
| 81 | 271.7851852 | 26.14160633 | 27 |
| 82 | 136.88 | 33.64773129 | 20 |
| 83 | 276.3727273 | 56.51908321 | 33 |
| 84 | 207.8490566 | 27.54436988 | 53 |
| 85 | 246.3295455 | 32.13481357 | 44 |
| 86 | 0.710294118 | 11.26354947 | 68 |
| 87 | 178.7875 | 20.98868123 | 16 |
| 88 | 152.4333333 | 20.37282426 | 15 |
| 89 | 244.8231707 | 38.03358657 | 82 |
| 90 | 326.0244898 | 70.59633908 | 49 |
| 91 | 306.9961039 | 100.3444194 | 77 |
| 92 | 267.3243902 | 19.73209246 | 41 |
| 93 | 147.8723404 | 26.8167007 | 47 |
| 94 | 151.4044444 | 34.63235422 | 45 |
| 95 | 250.6692308 | 32.62296549 | 52 |
| 96 | 142.6794118 | 32.53497084 | 34 |
| 97 | 130.4923077 | 23.93208998 | 13 |
| 98 | 208.9666667 | 44.23172253 | 33 |
| 99 | 327.6272727 | 25.35405554 | 121 |
| 100 | 282.1815789 | 50.30196129 | 38 |

TABLE 4
Cluster 1-Genes Down-Regulated in MPP as Compared to HSC

| | | | Fold Changes |
|---|---|---|---|
| Gene Name | GeneBank Reference | Functional Category | MPP vs HSC |
| AML-1 (Cbfa1/Osf2/Runx2) | D14636 | A | −2.4 |
| CCAAT/enhancer binding protein (C/EBP), beta | M61007 | A | −2.6 |
| Gut enriched Kruppel-like factor | U20344 | A | −2.2 |
| Jun-B | U20735 | A | −3.5 |
| Transcription factor LRG-21 | U19118 | A | −2.1 |
| Zinc finger protein 36 | X14578 | A | −2.8 |
| FMS-like tyrosine kinase 3 | X59398 | C | −2.3 |
| Phosphatidylinositol 3-kinase regulatory subunit | AV028190 | C | −4.6 |
| Ly-6E.1 alloantigen (Sca-1) | XD4653 | D | −3.7 |
| N10 a nuclear hormonal binding receptor | X16995 | D | −2 |
| Flamingo 1 | AB028499 | E | −2.4 |
| Notch-1 | AV374287 | E | −5 |
| Cathepsin S | AJ223208 | G | −5 |
| Retinol binding protein 1, cellular | X60367 | I | −2.1 |
| Fibroblast growth factor 4 (FGF-4) | X14849 | J | −2.2 |
| Small inducible cytokine A3 | J04491 | J | −2.2 |
| TGF beta-induced protein, 68 kDa | L19932 | J | −2.4 |
| Protein tyrosine phosphatase (PAC-1) | U09268 | K | −2.2 |

Cluster 2-Genes Up-Regulated in MPP as Compared to HSC

| | | | Fold Changes |
|---|---|---|---|
| Gene Name | GeneBank Reference | Functional Category | MPP vs HSC |
| Hepatic nuclear factor 6 (HNF-6) | U95945 | A | 2.3 |
| CDC28 protein kinase | AA681998 | C | 3.3 |
| Janus kinase 2 (JAK2) | L16956 | C | 2.1 |
| Serine/threonine kinase (nek2) | AF013166 | C | 3.2 |
| Serine/threonine kinase (sak-b) | L29480 | C | 3.1 |
| Serine/threonine kinase 6 | U80932 | C | 3.3 |
| CD48 antigen | X53526 | D | 3.8 |
| Male enhanced antigen 1 | D17341 | D | 2.1 |
| Acetyltransferase Tubedown-1 | A1645561 | E | 2.6 |
| Bone morphogenetic protein BMP-4 | L47480 | E | 2 |
| Wnt10a | U61969 | E | 2.4 |
| Caspase-3 | U54803 | G | 2.4 |
| Cathepsin G | X70057 | G | 2.6 |
| Heat shock protein (Hsp70) | U08215 | G | 2.3 |
| T-IAP (Inhibitor of Apoptosis) | AB013819 | G | 3.6 |
| Budding inhibited by benzimidazoles 1 (BUB1) homlg | AF002823 | H | 2.0 |
| Cell division cycle control protein 2a | M38724 | H | 6.2 |
| Cyclin A2 | X75483 | H | 6.7 |
| Cyclin B1 | X64713 | H | 2.4 |
| Cyclin B2 | X66032 | H | 2.2 |
| Cyclin F | Z47766 | H | 2.5 |
| Kinesin-related mitotic motor protein | AJ223293 | H | 4.0 |
| Mitotic centromere-associated kinesin | AA007891 | H | 2.2 |
| Mitotic checkpoint component Mad2 | U83902 | H | 2.9 |
| Mitotic checkpoint protein kinase BUB1B (Bub1b) | AW049504 | H | 4.9 |
| Rab6, kinesin-like (Rab6kil) | AV059766 | H | 4.4 |
| Replication factor C 4 (RFC4) | AW122092 | H | 3.0 |
| Telomeric repeat binding factor 2 (TERF-2) | AW122405 | H | 2.0 |
| Chemokine (C-C) receptor 1-like | U28405 | J | 2.1 |
| Cytokine receptor-like factor 1 (Crlf1) | AA270365 | J | 2.4 |
| Fibroblast growth factor 2 (FGF-2) | AF065903 | J | 2 |
| Small inducible cytokine A9 | U49513 | J | 2.4 |
| DDX3 (Putative RNA helicase) | AI047912 | N | 2.2 |
| Eukaryotic translation initiation factor 4B (elF4B) | AW121930 | N | 2.2 |
| U5 small nuclear ribonucleoprotein (Snrp116) | U97079 | N | 2.8 |
| Karyopherin (importin) alpha 2 | D55720 | O | 3.5 |
| High mobility group protein homolog HMG4 (Hmg4) | AF022465 | P | 2.2 |

Cluster 3-Genes Down-Regulated in Both CLP and CMP as Compared to MPP

| | | | Fold Changes | |
|---|---|---|---|---|
| Gene Name | GeneBank Reference | Functional Category | CLP vs MPP | CMP vs MPP |
| Activating transcription factor 4 (ATF-4) | M94087 | A | −2.3 | −2.8 |
| Core promoter element binding protein (COPEB) | AI846501 | A | −16.8 | −17.9 |
| Early growth response 1 (egr1) | M28845 | A | −2.4 | −2.2 |
| Jun-D | J04509 | A | −4.4 | −7.5 |
| Transcription factor LRG-21 | U19118 | A | −6.3 | −12.2 |
| Zinc finger protein 36 | X14678 | A | −3.2 | −3.9 |
| G protein signaling regulator RGS2 (rgs2) | U67187 | C | −3.3 | −2.4 |
| Male enhanced antigen 1 | D17341 | D | −2.2 | −2.3 |
| Bone morphogenetic protein BMP-4 | L47480 | E | −2.2 | −2.9 |
| RhoB | X99963 | G | −2.5 | −2.3 |
| Secretory leukocyte protease inhibitor (Slpi) | AV093322 | G | −2.2 | −2.8 |
| Fibroblast growth factor 2 (FGF-2) | AF065903 | J | −2.9 | −2 |
| EEF-Tu encoding elongation factor Tu | M17878 | N | −2.4 | −4.2 |
| Eosinophil 2ndary granule ribonuclease-1 (mEAR-1 | U72032 | N | −12.6 | −11.6 |
| Eosinophil-associated ribonuclease 3 (Ear3) | AF017258 | N | −7.1 | −11.5 |

Cluster 4-Genes Up-Regulated in Both CLP and CMP as Compared to MPP

| | | | Fold Changes | |
|---|---|---|---|---|
| Gene Name | GeneBank Reference | Functional Category | CLP vs MPP | CMP vs MPP |
| 11-zinc-finger transcription ffactor (CTCF) | U51037 | A | 6.3 | 6.3 |
| Bromodomain adjacent to zinc finger domain, 2A | AW122821 | A | 4.6 | 4.1 |
| CDC45-related protein (Cdc45) | AF098068 | A | 4.4 | 6.2 |
| Core binding factor alpha 1 (Cbfa1/Osf2/Runx2) | D14636 | A | 5.9 | 3.4 |
| Forkhead box M1 (Foxm1) | Y11245 | A | 3.5 | 4.0 |
| Ikaros DNA binding protein | L03547 | A | 4.9 | 2.1 |
| LIM-only domain transcription factor LMO-4 | AF074600 | A | 4.9 | 6.5 |
| Max-interacting transcriptional repressor (Mad4) | U32395 | A | 2.5 | 2 |
| N-Myc interactor (Nmi) | AF019249 | A | 2.1 | 2.0 |
| Nuclear transcription factor RelA (Rela) | AI845667 | A | 3.1 | 3.5 |
| pOU domain, class 2, transcription factor 1 (Oct-1B) | X68363 | A | 2.4 | 2.9 |
| Taube nuss | AW214244 | A | 3.3 | 3.4 |
| Zinc finger protein 265 (Zip265) | AI835041 | A | 3.7 | 4.8 |
| Janus kinase 3 (JAK 3) | L40172 | C | 2.2 | 4.5 |
| Protein kinase Chk2 (Chk2) | AF086905 | C | 2.3 | 3.3 |
| RAN GTPase activating protein 1 | U20857 | C | 2.9 | 3.5 |
| Ras-related protein (Krev-1) | AW049685 | C | 2.3 | 2.3 |
| Serine-threonine protein kinase (MNBH) | AW124678 | C | 3.2 | 3.1 |
| Smad4 | U79748 | C | 4.1 | 4.4 |
| STATG | L47650 | C | 3.2 | 3.9 |
| CD164 (MGC-24v) | AB014464 | D | 2 | 2 |
| CD1d1 antigen | M63695 | D | 2.4 | 2.8 |
| CD44 antigen | X66084 | D | 2.8 | 2.1 |
| Fibronectin receptor beta-chain (VLA5-homolog) | X15202 | D | 2.1 | 3.6 |
| Inositol trisphosphate receptor type 2 (Itpr2) | AF031127 | D | 4.4 | 2.1 |
| Lymphocyte antigen 86 (Ly86) | AB007599 | D | 9.4 | 4.1 |
| Steroid receptor RNA activator 1 (Sra1) | AW122167 | D | 2.5 | 2.7 |
| Granule cell differentiation protein (Gcdp) | D78188 | E | 2.2 | 2.6 |
| Notch-1 | AV374287 | E | 14.2 | 7.0 |
| Apoptosis-related RNA binding protein (Napor-3) | AW061318 | G | 3.1 | 3.5 |
| BH3 interacting domain death agonist | U75506 | G | 2.6 | 3.2 |
| Caspase-3 | U54803 | G | 3.8 | 4.4 |
| Caspase-6 | Y13087 | G | 4.2 | 2.9 |
| Caspase-8 | AJ007749 | G | 5.5 | 4.5 |
| Catalase | M29394 | G | 2.9 | 3.2 |
| Hypoxia inducible factor 1, alpha subunit | F003695 | G | 2.6 | 2.2 |
| Cyclin-dependent kinase homologue (p130PITSL) | L37092 | H | 2.0 | 2.0 |
| mutant p53 | AB021961 | H | 3.1 | 5.2 |
| Myb proto-oncogene | M12848 | H | 2.1 | 2.5 |
| Myelocytomatosis oncogene | L00039 | H | 2.4 | 3 |
| RAB9, member RAS oncogene family (Rab9) | AB027290 | H | 3.1 | 5.8 |
| Relinoblastoma-like 1 (p107) | U27177 | H | 2.4 | 2.4 |
| Lcr-1 (CXCR-4 homologue) | Z80112 | I | 4.6 | 3.9 |
| Stromal cell derived factor receptor 1 (Sdfrt) | D50463 | I | 2.5 | 3.1 |
| Interferon (alpha and beta) receptor | M89641 | J | 4.4 | 3.9 |
| Phosphatase and tensin homolog (PTEN) | U92437 | K | 7.6 | 3.4 |

Cluster 5-Genes Down-Regulated in CLP and CMP as Compared to CMP and MPP

| | | | Fold Changes | |
|---|---|---|---|---|
| Gene Name | GeneBank Reference | Functional Category | CLP vs CMP | CLP vs MPP |
| Friend of GATA-1 (FOG) | AF006492 | A | −10.8 | −7.2 |
| GATA-binding protein 1 | X15763 | A | −8.6 | −2.0 |
| GATA-binding protein 2 | AB000096 | A | −22.6 | −9.8 |
| LIM-only domain transcription factor LMO-2 | M64360 | A | −3.4 | −3.6 |
| Apolipoprotein E | D00466 | B | −76.6 | −13.5 |
| G-protein coupled thrombin receptor | AW123850 | D | −3.5 | −5.7 |
| Transferrin receptor 2 (Trfr2) | AI596094 | D | −3.7 | −4.0 |
| Tyrosine kinase receptor 1 (Tie1) | AV235418 | D | −9.1 | −2.6 |
| Cathepsin G | X70057 | G | −46 | −6 |
| Myeloperoxidase | X15313 | G | −11.4 | −4.7 |
| Proteinase 3 | U43525 | G | −17.8 | −7.6 |
| Spi2 proteinase inhibitor (spi2/eb1) | M64085 | G | −38.4 | −25.0 |
| Retinol binding protein 1, cellular | X60367 | I | −7.2 | −5.0 |
| Small inducible cytokine A9 | U49513 | J | −26.6 | −10.2 |
| Neutrophil elastase | UD4962 | K | −11.5 | −3.5 |

Cluster 6-Genes Up-Regulated in CLP as Compared to CMP and MPP

| | | | Fold Changes | |
|---|---|---|---|---|
| Gene Name | GeneBank Reference | Functional Category | CLP vs CMP | CLP vs MPP |
| Basic-helix-loop-helix protein (bHLH) | Y07836 | A | 3.3 | 2.1 |
| B-cell-specific coactivator BOB.1/OBF.1 | Z54283 | A | 8.1 | 3.3 |
| AML-1 (Cbfa1/Osf2/Runx2) | D14636 | A | 2.1 | 5.9 |
| Ikaros DNA binding protein | L03547 | A | 2.4 | 4.9 |
| Inhibitor of DNA binding 2 (ID-2) | AF077861 | A | 30.9 | 9.3 |
| T-cell specific transcription factor 7 (Tcf7) | AI019193 | A | 12.6 | 4.7 |
| Tubby super-family protein (Tusp) | AI848591 | A | 8.1 | 2.8 |
| Cytochrome c oxidase subunit V1aH | U08439 | B | 108.1 | 26.4 |
| B lymphoid kinase | M30903 | C | 13.3 | 4.6 |
| FMS-like tyrosine kinase 3 | X59398 | C | 4.1 | 2.8 |
| Intracellular calcium-binding protein (MRP14) | M83219 | C | 31.7 | 4.6 |
| Intracellular calcium-binding protein (MRP8) | M83218 | C | 31.4 | 3.2 |
| LIM-kinase 1 (Limk1) | AW125574 | C | 2 | 2.2 |
| Rearr. lymphocyte protein-tyrosine kinase (LCK) | M12056 | C | 9.7 | 9.5 |
| Smad7 | AF015260 | C | 3.5 | 3.2 |
| B cell linker protein BLNK | AF068182 | D | 78.3 | 22.1 |
| Interleukin 7 receptor | M29697 | D | 36.8 | 16.1 |
| Interleukin-18 receptor accessory protein-like | AF077347 | D | 6.5 | 4.2 |
| Lymphocyte antigen 6 complex, locus D | X63782 | D | 125.3 | 44.7 |
| NK (natural killer) cell receptor 2B4 (CD244) | L19057 | D | 2.2 | 2.3 |
| Putative transmembrane receptor IL-1Rrp | U43673 | D | 9.9 | 3.6 |
| Hairy and enhancer of split 6 | AW048812 | E | 2.4 | 2.3 |
| Notch-1 | AV374287 | E | 26.6 | 14.2 |
| Cathepsin S | AJ223208 | G | 2.9 | 4.4 |
| Pre-B lymphocyte 1 (Vpreb1) | AV015107 | I | 36.6 | 13.5 |
| Recombination activating 1 (Rag-1) | M29475 | I | 43.2 | 13.4 |
| Recombination activating 2 (Rag-2) | M64796 | I | 11.1 | 4.2 |
| T-cell receptor beta-chain constant region | M26056 | I | 9.1 | 4.8 |
| Terminal deoxynucleotidyl transferase (Tdt) | AV312871 | I | 21.6 | 31.5 |
| Interleukin 12a | M86672 | J | 6.2 | 7.6 |
| Lymphotoxin-beta | U16985 | J | 5.9 | 6.2 |
| Small inducible cytokine A5 | AF065947 | J | 13.2 | 2.1 |

Cluster 7-Genes Down-Regulated in CMP as Compared to CLP and MPP

| | | | Fold Changes | |
|---|---|---|---|---|
| Gene Name | GeneBank Reference | Functional Category | CMP vs CLP | CMP vs MPP |
| Gul enriched Kruppel-like factor | U20344 | A | −2.3 | −4.8 |
| TGF-beta-stimulated clone-22 (TSC-22) | X62940 | A | −2.6 | −3.6 |
| Cytohesin binding protein (Cbp) | AI120844 | C | −2.5 | −2.6 |
| Intracellular calcium-binding protein (MRP14) | M83219 | C | −31.7 | −2.9 |
| Gamma-aminobutyric acid (GABA-A) receptor | X59300 | D | −2.1 | −2.5 |
| N10 a nuclear hormonal binding receptor | X16995 | D | −2.5 | −2.8 |
| Frizzled homolog 8 (Drosophila) | U43321 | E | −2.7 | −2.2 |

Cluster 8-Genes Up-Regulated in CMP as Compared to CLP and MPP

| | | | Fold Changes | |
|---|---|---|---|---|
| Gene Name | GeneBank Reference | Functional Category | CMP vs CLP | CMP vs MPP |
| cAMP response element binding protein (CREB) | U46026 | A | 2.1 | 3.4 |
| CCAAT/enhancer binding protein (C/EBP), delta | X61800 | A | 6 | 3.2 |
| Double LIM protein-1 | D88792 | A | 4.2 | 2.2 |
| Erythrocyte protein band 4.1 | L00919 | A | 7.6 | 2.9 |
| Metallotihionein 1 | V00835 | A | 10.8 | 3.8 |
| Apolipoprotein E | D00466 | B | 76.6 | 2.4 |
| Histidine decarboxylase cluster | X57437 | B | 12.3 | 3.1 |
| (TNFRSF)-interacting serine-threonine kinase 1 | U25995 | C | 3.2 | 2.2 |
| CD9 antigen | L08115 | D | 7.9 | 2.4 |
| c-kit | Y00864 | D | 4 | 2 |
| Colony stimulating factor 1 receptor | X06368 | D | 2 | 2.3 |
| Granulocyte colony-stimulating factor receptor | M58288 | D | 23.7 | 4.6 |
| MCP-1 Receptor | U56819 | D | 2.2 | 3.3 |
| TNF receptor superfamily, member 1a | X57796 | D | 6.9 | 3.9 |
| Cathepsin G | X70057 | G | 46 | 5.3 |
| Cathepsin Z precursor (clsZ) | AJ242663 | G | 2.9 | 3 |
| Granzyme F | AI504305 | G | 3.9 | 6.9 |
| Myeloperoxidase | X15313 | G | 11.4 | 2.2 |
| c-fes proto-oncogene | X12616 | H | 4.3 | 4.1 |
| Beta Fc receptor type II (FCRII) | M31312 | I | 9.0 | 3.1 |
| Small inducible cytokine A9 | U49513 | J | 26.6 | 2.6 |
| Transforming growth factor-beta 1 | AJ009862 | J | 4.9 | 6.2 |
| GTP-binding protein (Rab3D) | M89777 | O | 11.7 | 4.9 |
| Histone H1x | AI851599 | P | 10.5 | 2.9 |
| Rad1 DNA damage checkpoint protein | AF073523 | P | 5.6 | 3.5 |

### Example 13

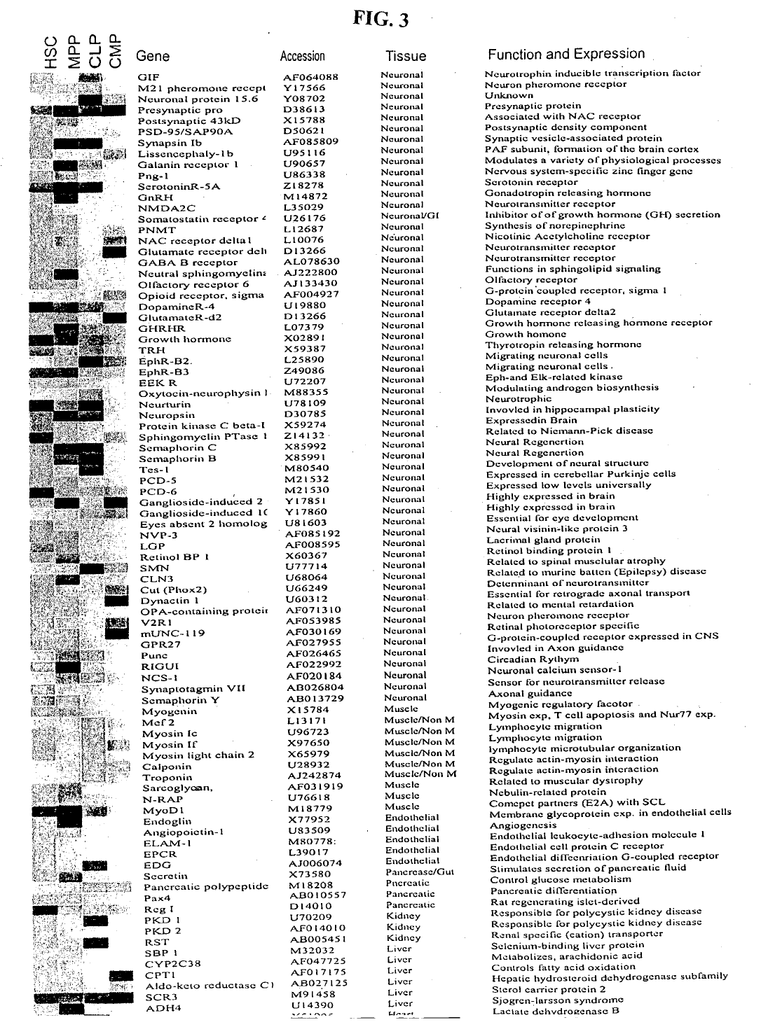

The data of Table 1 were processed using the K-means clustering. Transcripts of a variety of non-hematopoietic genes were detected in cells undergoing early hematopoiesis (Tables 1, 2). HSCs expressed 43 of 58 genes specific to non-hematopoietic tissues, detected by chip hybridization. These non-hematopoietic tissues included brain, liver, heart, kidney, pancreas, muscle, and endothelium, as listed in FIG. 3. Expression of the majority of these non-hematopoietic genes was progressively attenuated in MPPs and downstream CMPs and CLPs. Thus, promiscuous expression of non-hematopoictic genes (i.e., non-hematopoietic promiscuity) was most pronounced in the HSC population (Tables 1, 2).

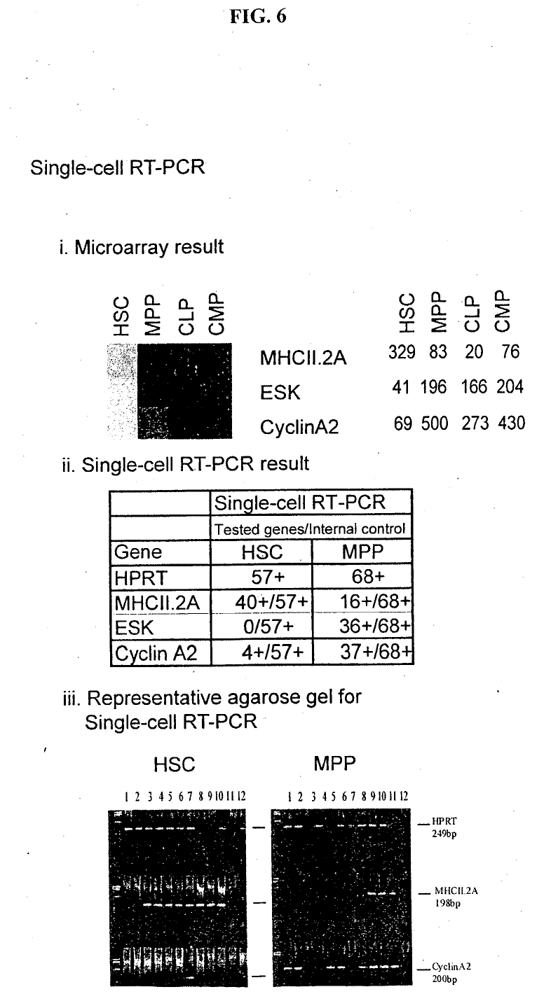

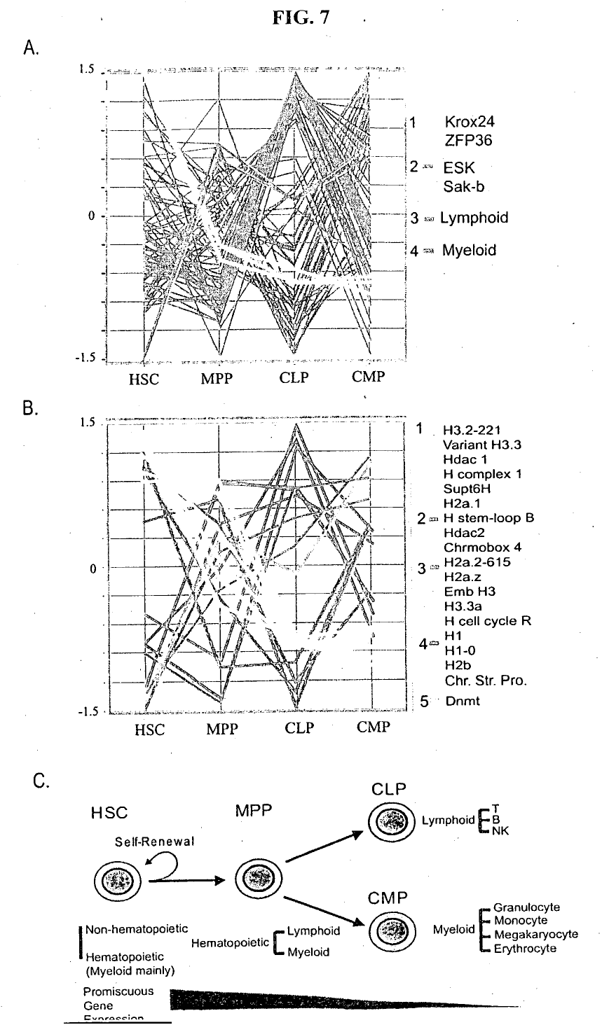

To exclude the possibility that the non-hematopoietic gene RNA transcripts were derived from bone marrow non-hematopoietic cells sharing the same phenotype as HSCs, a hematopoietic cell population was purified using CD45, a hematopoiesis-specific marker. cRNA was amplified from highly purified long-term HSCs of Lin⁻CD34^{-/lo}c-Kit⁺Sca-1⁺CD45⁺ phenotype (FIGS. 1A, 6). The nucleic acids were again hybridized to the MGU-74A chip, resulting in a similar expression pattern of hematopoietic and non-hematopoietic genes.

Four genes were randomly chosen (SBP-1, GnRH, N-RAP, and Phox2) from the list of expressed genes. These genes were tested for expression by RT-PCR targeting for 1 and 10 cells of CD45+HSCs. SBP-1 is a selenium-binding liver protein; GnRH regulates the production of testosterone via the hypothalamic-pituitary-gonadal axis; N-RAP encodes a Nebulin-related protein and is specifically expressed in skeletal and cardiac muscle; and Phox2 is required for induction of expression of pan-neuronal genes, including tyrosine hydroxylase (TH). As shown in FIG. 3C, these genes were detectable at single or 10 cell levels. Differences in cell numbers required for positive detection in RT-PCR analyses might represent the frequency of cells expressing these target genes, the difference in copy numbers of transcripts per cell, or both. Thus, it was determined that it is likely that a majority of non-hematopoietic genes detected in the Affymetrix chip are expressed in a significant population of CD45⁺ HSCs. This data is useful in mapping non-hematopoietic genes.

### Example 14

Single-cell RT-PCR was used to confirm the array results of Example 6 for several representative genes.

Single cell RT-PCR was carried out according to a previous report with slight modifications, including 1) single cells of HSC and MPP were directly triple-sorted into 96-well arrays of 0.2 ml microamp tubes; and, 2) the lysis buffer contained 0.5% Triton® X-100 (Sigma-Aldrich Corp., St. Louis, Mo.), instead of 0.4% NP-40. Nested primers for each gene were used for the second round PCR. Primers used for RT-nested-PCR are listed as follows: HPRT: SEQ ID NO 4864 F1, 5′GGGGGCTATAAGTTCTTTGC3′ and SEQ ID NO 4865 R1, 5′TCCAACACTTCGAGAGGTCC3′; SEQ ID NO 4866 F2, 5′GTTCTTTGCTGACCTGCTGGC′ and SEQ ID NO 4867 R2, 5′TGGGGCTGTACTGCTTAACC3′. MHCII.2A: SEQ ID NO 4868 F1, 5′CCCATGTCAGAGCTGACAGAGA3′ and SEQ ID NO 4869 R1, 5′CAAGGGAAAAGCAAGTTG3′; SEQ ID NO 4870 F2, 5′ATCGTGGTGGGCACCATC3′ and SEQ ID NO 4871 R2, 5′TGGGGGTCACTTGAAGAAG3′. ESK; SEQ ID NO 4872 F1, 5′CTTGGCTTTCAGAGACGA3′ and SEQ ID NO 4873 R1, 5′TGACTATACCGACCAATC3′; SEQ ID NO 4874 F2, 5′ATTTAGAAATGGAGGCT3′ and SEQ ID NO 4875 R2, 5′AATTCAACCAGTTCTCTGGG3′. CyclinA2: SEQ ID NO 4876 F1, 5′AAATGTAAACCTAAAGTGGG3′ and SEQ ID NO 4877 R1, 5′AAATGTAAACCTAAAGTGGG3′; SEQ ID NO 4878 F2, 5′CATGAAGAGGCAACCAGACA3′ and SEQ ID NO 4879 R2, 5′CGAAGCTAGCAGCATAGCAG3′.

The relative expression levels of lineage-affiliated genes such as G-CSFR, C/EBPα, GATA-1, and λ5, in each sub-population correlated with those estimated by semi-quantitative RT-PCRs. Furthermore, single cell RT-PCR analyses of MHCII.2A, ESK and Cyclin A2 (representative of genes from HSCs and MPPs) revealed that the relative quantity of gene expression levels detected on the oligonucleotide array correlated with percentages of cells that express the corresponding genes (FIG. 6).

### Example 15

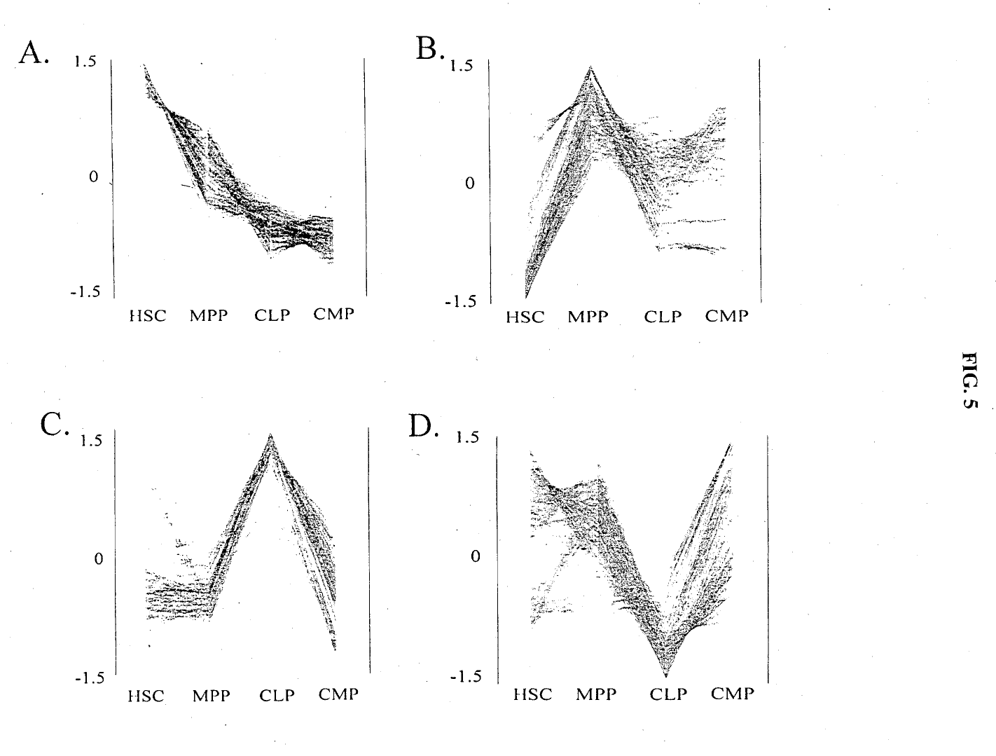

The expression patterns displayed by the K-means clustering method of Example 12 were converted into illustrations using Spotfire® software. Genes were classified that were predominantly expressed in the targeted four sub-populations of hematopoietic cells into gene clusters shown in FIG. 5A through 5D.

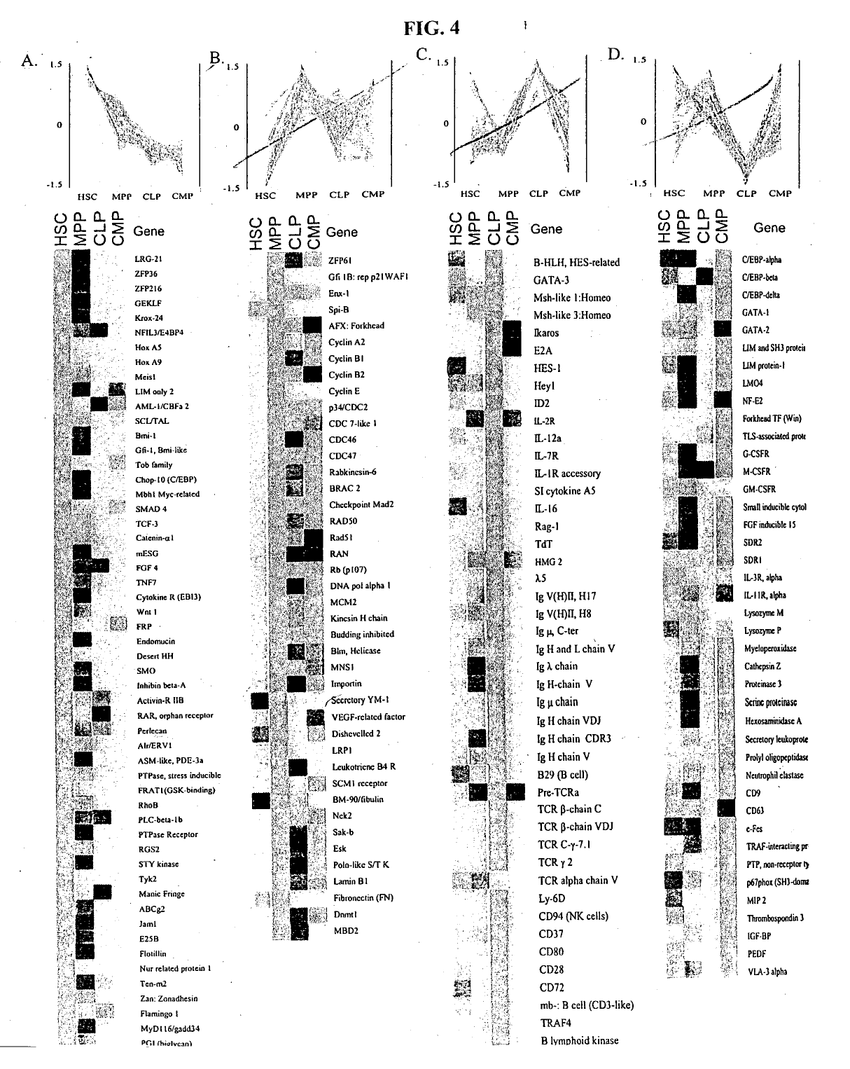

The clusters were illustrated using Eisen's software package to analyze the normalized data from the 4,863 genes to obtain a global view of their expression patterns, which is shown in FIGS. 2, 3, and 4. The pattern of the genes in each cluster can be clearly viewed through the normalized genes in each cluster. The genes up-regulated in HSC (SEQ ID NOs 3428-4863) initially are shown to down-regulate as lineage commitment occurs. FIGS. 4B-4D show genes up-regulated in MPP (SEQ. ID NOs, 2076-3427), FIG. 4A shows CLP (SEQ. ID NOs. 1-821), and CMP (SEQ. ID NOs. 822-2075), respectively. Listed in Tables 3 and 4 are several clusters of known genes based on their gene expression patterns during the progression of HSC proliferation and differentiation.

In conclusion, a method which correlates gene expression with lineage development was shown. In particular, gene intensity was shown to vary according to a given cell stage. As such, this provides an illustration of the mode by which genes are up- and down-regulated during cell lineage commitment.

### Example 16

The data of Table 2 is further analyzed and explained. Transcripts of a variety of non-hematopoietic genes were detected in cells undergoing early hematopoiesis (Table 2). HSCs expressed 43 of 58 genes specific to non-hematopoietic tissues detected by chip hybridization. These non-hematopoietic tissues included brain, liver, heart, kidney, pancreas, muscle, and endothelium, as listed in FIG. 3. Expression of the majority of these non-hematopoietic genes was progressively attenuated in MPPs and downstream CMPs and CLPs. Thus, promiscuous expression of non-hematopoietic genes (i.e., non-hematopoietic promiscuity) was most pronounced in the HSC population (Table 2).

To exclude the possibility that the non-hematopoietic gene transcripts may be derived from bone marrow non-hematopoietic cells sharing the phenotype with HSCs, hematopoietic cell sub-populations were purified using CD45, a hematopoiesis-specific marker. cRNA was amplified from highly purified long-term HSCs of Lin⁻CD34^{-/lo}c-Kit⁺Sca-1⁺CD45⁺ phenotype (FIG. 1). The nucleic acids were again hybridized to the MGU-74A chip, resulting in a similar expression pattern of hematopoietic and non-hematopoietic genes. Four genes were randomly chosen (SBP-1, GnRH, N-RAP, and Phox2) from the list of expressed genes. These genes were tested for expression by RT-PCR targeting for 1 and 10 cells of CD45+HSCs. SBP-1 is a selenium-binding liver protein; GnRH regulates the production of testosterone via the hypothalamic-pituitary-gonadal axis; N-RAP encodes a Nebulin-related protein and is specifically expressed in skeletal and cardiac muscle; and Phox2 is required for induction of expression of panneuronal genes, including tyrosine hydroxylase (TH).

These four genes were detectable at single or 10 cell levels. Differences in cell numbers required for positive detection in RT-PCR analyses might represent the frequency of cells expressing these target genes, the difference in copy numbers of transcripts per cell, or both. Thus, it was determined that it is likely that a majority of non-hematopoietic genes detected in the Affymetrix chip are expressed in a significant population of CD45⁺ HSCs. This data is useful in mapping non-hematopoietic genes.

### Example 17

From the clustering data, it was determined that hematopoiesis-affiliated genes on a chip contained 160 lymphoid-, 117 myeloid-, and some stern/progenitor-related genes. A partial list of these genes is shown in FIG. 4. HSCs expressed more than 40% of the hematopoiesis-related genes. Interestingly, HSCs expressed GM- and MegE-affiliated genes, including myeloid cytokine receptors and transcription factors, but only a limited number of lymphoid genes. In contrast, MPPs expressed about 30% of hematopoietic genes related to both lymphoid (T and B) and myeloid (GM and Meg E) lineages. CMPs expressed 26% of myeloid (GM- and MegE-affiliated) genes, but not lymphoid genes, whereas CLPs expressed 45% of lymphoid (T-, B-, and NK-affiliated) genes, but not myeloid genes. Hence, co-expression of myeloerythroid genes (myeloid promiscuity) was observed to exist in HSCs, MPPs, and CMPs, whereas co-expression of T/B/NK lymphoid genes (lymphoid promiscuity) existed mainly in MPPs and CLPs. This data strongly suggest that myeloid and lymphoid promiscuity is distributed in a hierarchical and asymmetrical fashion during hematopoietic development and, therefore, the expression of lineage-related genes can precede commitment (FIGS. 2, 3, 4, and 5).

### Example 18

Because groups of genes with similar expression behavior (up-regulation or down-regulation under the same condition) are likely to be functionally related, the relative expression patters of genes were compared within these populations. Among a variety of clustering methods [including self-organization maps (SOMs) and hierarchical clustering], K-means clustering, which uses genes with known functions as initial seeds for clusters, was determined to be most appropriate. This was described in Example 12. There were 137 known genes picked, the biologic functions of which have been well characterized, as the initial seeds. A total of 4,863 genes that passed the initial screening filter were subjected to further analysis. The expression levels of these genes were first standardized (or normalized) and then analyzed by K-means clustering using Minitab data analysis software. The final partition of the 4,863 genes/ESTs resulted in 100 clusters, shown in Table 3, each containing a different number of genes. Genes that were dominantly expressed in each population were the primary focus, grouping them into four sub-population categories (FIG. 4, Table 1).

The clustering analysis revealed again that the majority of nonhematopoiesis-affiliated genes fell into the group listed in FIG. 4A. The group of FIG. 4A also contained genes that might play a role in the regulation of stem cell properties, such as self-renewal. These include Wnt1, desert hedgehog (DHH), TCF3 (a target of Wnt signaling), and Smoothened (SMO, a coreceptor of DHH), which are potentially involved in maintaining stem cell compartments. Genes related to cell growth arrest (e.g., gut-enriched Kruppel-like factor and ZFP36), immortalization of cells (e.g., Bmi-1, a polycomb-group protein), leukemogenesis (e.g. HoxA9 and Meis1), and commitment (e.g. Manic Fringe [Notch activity regulator]) were also found in this category.

It was found that 13.8% of the genes (n=4,863) were significantly up-regulated in MPPs but maintained at various levels in CLPs and CMPs (genes of FIG. 4B). These included 26% of hematopoietic (both myeloid and lymphoid) genes, which were elevated at the MPP stage. Thus, MPPs co-express genes related to multiple myeloid and lymphoid lineages (FIGS. 4C-D), suggesting that both myeloid and lymphoid promiscuity may operate at this stage. Other known genes in this category include regulatory molecules of cell cycling, such as cyclins, CDC molecules, and cell cycle checkpoint molecules (BRCA, MAD2, etc.). Several kinases related to cell proliferation, such as Nek2, Sak-b (a homolog to Drosophila Polo) and Esk were also found in this category. This data is compatible with the fact that MPPs are highly proliferative cells (FIG. 1B) and suggest that MPPs are at a priming stage for both myeloid and lymphoid differentiation.

The majority of genes preferentially expressed in CLPs (41% of hematopoietic-related genes in FIG. 4C) and CMPs (25% of hematopoietic-related genes, genes in FIG. 4D) were lymphoid and myeloid genes, respectively (Table 2, FIG. 4). Genes in FIG. 4C included B, T, and NK lymphoid-associated genes (i.e., E2A, Ikaros, HES-1, Notch1, GATA-3, BLNK, TCRβ, TCRγ, CD94, TdT, RAG-1, B lymphoid kinase, Lck, and IL-7R), whereas genes in FIG. 4D included granulocyte/monocyte- and megakaryocyte/erhthrocyte-affiliated genes (i.e., GATA-1, C/EBPα, β, and δ, LMO4, FOG, and IL-11R, G-CSFR, GM-CSFR). Interestingly, the majority of the genes categorized in categories C and D are likely to be reciprocally regulated between CLPs and CMPs, representing the myeloid-versus-lymphoid branch point. This result suggests that transcriptional regulation of lymphoid-affiliated (T, B, and NK lineages) or myeloid-affiliated (MegE and GM lineages) genes is a mutually exclusive event in the progression from MPPs to either CLPs or CMPs.

In addition to mutually exclusive regulation in the expression of lymphoid-versus myeloid-related genes, a number of genes were up-regulated at the CLP (FIG. 4C) or CMP stage (FIG. 4D) as a result of transition from the MPP stage. These genes encode molecules related to cell differentiation and functions, such as lymphoid-related Lck, λ5, TdT, RAG-1, and myeloid-related LIM and SH3 protein 1, LMO4, SDR1, macrophage inflammatory protein (MIP), and small inducible cytokine A9. This indicated that up-regulation of lineage-affiliated genes was also required for lineage specification.

A method for associating a gene with a stem cell sub-population can be practiced. The method is initiated by isolating a population of bone marrow stem cells or the populations that have more than one-cell population. The population is separated into sub-populations. The RNA from each said sub-population is then isolated. A clonogenic library is formed from the RNA. The library is directed to genes expressed in the sub-population.

The library is then amplified. Gene expression patterns for each sub-population of cells are analyzed with a bioinformatics program by hybridizing with the clonogenic library labeled ESTs. Expression is indicated if an EST attaches to a member of the library. Expressed genes are associated with the sub-populations to build a gene expression map. Next, a gene of unknown function is isolated, with the gene's expression in each sub-population determined. This information is compared with said expression patterns from the sub-population. Based on when the gene is expressed, it is associated with a particular sub-population, and the gene's function can be predicted.

### Example 20

A method for determining a cell sub-population comprising: isolating a population of hematopoietic stem cells; separating said population of stem cells into sub-populations; isolating RNA from each said sub-population; forming a clonogenic library from said RNA directed to genes expressed in said sub-population for each said sub-population and amplifying said library; analyzing gene expression patterns of each said sub-population of cells with a bioinformatics program by hybridizing with said clonogenic library, labeled ESTs, whereby expression is indicated if an EST attaches to a member of said library; associating expressed genes with the sub-populations to build a gene expression map; isolating a cell of unknown function; analyzing said cell's gene expression with said bioinformatics program to determine the gene expression pattern; comparing said unknown cell with said expression patterns from said sub-populations; and, based on said cell's expressed genes, associating said cell with a particular sub-population.

### Example 21

A method is described wherein cell stage commitment from an unknown multilineage-affiliated cell can be identified and characterized. The method is initiated by isolating and amplifying the nucleic acid from an unknown cell.

An unknown hematopoietic cell is isolated as provided in Example 1. Approximately equal amounts of biotinylated cRNA derived from replicate wells of each unknown cell, HSC, MPP, CMP, and CLP cell sub-population may be mixed with antisense biotinylated control cRNA (bioB, bioC, bioD, and cre) and then individually hybridized with the gene matrix, and incubated with strepavidin-conjugated phycoerythrin (PE) complex to detect the hybridization signal intensity, which is an indicator of gene expression level for each corresponding gene on the various gene expression maps.

Replicate hybridization results have been obtained for HSCs, MPPs, CMPs, and CLP sub-populations and were described previously herein. The chip is washed, stained, scanned for detection of phycoerythrin (PE) label, and normalized to enable comparison of data between chips to eliminate chip-to-chip variability, following standard protocol, well known to those in the art. See generally, Lockhart et al. 1996; Affymetrix GeneChip (R) Expression Analysis Technical Manual, Rev. 3 (2002). In this procedure, replicate signal intensity mean, standard deviation, and standard error measurements are computed for each of the various cell sub-populations by standard software analysis. These gene expression maps may be used to identify individual unknown cells that correspondingly express genes characteristic of pinpointed cells on the HSC -> MPP -> CMP, CLP hematopoietic cell differentiation pathway gene expression map.

As detailed in Example 12, the procedure for grouping expressed genes into clusters for each of the HSC, MPP, CMP, and CLP populations involved utilization of (1) Affymetrix analysis software, (2) Prescreening using a screening filter, and (3) K-means clustering.

FIG. 4 shows hybridization results with the majority of genes with expression levels of more than 200. A standardized, normalized gene expression level was computed to be equal to an expression level of a gene minus the mean of the expression level of the CD45+ HSC gene. All of the gene expression level changes, depicted as standard deviation color codings in FIG. 4, were translated into normalized values such that each of the means for the corresponding genes were given an assigned value of zero (“0”), with a measured standard deviation about that mean.

Normalized gene expression level changes were categorized into the following standard deviation groups: −1.5 to −0.75; −0.75 to −0.50; −0.50 to −0.25; −0.25 to 0.0; 0.0 to 0.25; 0.25 to 0.50; 0.50 to 0.75; 0.75 to 1.5. Importantly, a negative standard deviation (e.g., −1.5) indicates a very low level of expression variability relative to the expression of this particular gene under other conditions, rather than “no expression” of that gene. Note that these levels represent eight ranks. Thus, the gene expression data in FIGS. 4 and 5 may also be placed into relative ranks of −4, −3, −2, −1, 1, 2, 3, and 4.

The gene expression map with its associated gene expression level standard deviation changes for the unknown cell may be compared to the standardized gene expression maps of the HSC, MPP, CMP, and CLP sub-populations. The unknown cell can then be associated with one of these four sub-populations. Moreover, the unknown cell can be placed in a transition cell category where the unknown cell manifests gene expression characteristic of an HSC-MPP, MPP-CMP, or MPP-CLP transition cell. A typical cells (e.g., leukemia cells, tumor cells, non-hematopoietic cells) that express genes of the HSC, MPP, CMP, and/or CLP sub-populations may also be characterized by comparison with the four sub-population gene expression patterns. As more data is accumulated regarding hematopoietic transition cells, these known cells can be used as provided standard sub-populations permitting characterizations of their respective hematopoietic cell differentiation pathways.

### Example 22

A gene matrix can be constructed by adhering and affixing isolated genes and expressed sequence tag (EST) cluster nucleic acid sequences from the cell sub-populations in a particular tissue lineage pathway onto a solid phase matrix or support. Suitable multilineage cell tissue can include but are not limited to hematopoietic, nerve, muscle, kidney, and liver. The Affymetrix.RTM.417.TM. Arrayer and 427.TM. Arrayer can be used to deposit densely packed nucleic acid arrays on glass slide matrixes. See U.S. Pat. Nos. 6,040,193 and 6,136,269, incorporated by reference in their entireties herein. Suitable solid phase matrices that can be used are silica or silica-based materials, inorganic glass, functionalized glass, polymers, plastics, resins, polysaccharides, carbon, metals, polymerized Langmuir Blodgett film, Si, Ge, GaAs, GaP, SiO.sub.2, SiN.sub.4, polytetrafluoroethylene, polyvinylidenedifluoride, polystyrene, polycarbonate, or combinations thereof.

Glass slides can be pretreated with an alkaline bath consisting of 1 liter of 95% ethanol with 120 ml of water and 120 grams of sodium hydroxide for 12 hrs. Slides are washed under running water and allowed to air dry, then rinsed again with 95% ethanol. Slides can be aminated with 0.1% amino propyl-triethoxysilane for the purpose of attaching amino groups to the glass surface. Slides are exposed to the amination solution for about 5 minutes at ambient temperature on a rotary shaker. Subsequently, slides are removed and rinsed three times with 100% ethanol.

Slides are placed in 110°-120° C. vacuum oven for 20 min., then cured at room temperature for 12 hrs in an argon environment. Slides are dipped in a dimethylformamide (DMF) solution, followed by thorough washing with methylene chloride. The aminated slide surface is exposed to a 30 mM solution of NVOC-GABA (gamma amino butyric acid) NHS (N-hydroxysuccinamide) ester in DMF to attach a NVOC-GABA protective moiety to each of the amino groups. The surface is washed with a DMF, methylene chloride, and ethanol mixture. Unreacted aminopropyl silane on the glass surface are capped with acetyl groups to prevent further reaction by exposure to a 1:3 mixture of acetic anhydride in pyridine for 1 hr. Slides are washed again in DMF, methylene chloride and ethanol.

Light from an Hg—Xe arc lamp can be imaged onto the glass surface through a laser-ablated chrome-on-glass mask in direct contact with the surface. The glass surface is exposed to 5 min. illumination with 12 mW of 350 nm broadband light to activate amino groups by photolysis. Then, nucleic acid sequences are placed in contact with the slide, washed as previously, and dried for use in assays. In this manner, separate gene arrays for each cell sub-population in a multilineage differentiation pathway can be created for subsequent characterization of sub-populations. Alternatively, the gene arrays for all sub-populations of cells associated with a multilineage pathway can be placed on the same slide matrix. Affymetrix GeneChip MU-U74 (version 2—arrays A and B (Affymetrix, Santa Clara, Calif.) also can be used.

### Example 23

The method of Example 21 can be utilized for the construction of a nucleic acid library solid phase matrix or support. Specifically, a separate glass solid phase matrix can be constructed containing a library of nucleic acid sequences associated with HSC, MPP, CMP, or CLP genes. A glass matrix can be prepared that contains solely HSC nucleic acid sequences selected from a group consisting of SEQ. ID. NOs. 3428-4863. A second glass matrix can be made that contains only MPP sequences selected from a group consisting of SEQ. ID. NOs. 2076-3427. A third glass matrix can be prepared that contains only CMP sequences selected from a group consisting of SEQ. ID. NOs. 822-2075. A fourth glass matrix can be made that contains only CLP sequences selected from a group consisting of SEQ. ID. NOs. 1-821.

Solid phase matrices can also be constructed that include nucleic acid sequences associated with a combination of hematopoietic cell sub-populations. A fifth glass matrix can be prepared that contains HSC and MPP sequences selected from a group consisting of SEQ. ID. NO. 2076-4863. A sixth glass matrix can be prepared that contains MPP and CMP sequences selected from a group consisting of SEQ. ID. NOs. 822-3427. A seventh glass slide can be made that contains MPP and CLP sequences selected from a group consisting of SEQ. ID. NOs. 1-821 and 2076-3427. An eighth glass slide can be prepared that contains HSC, MPP, CMP, and CLP sequences selected from a group consisting of SEQ. ID. NO. 1-4863.

### Example 24

Computer data analysis, depicted in FIGS. 2-5, has been performed which involves the following steps: collecting a known or unknown cell's gene expression data; computing Pearson's correlation coefficient to obtain the similarity/difference distances between the cell's genes; determining the number of expressed genes; normalizing data to permit comparisons of gene expression levels between genes; performing K-means clustering on normalized data to define gene clusters; creating gene expression maps; and visualizing the maps using graphical or color bar representations.

The gene expression data collected are obtained from processed instrument signal readings of collected photons emanating from excited fluorescence of phycoerythrin-labels (PE-labels). These PE-labels are operably bound to each of the cell's cRNA probes that correspondingly hybridize and attach to each of the nucleic acids on the solid phase array matrix as described in Examples 6-10. Computer software converts the raw hybridization intensity signal readings to expression levels for each particular gene based on the comparison between the hybridization signals of perfect match and mismatch pairs among a number of nucleic acid sequences (e.g., 15-20) which characterize that given gene.

Negative values are converted to positive values by the software to facilitate further data processing. Thus, all negative values were converted to a positive 20, using 20 as the background level. Other background level numerical values, different from “20,” may be also be chosen. Pearson's correlation coefficient (PCC) determination was performed for genes of HSC, MPP, CMP, and CLP cell sub-populations using the converted numerical data to obtain gene similiarity/difference distance determinations such as those obtained in FIG. 1A. The greater the computed distance between two genes, the greater the difference between those two genes. Thus, FIG. 1A shows that the distance between HSC and MPP is 0.951. The distance between MPP and CLP is 0.935. The distance between MPP and CMP is 0.930. The distance between HSC and CLP is 0.900. The distance between HSC and CMP is 0.866.

Prior to K-means clustering analysis, the computer software defines an “expressed gene.” An “expressed” gene is defined by the software as an expression level of a given gene being more than 100. However, other expression levels may be chosen. The expression data for each “expressed gene” is then subjected to a software screening filter that selects certain expressed genes based upon pre-established filtering criteria. The filtering criteria used required (1) an absolute difference of gene expression level of greater than 100, and (2) and a fold-change of expression level for each gene of greater than 2-fold. Other filtering criteria may be used. 4863 expressed genes were obtained using these filtering criteria.

After the filtering criteria are applied, the computer software normalizes the gene expression data. A normalized or standardized gene expression level is equal to the following ratio: (an expression level of a gene minus the mean of the expression levels of this gene) divided by the standard deviation (s.d.) of the expression level of this gene. Expression levels for all the genes are normalized such that each mean is brought to a zero (0) value to permit comparisons of the standard deviations, as a measure of gene expression level variability or change. Thus, genes expressed in different sub-populations of cells can be compared by utilizing normalized standard deviation data that quantitate expression level variability.

Computer software-generated K-means clustering analysis is then performed on normalized standard deviation gene expression change data. The K-means clustering method groups genes together according to their similarity as previously discussed in Example x. Gene clusters created by this method include, but are not limited to, the 100 gene clusters of FIGS. 4 & 5, and Tables 1 & 3, and the clusters 1-8 of Table 4 and FIG. 8. Standard deviation ranges for each gene were categorized by the software into following ranges: −1.5 to −0.75, −0.75 to −0.50, −0.50 to −0.25, −0.25 to 0.0, 0.0 to 0.25, 0.25 to 0.50, 0.50 to 0.75, 0.75 to 1.5. Ranks of the standard deviation ranges can also be expressed as follows: rank 1=−1.5 to −0.75, rank 2=−0.75 to −0.50, rank 3=−0.50 to −0.25, rank 4=−0.25 to 0.0, rank 5=0.0 to 0.25, rank 6=0.25 to 0.50, rank 7=0.50 to 0.75, and rank 8=0.75 to 1.5.

The categorized gene clusters and their corresponding normalized gene expression data, expressed as standard deviations or ranks, are then utilized to create computer-generated gene expression maps corresponding to the gene clusters expressed in various cell sub-populations. Spotfire software was used to display graphical representations and color bar representations of the gene expression level change data corresponding to each gene within a gene cluster. Other software can developed or obtained to display representations of the gene expression level change data. Thus, computer-generated gene expression maps were obtained for genes expressed in HSC, MPP, CMP, and CLP sub-populations.

Representative computer software-generated gene expression maps are depicted in FIGS. 2, 3 and 4 in color bar form, and FIG. 5 in graphical form. FIG. 5 shows a graphical representation of the gene expression data. The vertical axis represents normalized gene expression standard deviation values, and the horizontal axis represents discrete HSC, MPP, CLP, and CMP sub-populations. FIG. 5 contains four separate graphs, labeled as FIG. A, B, C, and D. Each graph corresponds to a different gene cluster that contains genes possessing a similar expression pattern. FIG. 4 shows a computer software-generated color bar representation of the same gene expression data as that depicted in FIG. 5. FIG. 4A, 4B, 4C, and 4D show gene fingerprint color pattern maps that characterize gene expression for the HSC, MPP, CLP, and CMP sub-populations.

### Example 25

FIG. 8 and Table 4 show results of data from representative clusters 1 through 8, wherein the genes in each respective cluster are divided into groups that possess characteristic up-regulated and down-regulated gene expression patterns. For example, Cluster 1 includes a group of genes that are down-regulated in MPP as compared to HSC. The Cluster 1 grouping of down-regulated genes had MPP vs HSC fold changes ranging between −5 and −2. Cluster 1 includes the following genes: AML-1, CCAAT, Gut enriched Kruppel-like factor, Jun-B, Transcription factor LRG-21, Zinc finger protein 36, FMS-like tyrosine kinase 3, Phosphalidylinositol 3-kinase regulatory subunit, Ly-6E. 1 alloantigen, N10 a nuclear hormonal binding receptor, Flamingo 1, Notch-1, Cathepsin S, Retinol binding protein 1, cellular, Fibroblast growth factor 4, Small inducible cytokine A3, TGF beta-induced protein, 68 kDa, and PAC-1. When the normalized standard deviation value obtained for the AML-1 gene was compared in the MPP cell subpopulation vs. the HSC cell sub-population, the computed ratio was −2.4, indicating greater expression in the HSC sub-population in comparison to the MPP sub-population.

The Cluster 2 grouping of up-regulated genes had MPP vs. HSC fold changes ranging between 2.0 and 6.7. Cluster 2 includes the following genes: HNF-6, CDC28, JAK2, nek2, sak-b, Serine/threonine kinase 6, CD48 antigen, Male enhanced antigen 1, Acetyltransferase Tubedown-1, BMP-4, Wnt10a, Caspase-3, Cathepsin G, Hsp70, T-IAP, BUB1, Cell division cycle control protein 2a, Cyclin A2, Cyclin B1, Cyclin B2, Cyclin F, Kinesin-related mitotic motor protein, Mitotic centromere-associated kinesin, Mitotic checkpoint component Mad2, BUB1B, Rab6kill, RFC4, TERF2, Chemokine (C—C) receptor 1-like, Crlf1, FGF-2, Small inducible cytokine A9, DDX3, elF4B, Snrp116, Karyopherin (importin) alpha 2, and HMG4. The The Cluster 3 grouping of down-regulated genes had CLP vs MPP fold changes ranging between −16.8 and −2.2, and CMP vs MPP fold changes ranging between −17.9 and −2. Cluster 3 includes the following genes: ATF-4, COPEB, egr1, Jun-D, Transcription factor LRG-21, Zinc finger protein 36, RGS2, Male enhanced antigen 1, BMP-4, RhoB, Slpi, FGF-2, EEF-Tu, mEAR-1, and Ear3.

The Cluster 4 grouping of up-regulated genes had CLP vs MPP fold changes ranging between 2 and 14.2, and CMP vs MPP fold changes ranging between 2 and 7. Cluster 4 includes the following genes: CTCF, Bromodomain adjacent to zinc finger domain, 2A, CDC45-related protein, Cbfa1/Osf2/Runx2, Foxm1, Ikaros DNA binding protein, LIM-only domain transcription factor LMO-4, Mad4, Nmi, RelA, pOU domain, class 2, transcription factor 1, Taube nuss, Zfp265, JAK3, Chk2, RAN GTPase activating protein 1, Krev-1, MNBH, Smad4, STAT6, CD164, CD1d1 antigen, CD44 antigen, Fibronectin receptor beta-chain, Inositol trisphosphate receptor type 2, Ly86, Sra1, Gcdp, Notch-1, Napor-3, BH3 interacting domain death agonist, Caspase-3, Caspase-6, Caspase-8, Catalase, Hypoxia inducible factor 1, alpha subunit, Cyclin-dependent kinase homologue, mutant p53, Myb proto-oncogene, Myelocytomatosis oncogene, RAB9, member RAS oncogene family, Retinoblastoma-like 1, Lcr-1 (CXCR-4 homologue), Stromal cell derived factor receptor 1, Interferon (alpha and beta) receptor, and Phosphatase and tensin homolog.

The Cluster 5 grouping of down-regulated genes had CLP vs CMP fold changes ranging between −76.6 and −3.4, and CLP vs MPP fold changes ranging between −25.0 and −2. Cluster 5 includes the following genes: FOG, GATA-binding protein 1, GATA-binding protein 2, LIM-only domain transcription factor LMO-2, Apolipoprotein E, G-protein coupled thrombin receptor, Trfr2, Tyrosine kinase receptor 1, Cathepsin G, Myeloperoxidase, Proteinase3, Spi2 proteinase inhibitor, Retinol binding protein 1, cellular, Small inducible cytokine A9, and Neutrophil elastase.

The Cluster 6 grouping of up-regulated genes had CLP vs CMP fold changes ranging between 2 and 125.3, and CLP vs MPP fold changes ranging between 2.1 and 44.7. Cluster 6 includes the following genes: bHLH, B-cell-specific coactivator BOB.1/OBF.1, AML-1 (Cbfa1/Osf2/Runx2), Ikaros DNA binding protein, Inhibitor of DNA binding 2, Tcf7, Tusp, Cytochrome c oxidase subunit VlaH, B lymphoid kinase, FMS-like tyrosine kinase 3, Intracellular calcium-binding protein (MRP14), Intracellular calcium-binding protein (MRP8), Limk1, Rearr. lymphocyte protein-tyrosine kinase, Smad7, B cell linker protein BLNK, Interleukin 7 receptor, Interleukin-18 receptor accessory protein-like, Lymphocyte antigen 6 complex, locus D, NK cell receptor 2B4, Putative transmembrane receptor IL-IRrp, Hairy and Enhancer of Split 6, Notch-1, Cathepsin S, Pre-B lymphocyte 1, Rag-1, Rag-2, T-cell receptor beta-chain constant region, Tdt, Interleukin 12a, Lymphotoxin-beta, and Small inducible cytokine A5.

The Cluster 7 grouping of down-regulated genes had CMP vs CLP fold changes ranging between −31.7 and −2.1, and CMP vs. MPP fold changes ranging between −4.8 and −2.2. Cluster 7 includes the following genes: Gut enriched Kruppel-like factor, TGF-beta-stimulated clone-22, Cbp, Intracellular calcium-binding protein (MRP14), GABA-A, N10 a nuclear hormonal binding receptor, and Frizzled homolog B (Drosophila). The Cluster 8 grouping of up-regulated genes had CMP vs CLP fold changes ranging between 2 and 76.6, and CMP vs MPP fold changes ranging between 2 and 6.9.

Functional category definitions for each of the genes are indicated as follows: A=transcription, B=metabolism, C=signal transduction, D=antigens and receptors, E=development, F=cytoskeleton, G=apoptosis, stress, inflammation, H=cell cycle, proliferation, I=immune response, J=cytokines and growth factors, K=protein modification, interaction, L=channels and transporters, M=extracellular matrix and adhesion, N=RNA processing, O=intracellular trafficking, P=chromatin modification, DNA repair, Z=unclassified. In addition, positions of up-regulated and down-regulated genes clusters 1-8 are depicted in FIG. 8.

### Example 26

Single-cell RT-PCR amplification was executed using a procedure modified from that of Hu M, Krause D, Greaves, et al. Genes Dev. 1997; 11:774-785. Single HSC, MPP, CMP, and CLP cells were isolated to obtain amplified targeted gene sequences for analysis. In this modified procedure, (1) Single murine bone marrow-derived HSC, MPP, CMP, and CLP cells were directly triple FACS sorted, micromanipulated or otherwise deposited individually into 96 well arrays of 0.2 ml microamp tubes, and (2) the lysis buffer preferably contained 0.5% Triton X-100 buffer instead of 0.4% NP-40 buffer. After deposition of single cells into the 96 well arrays of microamp tubes and cell lysis, HSC, MPP, CMP, and CLP-derived nucleic acid molecules from each single cell are separately amplified in individual wells by the following PCR method.

Individual cells from murine bone marrow are sorted by FACS or micromanipulated into 96-well plate arrays of 0.2 ml microamp tubes containing cell lysis buffer. The accuracy of the flow sorting or micromanipulation was verified by microscopic examination of the single cells deposited into each of the 96 wells on the plate. A reverse transcriptase protocol was then performed using gene-specific primers for all the genes, ESTs and gene regions of interest. The genes, ESTs, and gene fragments used may be any identified genes of HSC, MPP, CMP, and CLP. These genes include, but are not limited to, those genes depicted in FIG. 2, FIG. 3, and FIG. 4, SEQ ID NOs. 1-4863, cumulative clusters 1-100, and representative clusters 1-8.

First round PCR was performed using 3′ gene-specific forward and reverse primers, wherein these primers include a spanned length of at least one intron. Primers were made for HPRT, MHCII.2A, ESK, and CyclinA2 tested genes. The housekeeping HPRT transcripts were used as a control to monitor the success and efficiency of the RT-PCR reaction. Aliquots of the first round PCR reaction are subsequently replicated into replicate plates. Second round PCR reactions were then performed separately for each gene with fully nested primer pairs. Upon completion of the second round PCR reactions, aliquots were subjected to agarose gel electrophoresis and visualized by ethidium bromide staining. Single cell RT-PCR results obtained are shown in FIG. 6. In FIG. 6ii, a table of the ratio of tested genes to internal HPRT control is presented for each of the HPRT, MHCII.2A, ESK, and Cyclin A2 genes. Thus, 57 HSC wells and 68 MPP wells were determined to be positive for the HPRT housekeeping control gene. For the MHCII.2A gene, 40 of 57 HSC individual cells expressed the target gene, whereas only 16 of 68 MPP cells expressed that same gene. Similarly the ESK gene was preferentially expressed in 36/68 MPP cells, as compared to lack of expression (0/57) manifested by the HSC cells.

FIG. 6i. depicts microarray results for the MHCII.2A, ESK, and CyclinA2 genes among HSC, MPP, CLP, and CMP individual cells. Results are expressed as numerical scores indicating relative measured gene expression intensity levels. For an HSC cell, the MHCII.2A gene was expressed at higher levels (329) than either the ESK (41) or CyclinA2 (69) genes. In contrast, for an MPP cell, CyclinA2 (500) was expressed at a higher level than ESK (196), which was correspondingly expressed at a higher level than MHCII.2A (83). As with the MPP cell, a CMP cell exhibited a nearly identical graded expression pattern for CyclinA2 (430), ESK (204), and MHCII.2A (76) gene markers. However, a CLP cell exhibited a reduced gene expression signal pattern for CyclinA2 (273), ESK (166) and MHCII.2A (20) genes respectively.

FIG. 6iii shows a representative agarose gel that visualizes the electrophoretic patterns for HPRT, MHCII.2A, CyclinA2, and ESK genomic bands. The HPRT cDNA-derived PCR product is 249 bp, the MHCII.2A cDNA-derived PCR product is 198 bp, the CyclinA2 cDNA-derived PCR product is 200 bp, and the ESK cDNA-derived PCR product is 197 BP. Gene expression for individual cells was characterized by deposition of cDNA-derived PCR products into separate gel lanes. HPRT gene expression was found in HSC lanes 1-7, 10, and 12; and in MPP lanes 1, 2, 4, and 6-10. MHCII.2A gene expression was detected in HSC lanes 3-10 and MPP lanes 9-11. CyclinA2 expression was observed in HSC lanes 2, 3, 5, 6, and 8-11. ESK expression was obtained in MPP lanes 1, 2, 4-11.

### Example 27

A hematopoietic cell differentiation test kit can be constructed that contains the following components: a container, a hematopoietic cell microarray or an Affymetrix GeneChip MU-U74 Array A and B; a 96-well microtiter plate array of 0.2 ml microamp tubes; fluorescence-labeled (e.g., fluorescein, rhodamine) monoclonal antibodies to HSC, MPP, CMP, and CLP markers (e.g., Thy-1, Sca-1, Lin); four sets of Dynabead packed affinity columns for purification of HSC, MPP, CMP, and CLP cells, respectively; biotinylated RNA probe positive control standards which specifically bind to HSC, MPP, CMP, and CLP regions on the arrays; preserved HSC, MPP, CMP, and CLP control cell lysates; gene specific primers for at least four genes corresponding to each four sub-populations of cells for RT-RNA replication, R-phycoerythrin-conjugated streptavidin; antisense biotinylated control cRNA (bioB, bioC, bioD, and cre); a HPRT RNA transcript positive control for RT-PCR reactions, biotin-N-hydroxysuccinimide ester; biotinylated-cRNA controls for HSC, MPP, CMP, and CLP sub-populations, Qiagen RNAeasy columns, lysis buffer containing 0.5% Triton X-100, ethidium bromide stain for electrophoresis, reference gene expression maps for HSC, MPP, CMP, CLP sub-populations and transition cells; and computer software for data analysis.

The kit's included computer software can perform the following functions: conversion of raw hybridization intensity data into gene expression levels based on computation between hybridization signals of perfect match and mismatch pairs; conversion of negative values to positive values; computation of “expressed” gene numbers, based on establishment of a minimum expression level; Pearson correlation coefficient computation for gene distance similarities and differences; prescreening and selection of genes using a screening filter for subsequent clustering analysis; normalization of gene expression level standard deviation results; K-means gene clustering for grouping of cumulative clusters 1-100 of Table 3 and representative clusters 1-8 of Table 4; conversion of normalized gene expression standard deviation results to graphical representation, and color fingerprinting among cell sub-population categories utilizing Spotfire visualization of gene expression map fingerprint patterns.

The kit user will provide a FACS sorter or micro-manipulator for separation and purification of hematopoietic cells and deposition into tubes of 96 well plate arrays; hematopoietic cells to be characterized; fluorescence detection instrumentation; Dulbecco's MEM or RPMI-1640 culture media; HEPES, cell buffers; micro-pipettors and tips; electrophoresis apparatus; and agarose gels.

### Example 28

The foregoing test kit can be used in characterization of both unknown hematopoietic genes and unknown cells along the HSC to MPP to CMP/CLP differentiation pathways. Briefly, bone marrow, spleen, liver, lymph node, or other hematopoietic stem cell sources are separated by FACS sorting or Dynabead affinity column separation into HSC, MPP, CMP, and CLP sub-populations of interest. Upon separation, isolated cells from a specific sub-population (e.g., HSC-MPP transition cells) are placed individually in a 96 well microtiter plate array using either FACS instrument sorting or micromanipulation. The HSC-MPP transition cells are lysed in cell lysis buffer, and subjected to first and second round PCR amplification to obtain amplified cRNA copies of HSC and MPP genes of interest utilizing gene-specific forward and reverse primers. Lysates containing PCR amplified c-RNA copies of HSC, MPP, CMP, and CLP genes respectively can be included in parallel with the HSC-MPP transition cell lysates as positive controls.

The amplified cRNA from replicate wells derived from the HSC-MPP transition cell and the four sub-population control cells can be biotinylated with the biotin-NHS ester and mixed with antisense biotinylated control cRNA. Then the biotinylated-specific cRNA and/or the antisense control cRNA for each respective sub-population is hybridized with the nucleic acid sequences on the microarray or the Affymetrix GeneChip. Strepavidin-conjugated phycoerythrin is added to enable detection of the gene expression level for each corresponding gene on the HSC and MPP gene expression map. The reference gene expression map is created by use of the kit's software, as provided in Example 27. Characterization of the expression pattern of a plurality of genes (e.g., at least 5 genes) from the HSC-MPP transition cell will yield a gene expression fingerprint map that may include characteristics of the HSC positive control cell and the MPP positive control reference cell genes. The gene expression fingerprint map characterizing the HSC-MPP transition cell can be compared with the reference gene expression fingerprint maps obtained from both (1) the reference HSC and MPP sub-population cell lysates included in the aforementioned biotinylation procedure, and (2) the kit's biotinylated cRNA control reagents provided for corresponding reference HSC and MPP cell sub-populations. In addition, the HSC-MPP transition cell gene expression map may be compared against the kit's included printed version of the reference HSC, MPP, CMP, CLP, and transition cell gene expression maps.

In this manner, both the isolated individual HSC-MPP transition genes and transition cells that express these genes can be characterized and identified. Similarly, isolated individual MPP to CMP transition genes and cells, as well as MPP to CLP transition genes and cells, can be characterized and identified. Moreover, individual cells within the HSC, MPP, CMP, and CLP sub-population categories may be further characterized.

For characterization of a particular unknown HSC-MPP transition gene, amplified cRNA from the isolated HSC-MPP cell can be obtained and biotinylated. This is mixed with antisense biotinylated control cRNA and hybridized with nucleic acid sequences on the microarray. Strepavidin-conjugated phycoerythrin is added, and a light source excites fluorescence photon emission from the phycoerythrin label. The instrument's detector collects the emitted light photon signal and the instrument's software converts the raw photon data into gene expression intensity signal data. The instrument's software can further convert the unknown gene expression intensity signal data to normalized gene expression data expressed as a numerical standard deviation (s.d.) value. The software then compares the unknown gene expression numerical s.d. value for the same gene's expression numerical s.d. value in the control reference HSC, MPP, CMP, CLP, and transition cell sub-populations. Based upon the unknown gene's expression numerical s.d. value software then determines similarities or differences in comparison with the control reference cell values to characterize and identify the unknown gene. Moreover, when this gene characterization procedure is executed for a plurality of genes obtained from several HSC-MPP transition cells, the kit's software can be utilized to obtain groupings of gene clusters that characterize and identify a particular HSC-MPP transition cell sub-population.

For characterization of an isolated unknown transition cell, the above procedure can be followed for each individual gene. In this manner, an unknown transition cell's gene expression map may be constructed for a plurality of individual genes. As described previously, this unknown cell's gene expression map can be compared with the reference gene expression maps for known reference HSC, MPP, CMP, CLP, and transition cell sub-populations. Based upon these map comparisons, the unknown transition cell can then be characterized and identified.

### Example 29

Standardized or normalized gene expression level values were calculated from processed gene expression data. Expression level values for all genes were processed using fluorescence signal measurement mean and standard deviation values.

A normalized or standardized gene expression level value individually was computed according to the following formula:

Gene Expression Level Value=[(an expression level of a gene)−(the mean of the expression levels of this gene)]+(s.d. of the expression level of this gene)

In the above equation, the gene expression level value is computed to equal difference between the expression level of a gene and the mean expression level of the gene divided by the standard deviation of the expression level of the gene.

Expression level values for each of the genes characterized were normalized such that each gene's mean expression level value was brought to a zero (0) value. This is illustrated as follows, assume that a gene A initially possesses a mean expression level value of 100 units, with a standard deviation of 50 units (i.e., 100±50), and gene B initially has a mean value of 200 units, with a standard deviation of 100 (i.e., 200±100). After normalization, genes A and gene B each possess a mean expression level value of zero (0). However, gene A still has a standard deviation value of 50, and gene B has a standard deviation value of 100 (i.e., A=0±50, B=0±100).

This data normalization permits comparisons of the standard deviations between genes, as a measure of gene expression level variability or change. Thus, gene A's standard deviation of 50 is a lesser change than gene B's standard deviation of 100. Standard deviations for all the genes in a gene cluster or other gene grouping may be used to generate standard error measurements that may subsequently be used to compare the gene expression level changes obtained. Thus, genes expressed in different sub-populations of cells can be compared by utilizing normalized standard deviation data that quantitate expression level variability.

Thus, there has been shown and described a novel method for determining gene expression which fulfills all the objects and advantages sought therefor. It is apparent to those skilled in the art, however, that many changes, variations, modifications, and other uses and applications for the subject method are possible, and also such changes, variations, modifications, and other uses and applications which do not depart from the spirit and scope of the invention are deemed to be covered by the invention, which is limited only by the claims which follow.

### REFERENCE LIST

1. Akashi, K., et al., Lymphoid development from stem cells and the common lymphocyte progenitors. Cold Spring Harb Symp Quant Biol, 1999. 64: p. 1-12.

Akashi, K., Traver, D., Miyamoto, T. and Weissman, I. L., A clonogenic common myeloid progenitor that gives rise to all myeloid lineages. Nature, 2000. 404 (6774): p. 193-7.

Avalos, B. R., The granulocyte colony-stimulating factor receptor and its role in disorders of granulopoiesis. Leuk Lymphoma, 1998. 28(3-4): p. 265-73.

Baylin, S., Tying it all together: epigenetics, genetics, cell cycle, and cancer. Science, 1997. 277(5334): p. 1948-9.

Biggs, W. H., 3^{rd} and W. K. Cavenee, Identification and characterization of members of the FKHR (FOX O) subclass of winged-helix transcription factors in the mouse. Mamm Genome, 2001. 12(6): p. 416-25.

Blum, S., R. E. Forsdyke, and D. R. Forsdyke, Three human homologs of a murine gene encoding an inhibitor of stem cell proliferation. DNA Cell Biol, 1990. 9(8): p. 589-602.

Brady, J. P. and J. Piatigorsky, A mouse cDNA encoding a protein with zinc-fingers and a KRAB domain shows similarity to human profilaggrin. Gene, 1994. 149(2): p. 299-304.

Busslinger, M., S. L. Nutt, and A. G. Rolink, Lineage commitment in lymphopoiesis. Curr Opin Immunol, 2000. 12(2): p. 151-8.

Chaudhary, P. M., Roninson I B, Expression and activity of P-glycoprotein, a multidrug efflux pump, in human hematopoietic stem cells. Cell, 1991. 66: p. 85-94.

Chen, X., et al., Kruppel-like factor 4 (gut-enriched Kruppel-like factor) inhibits cell proliferation by blocking G1/S progression of the cell cycle. J Biol Chem, 2001. 276(32): p. 30423-8.

Cheshier, S. H., Morrison, S. J., Liao, X. and Weissman, I. L., In vivo proliferation and cell cycle kinetics of long-term self-renewing hematopoietic stem cells. Proc Natl Acad Sci USA, 1999. 96(6): p. 3120-5.

Crosier, K. E., et al., Expression and functional analysis of two isoforms of the human GM-CSF receptor alpha chain in myeloid development and leukemia. Br J Haematol, 1997. 98(3): p. 540-8.

Dexter, T. M., M. A. Moore, and A. P. Sheridan, Maintenance of hemopoetic stem cells and production of differentiated progeny in allogeneic and semiallogeneic bone marrow chimeras in vitro. J Exp Med, 1977. 145(6): p. 1612-6.

Douville, E. M., et al., Multiple cDNAs encoding the esk kinase predict transmembrane and intracellular enzyme isoforms. Mol Cell Biol, 1992. 12(6): p. 2681-9.

Eaves, C., et al., Changes in the cytokine regulation of stem cell self-renewal during ontogeny. Stem Cells, 1998. 16 Suppl 1: p. 177-84.

Ema, H. and H. Nakauchi, Expansion of hematopoietic stem cells in the developing liver of a mouse embryo. Blood, 2000. 95(7): p. 2284-8.

Fode, C., et al., Sak, a murine protein-serine/threonine kinase that is related to the Drosophila polo kinase and involved in cell proliferation. Proc Natl Acad Sci USA, 1994. 91(14): p. 6388-92.

Ford, A. M., et al., Immunoglobulin heavy-chain and CD3 delta-chain gene enhancers are Dnase I-hypersensitive in hemopoietic progenitor cells. Proc Natl Acad Sci USA, 1992. 89(8): p. 3424-8.

Fortunel, N. O., A. Hatzfeld, and J. A. Hatzfeld, Transforming growth factor-beta: pleiotropic role in the regulation of hematopoiesis. Blood, 2000. 96(6): p. 2022-36.

Gong, S. G. and A. Kiba, The role of Xmsx-2 in the anterior-posterior patterning of the mesoderm in Xenopus laevis. Differentiation, 1999. 65(3): p. 131-40.

Hobert, O., et al., Isolation and developmental expression analysis of Enx-1, a novel mouse Polycomb group gene. Mech Dev, 1996. 55(2): p. 171-84.

Hu, M., et al., Multilineage gene expression precedes commitment in the hemopoietic system. Genes Dev, 1997. 11(6): p. 774-85.

Hume, D. A., et al., Regulation of CSF-1 receptor expression. Mol Reprod Dev, 1997. 46(1): p. 46-52; discussion 52-3.

Jacobs, J. J., et al., The oncogene and Polycomb-group gene bmi-1 regulates cell proliferation and senescence through the ink4a locus. Nature, 1999. 397(6715): p. 164-8.

Jiminez, G., et al., Activation of the beta-globin locus control region precedes commitment to the erythroid lineage. Proc Natl Acad Sci USA, 1992. 89(22): p. 10618-22.

Kautz, B., et al., SHP1 protein-tyrosinephosphatase inhibits gp91PHOX and p67PHOX expression by inhibiting interaction of PU.1, IRF1, interferon concensus sequence-binding protein, and CREB-binding protein with homologous Cis elements in the CYBB and NCF2 genes. J Biol Chem, 2001. 276(41): p. 37868-78.

Kim, M., et al., Rhodamine-123 staining in hematopoietic stem cells of young mice indicates mitochondrial activation rather than dye efflux. Blood, 1998. 91(11): p. 4106-17.

Kimura, S., et al., Hematopoietic stem cell deficiencies in mice lacking c-Mpl, the receptor for thrombopoietin. Proc Natl Acad Sci USA, 1998. 95(3): p. 1195-200.

Kiyono, T., Foster, S. A., Koop, J. I., McDougall, J. K., Galloway, D. A., Klingelhutz, A. J., Both Rb/p161NK4a inactivation and telomerase activity are required to immortalize human epithelial cells. Nature, 1998. 396(6706): p. 84-8.

Kondo, M., I. L. Weissman, and K. Akashi, Identification of clonogenic common lymphoid progenitors in mouse bone marrow. Cell, 1997. 91(5): p. 661-72.

Krull, C. a. K., R., Building from the bottom up. Nature Cell Biology, 2001. 3: p. 138-9.

Kuo, C. T. and J. M. Leiden, Transcriptional regulation of T lymphocyte development and function. Annu Rev Immunol, 1999. 17: p. 149-87.

Lagasse E., s. J., Uchida, N., Tsukamoto, A., Weissman I L, Toward Regenerative Medicine. Immunity, 2001. 14: p. 425-436.

May, G. a. E., T., The lineage commitment and self-reneal of blood stem cells. Chapter 5 in “Hematopoiesis—A developmental approach” Edited by Zon, L. I., 2001. Oxford University Press: p. 72-74.

Meraldi, P. and E. A. Nigg, Centrosome cohesion is regulated by a balance of kinase and phosphatase activities. J Cell Sci, 2001. 114(Pt 20): p. 3749-57.

Milner, L. A. and A. Bigas, Notch as a mediator of cellfate determination in hematopoiesis: evidence and speculation. Blood, 1999. 93(8): p. 2431-48.

Morrison, S. J. and I. L. Weissman, The long-term repopulating subset of hematopoietic stem cells is deterministic and isolatable by phenotype. Immunity, 1994. 1(8): p. 661-73.

Morrison, S. J. et al., Hematopoietic stem cells: challenges to expectations. Curr Opin Immunol, 1997. 9(2): p.216-21.

Okuda, T., van Deursen, J., Hiebert S. W., Grosveld, G., Downing, J. R., AML1, the target of multiple chromosomal translocations in human leukemia, is essential for normal fetal liver hematopoiesis. Cell, 1996. 84: p. 321-30.

Park, I., He, Y., Lin, F., Lacrum, O. D., Tian, Q., Bumgarner, R., Klug, C., Li, K., Kuhr, C., Doyle, M., Xie, T., Schummer, M., Sun, Y., Goldsmith, A., Clarke, M. F., Weissman, I. L., Hood, L. and Li, L., Differential Gene Expression Profiling of Adult Murine Hematopoietic Stem Cells. Blood, 2001. In press.

Porcher, C., Swat, W., Rockwell, K., Fujiwara, Y., Alt, F. W., Orkin, S. H., The T cell leukemia oncoprotein SCL/tal-1 is essential for development of all hematopoietic lineages. Cell, 1996. 86: p. 47-57.

Ray, D., et al., Characterization of Spi-B, a transcription factor related to the putative oncoprotein Spi-1/PU.1. Mol Cell Biol, 1992. 12(10): p. 4297-304.

Ray-Gallet, D., A. Tavitian, and F. Moreau-Gachelin, An alternatively spliced isoform of the Spi-B transcription factor. Biochem Biophys Res Commun, 1996. 223(2): p. 257-63.

Robb, L., Elwood, N. J., Elefanty, A. G., Kontgen, F., Li, R., Barnett, L. D., Begley, C. G., The scl gene product is required for the generation of all hematopoietic lineages in the adult mouse. EMBO J, 1996. 15: p. 4123-9.

Robey, E., Regulation of T cellfate by notch. Annu Rev Immunol, 1999. 17: p. 283-95.

Rolink, A. G. and F. Melchers, Precursor B cells from Pax-5-deficient mice-stem cells for macrophages, granulocytes, osteoclasts, dendritic cells, natural killer cells, thymocytes and T cells. Curr Top Microbiol Immunol, 2000. 251: p. 21-6.

Roose, J., et al., Synergy between tumor suppressor APC and the beta-catenin-Tcf4 target Tcf1. Science, 1999. 285(5435): p. 1923-6.

Rothenberg, E. V., J. C. Telfer, and M. K. Anderson, Transcriptional regulation of lymphocyte lineage commitment. Bioessays, 1999. 21(9): p. 726-42.

Roussel, M. F., Signal transduction by the macrophage-colony-stimulating factor receptor (CSF-1R). J Cell Sci Suppl, 1994. 18: p. 105-8.

Shimada, Y., et al., Asymmetric colocalization of Flamingo, a seven-pass transmembrane cadherin, and Dishevelled in planar cell polarization. Curr Biol, 2001. 11(11): p. 859-63.

Shivdasani, R. A., Orkin, S. H., The transcriptional control of hematopoiesis. Blood, 1996. 87: p. 4025-39.

Socolovsky, M., H. F. Lodish, and G. Q. Daley, Control of hematopoietic differentiation: lack of specificity in signaling by cytokine receptors. Proc Natl Acad Sci USA, 1998. 95(12): p. 6573-5.

Taipale, J. and P. A. Beachy, The Hedgehog and Wnt signaling pathways in cancer. Nature, 2001. 411(6835): p. 349-54.

Taylor, A. and K. Namba, In vitro induction of CD25 CD4 regulatory T cells by the neuropeptide alpha-melanocyte stimulating hormone (alpha-MSH). Immunol Cell Biol, 2001. 79(4): p. 358-67.

Tenen, D. G., et al., Transcription factors, normal myeloid development, and leukemia. Blood, 1997. 90(2): p.489-519.

Usui, T., et al., Flamingo, a seven-pass transmembrane cadherin, regulates planar cell polarity under the control of Frizzled. Cell, 1999. 98(5): p. 585-95.

Varnum-Finney, B., Xu, L., Brashem-Stein, C., Nourigat, C., Flowers, D., Bakkour, S., Pear, W. S., Bernstein, I. D., Pluripotent, cytokine-dependent, hematopoietic stem cells are immortalized by constitutive notchI signaling. Nat Med, 2000. 6(11): p. 1278-81.

Wang, Q., Stacy, T., Binder, M., Marin-Padilla, M., Sharpe, A. H., Speck, N. A., Disruption of the Cbfa2 gene causes necrosis and hemorrhaging in the central nervous system and blocks definitive hematopoiesis. Proc Natl Acad Sci USA, 1996. 93: p. 3444-9.

Watt, S. M., and Visser, J. W. M., Recent advances in the growth and isolation of primitive human haemopoietic progenitor cells. Cell Proliferation, 1992. 25: p. 263-297.

Weissman, I. L., Development switches in the immune system. Cell, 1994. 76: p.207-218.

Wolf, N. S., Kone, A., Priestley, G. V., Bartelmez, S. H., In vivo and in vitro characterization of long-term repopulating primitive hematopoietic cells isolated by sequential Hoechst 33342-Rhodamine 123 FACS selection. Exp Hematol, 1993. 21: p. 614-22.

Yang, Y. C., Interleukin-11 (IL-11) and its receptor: biology and potential clinical applications in thrombocytopenic states. Cancer Treat Res, 1995. 80: p. 321-40.

Youn, B. S., et al., A novel chemokine, macrophage inflammatory protein-related protein-2, inhibits colony formation of bone marrow myeloid progenitors. J. Immunol, 1995. 155(5): p. 2661-7.

What is claimed is:

1. A method for classifying an unknown multilineage-affiliated gene, comprising:
(a) isolating a population of cells;
(b) separating the population of cells into discrete cell sub-populations;
(c) isolating expressed nucleic acid sequences from the discrete cell sub-populations and forming labeled nucleic acid probes from the expressed nucleic acid sequences;
(d) hybridizing the labeled nucleic acid probes with a nucleic acid sequence library on an array, wherein identity and intensity of expression of the expressed nucleic acid sequences are identified to provide gene expression hybridization data;
(e) converting the gene expression hybridization data into a graphical representation, whereby change in gene expression between the discrete cell sub-populations is profiled;
(f) isolating a multilineage-affiliated gene of unknown identity;
(g) determining the unknown gene's expression intensity in each of the discrete sub-populations; and,
(h) comparing the unknown gene's expression pattern with known gene expression patterns in the graphical representation to associate the unknown gene with a group of known genes.

2. The method of claim 1, wherein the discrete sub-populations are selected from the group consisting of HSC, MPP, CMP, and CLP.

3. The method of claim 1, wherein the discrete sub-populations comprise HSC, transitional, and differentiated cells, wherein the differentiated cells are selected from the group consisting of adult, embryonic, neonatal, fetal liver, bone marrow, hematopoictic, splenic, and lymphoid stem cells.

4. The method of claim 1, wherein the discrete cell sub-populations are separated using cell surface markers.

5. The method of claim 1, wherein the expressed nucleic acid sequences comprise non-hematopoietic and hematopoietic genes.

6. The method of claim 1, wherein the expressed nucleic acid sequences are selected from the group consisting of RNA, DNA, and EST nucleic acid sequences.

7. The method of claim 2, wherein the HSC sub-population is identified by expression of genes selected from the group consisting of SEQ ID NOs. 3428-4863.

8. The method of claim 2, wherein the MPP sub-population is identified by expression of genes selected from the group consisting of SEQ ID NOs. 2076-3427.

9. The method of claim 2, wherein the CMP sub-population is identified by expression of genes selected from the group consisting of SEQ ID NOs. 822-2075.

10. The method of claim 2, wherein the CLP sub-population is identified by expression of genes selected from the group consisting of SEQ ID NOs. 1-821.

11. The method of claim 1, wherein the array comprises a substrate and nucleic acid sequences affixed to the substrate, wherein the nucleic acid sequences are selected from the group consisting of SEQ ID NOs. 1-4863.

12. The method of claim 1, wherein the gene expression hybridization data is converted to expression level data, whereby the expression level data are determined by comparing hybridization signals for perfect matches and mismatches of the nucleic acid sequences on the array to provide expression level data for each of the nucleic acid sequences.

13. The method of claim 12, wherein the expression level data is normalized to provide normalized expression data.

14. The method of claim 13, wherein the normalized expression data is filtered using a filter equation given by |yⱼ₍ₘ₎−yⱼ₍ₗ₎|>100 and yⱼ₍ₘ₎/yⱼ₍ₗ₎>2 for j=1, . . . ,n, where yⱼ₍ₘ₎ and yⱼ₍ₗ₎ are the order statistics with yⱼ₍ₗ₎≦ . . . ≦yⱼ₍ₘ₎ for the j^{th} gene, whereby this filtering criterion considers simultaneously the absolute difference (>100) of the gene expression levels and the fold change (>2-fold) of the expression levels for each gene (>100) to produce filtered gene expression data.

15. The method of claim 12, wherein the expression level data for the populations is statistically treated using similarity distance measurements to determine similarity of expressed genes in each population.

16. The method of claim 15, wherein Pearson correlation coefficient is used to compute gene expression intensity and diversity between the discrete sub-populations to establish a statistical measure of expression between sub-populations.

17. The method of claim 14, wherein the filtered expression data is organized using hierarchial clustering.

18. The method of claim 17, wherein the hierarchial clustering is achieved using K-means clustering.

19. The method of claim 1, wherein the graphical representation is made using Eisen software.

20. The method of claim 1, wherein the multilineage-affiliated genes are selected from the group consisting of hematopoietic and non-hematopoietic nucleic acid sequences.

21. The method of claim 18, wherein the K-means clustering method derives representative clusters 1-8.

22. The method of claim 18, wherein the K-means clustering method derives cumulative clusters 1-100.

23. The method of claim 14, wherein the filtered data is selected from SEQ ID NOs 1-4863.

24. A method for characterizing an unknown multilineage-affiliated gene comprising:
(a) profiling multilineage-affiliated gene expression in discrete cell sub-populations to provide expression data for selected genes in at least two discrete cell populations;
(b) isolating an unknown multilineage-affiliated gene;
(c) determining the unknown gene's expression characteristics for each of the sub-populations; and,
(d) comparing the unknown gene's expression data with the expression data.

25. A method for classifying an unknown multilineage-affiliated gene, comprising:
(a) isolating an unknown multilineage-affiliated gene;
(b) determining the unknown gene's expression characteristics in at least two discrete cell sub-populations; and,
(c) comparing the unknown gene's expression characteristics with profiled gene expression data for the cell sub-populations.

26. A method for determining cell stage commitment by comparing nucleic acid expression patterns, comprising:
(a) isolating a population of cells;
(b) separating the population of cells into discrete cell sub-populations;
(c) isolating expressed nucleic acid sequences from the discrete cell sub-populations and forming labeled nucleic acid probes from the expressed nucleic acid sequences;
(d) hybridizing the labeled nucleic acid probes with a nucleic acid sequence library on an array, wherein identity and intensity of expression of the expressed nucleic acid sequences are identified to provide gene expression hybridization data;
(e) converting the gene expression hybridization data into a graphical representation, whereby change in gene expression between the discrete cell sub-populations is profiled;
(f) isolating a cell of unknown commitment;
(g) obtaining gene hybridization data for the unknown cell;
(h) organizing the gene hybridization data, whereby identity of expressed genes is determined along with expression level of the identified genes; and,
(i) comparing the unknown cell's hybridization data with the gene expression data to determine the cell's commitment.

27. The method of claim 26, wherein the graphical representation is a gene cluster expression map.

28. The method of claim 26, wherein the cell sub-populations are selected from the group consisting of CMP, CLP, HSC, and MPP.

29. The method of claim 26, wherein the gene expression hybridization data is normalized to provide expression data, whereby expression of each nucleic acid sequence is standardized relative to all the nucleic acid sequences.

30. The method of claim 26, wherein the normalized expression data is filtered to group genes having similar expression levels.

31. The method of claim 26, wherein the gene expression hybridization data is converted to an expression level which is a comparison of hybridization signals for perfect matches and mismatches to provide average expression levels for the genes.

32. The method of claim 26, wherein the gene expression data for the populations is statistically treated using similarity distance measurements to determine similarity of expressed genes in each population.

33. The method of claim 26, wherein Pearson correlation coefficient is used to plot gene expression intensity and diversity between the discrete sub-populations to establish a statistical measure of expression between sub-populations.

34. The method of claim 26, wherein the normalized gene expression data is organized using hierarchial clustering.

35. The method of claim 26, wherein the hierarchial clustering is achieved using K-means clustering.

36. The method of claim 26, wherein the graphical representation is made using Eisen software.

37. The method of claim 26, wherein the discrete sub-populations comprise HSC, transitional, and differentiated cells, wherein the differentiated cells are selected from the group consisting of adult, embryonic, neonatal, fetal liver, bone marrow, hematopoietic, splenic, and lymphoid stem cells.

38. A method for predicting cell stage commitment comprising:
(a) profiling multilineage-affiliated gene expression in discrete cell sub-populations to provide reference expression data for selected genes in at least two cell populations;
(b) identifying a cell of unknown commitment;
(c) determining gene expression level patterns for the unknown cell to provide gene identity and expression level data; and,
(d) comparing the unknown cell's gene expression level data with the reference expression data.

39. A method for predicting potential of an unknown gene comprising:
(a) isolating an unknown cell;
(b) determining the unknown cell's expression characteristics in at least two cell sub-populations; and,
(c) comparing the unknown cell's expression characteristics with profiled gene expression data for the cell sub-populations.

40. A method for developing a gene expression map, comprising:
(a) isolating at least two sub-populations of cells;
(b) obtaining gene hybridization data, including gene identity data and gene expression intensity data, wherein the genes are multilineage-affiliated genes; and,
(c) converting the gene hybridization expression data to a graphical illustration.

41. The method of claim 40, wherein the graphical illustration is derived using hierarchial clustering.

42. The method of claim 40, wherein the graphical representation is made using Eisen software.

43. The method of claim 40, wherein the discrete sub-populations are selected from the group consisting of HSC, MPP, CMP, and CLP.

44. The method of claim 40, wherein the discrete sub-populations comprise HSC, transitional, and differentiated cells, wherein the differentiated cells are selected from the group consisting of adult, embryonic, neonatal, fetal liver, bone marrow, hematopoietic, splenic, and lymphoid stem cells.

45. The method of claim 40, wherein the discrete cell sub-populations are separated using cell surface markers.

46. The method of claim 40, wherein the gene expression hybridization data is converted to expression level data, whereby the expression level data are determined by comparing hybridization signals for perfect matches and mismatches of the nucleic acid sequences on the array to provide average expression levels for each of the nucleic acid sequences.

47. The method of claim 46, wherein the expression level data is normalized to provide expression data.

48. The method of claim 47, wherein the normalized expression data is filtered to group genes having similar expression levels.

49. The method of claim 46, wherein the normalized expression data is filtered using a filter equation given by |yⱼ₍ₘ₎−yⱼ₍ₗ₎|>100 and yⱼ₍ₘ₎/yⱼ₍ₗ₎>2 for j=1, . . . ,n, where yⱼ₍ₘ₎ and yⱼ₍ₗ₎ are the order statistics with yⱼ₍ₗ₎≦ . . . ≦yⱼ₍ₘ₎ for the j^{th} gene, whereby this filtering criterion considers simultaneously the absolute difference (>100) of the gene expression levels and the fold change (>2-fold) of the expression levels for each gene (>100) to produce filtered expression data.

50. The method of claim 46, wherein the expression level data for the populations is statistically treated using similarity distance measurements to determine similarity of expressed genes in each population.

51. The method of claim 50, wherein Pearson correlation coefficient is used to compute gene expression intensity and diversity between the discrete sub-populations to establish a statistical measure of expression between sub-populations.

52. The method of claim 48, wherein the filtered expression data is organized using hierarchial clustering.

53. The method of claim 52, wherein the hierarchial clustering is achieved using K-means clustering.

54. The method of claim 40, wherein the multilineage-affiliated genes are selected from the group consisting of hematopoietic and non-hematopoietic nucleic acid sequences.

55. A method for forming a hierarchial gene clustering map comprising:
(a) isolating at least two sub-populations of cells;
(b) obtaining gene hybridization data, including gene identity data and gene expression intensity data, wherein the genes are multilineage-affiliated genes;
(c) normalizing the gene hybridization expression data to provide expression data;
(d) filtering the normalized expression data to group genes having similar expression levels;
(e) organizing the normalized expression data using hierarchal clustering; and,
(f) converting the hierarchal clustering data to a graphical illustration.

56. The method of claim 55, wherein the map has an axis with standard deviation values derived from the normalized gene expression values and an axis with at least two cell populations.

57. The method of claim 55, wherein the gene expression between sub-populations is represented by a standard deviation in expression between genes in one sub-population versus another, whereby up-regulation and down-regulation are represented.

58. The method of claim 55, wherein the standard deviation values range between −1.5 and +1.5, whereby −1.5 represents down-regulation and +1.5 represents up-regulation.

59. The method of claim 55, wherein filtered expression data selects nucleic acid sequences selected from the group consisting of SEQ ID NOs. 1-4863.

60. The method of claim 55, wherein the clusters are selected from the group consisting of representative clusters 1-8.

61. The method of claim 55, wherein the clusters are selected from the group consisting of cumulative clusters 1-100.

62. An array comprising a plurality of nucleic acid sequences affixed to a substrate, wherein the nucleic acid sequences are selected from the group consisting of representative clusters 1-8 and cumulative clusters 1-100.

63. An array comprising a plurality of nucleic acid sequences affixed to a substrate, wherein the nucleic acid sequences are selected from the group consisting of SEQ ID NOs. 1-4863.

64. An array comprising a plurality of nucleic acid sequences affixed to a substrate, wherein the nucleic acid sequences are selected from the group consisting of SEQ ID NOs. 3428-4863.

65. An array comprising a plurality of nucleic acid sequences affixed to a substrate, wherein the nucleic acid sequences are selected from the group consisting of SEQ ID NOs. 1-821.

66. An array comprising a plurality of nucleic acid sequences affixed to a substrate, wherein the nucleic acid sequences are selected from the group consisting of SEQ ID NOs. 2076-3427.

67. An array comprising a plurality of nucleic acid sequences affixed to a substrate, wherein the nucleic acid sequences are selected from the group consisting of SEQ ID NOs. 822-2075.

68. A kit for characterizing a gene of unknown function by associating it with genes of HSC, MPP, CMP, and CLP reference cell sub-populastions comprising:
(a) a container;
(b) at least one nucleic acid sequence array selected from the arrays of claims 62-67; and,
(c) an activated label.

69. The kit of claim 68, wherein the solid phase matrix is selected from the group consisting of glass, silicon, plastic, and semi-conductor material.

70. A group of nucleic acid sequences for use in determining cell commitment comprising SEQ ID NOs. 1-4863.

71. A group of nucleic acid sequences representative of HSC, comprising SEQ ID NOs. 3428-4863.

72. A group of nucleic acid sequences representative of CLP, comprising SEQ ID NOs. 1-821.

73. A group of nucleic acid sequences representative of MPP, comprising SEQ ID NOs. 2076-3427.

74. A group of nucleic acid sequences representative of CMP, comprising SEQ ID NOs. 822-2075.

75. A gene cluster for use in analyzing cell differentiation selected from the group consisting of cumulative gene clusters 1-100.

76. The gene cluster of claim 75, wherein nucleic acid sequences of SEQ ID NOs. 14863 form the cumulative gene clusters.

77. A gene cluster selected from the group consisting of representative gene clusters 1-8.

78. A population of non-hematopoiesis-affiliated genes, which comprise genes listed in FIG. 3.

79. A population of genes upregulated in CMP, comprising genes listed in FIG. 4D.

80. A population of genes upregulated in HSC, comprising genes listed in FIG. 4A.

81. A population of genes upregulated in CLP, comprising genes listed in FIG. 4C.

82. A population of genes upregulated in MPP, comprising genes listed in FIG. 4B.

83. A gene cluster map for use in analysis of multilineage-affiliated genes, comprising:
(a) an axis related to at least two cell populations;
(b) an axis comprising normalized gene expression values; and,
(c) a plot of genes clustered according to K-means clustering.

84. The gene cluster map of claim 83, wherein the normalized gene expression values are computed as standard deviations of a normalized value of gene expression, where the computation on expression level of a gene minus means of expression levels of this gene's standard deviation of the expression level.

85. The gene cluster map of claim 83, wherein normalized gene expression values are figured by a comparison between hybridization signals of a PM and a MM.

86. The gene cluster map of claim 83, wherein the standard deviation ranges between −1.5 and 1.5.

87. The gene cluster map of claim 83, wherein the normalized gene expression values are screened for clustering analysis by filtering.

88. A method for gene expression profiling, comprising:
(a) isolating a population of cells;
(b) separating the population of cells into discrete cell sub-populations;
(c) isolating expressed nucleic acid sequences from the discrete cell sub-populations and forming labeled nucleic acid probes from the expressed nucleic acid sequences;
(d) hybridizing the labeled nucleic acid probes with a nucleic acid sequence library on an array, wherein identity and intensity of expression of the expressed nucleic acid sequences are identified to provide gene expression hybridization data; and,
(e) converting the gene expression hybridization data into a graphical representation, whereby change in gene expression between the discrete cell sub-populations is profiled.

89. A computer system comprising:
(a) a storage device having stored therein a gene data expression routine for compiling gene expression signal data for one or more signals associated with hybridization of labeled nucleic acid probes with a nucleic acid sequence library on an array for an available set of expressed genes;
(b) a processor coupled to the storage device for executing the gene data expression routine to determine the intensity of gene expression for each of a plurality of discrete sub-populations comprising the steps of:
(i) collecting identity and expression intensity information to construct gene expression patterns;
(ii) mapping gene expression patterns to form gene cluster expression maps for each of the discrete cell sub-populations;
(iii) determining an unknown gene's expression intensity in each of the discrete sub-populations to determine the expression level of the gene in each of the discrete sub-populations;
(iv) comparing the unknown gene's expression pattern with the gene cluster expression maps; and
(v) associating the unknown gene with one of the gene clusters to classify the unknown gene.

90. A method for determining cell stage commitment, comprising:
(a) isolating a population of cells;
(b) separating the population of cells into discrete cell sub-populations;
(c) isolating expressed nucleic acid sequences from the discrete cell sub-populations and forming labeled nucleic acid probes from the expressed nucleic acid sequences;
(d) hybridizing the labeled nucleic acid probes with a nucleic acid sequence library on an array, wherein identity and intensity of expression of the expressed nucleic acid sequences are identified to provide gene expression hybridization data;
(e) converting the gene expression hybridization data into normalized expression data, whereby change in gene expression between the discrete cell sub-populations is profiled;
(f) isolating a multilineage-affiliated gene of unknown identity;
(g) determining the unknown gene's expression intensity in each of the discrete sub-populations; and,
(h) comparing the unknown gene's expression pattern with known gene expression patterns in the graphical representation to associate the unknown gene with a group of known genes.

91. A method for classifying an unknown multilineage-affiliated gene, comprising:
(a) isolating a population of cells;
(b) separating the population of cells into discrete cell sub-populations;
(c) isolating expressed nucleic acid sequences from the discrete cell sub-populations and forming labeled nucleic acid probes from the expressed nucleic acid sequences;
(d) hybridizing the labeled nucleic acid probes with a nucleic acid sequence library on an array, wherein identity and intensity of expression of the expressed nucleic acid sequences are identified to provide gene expression hybridization data;
(e) converting the gene expression hybridization data into normalized expression data, whereby change in gene expression between the discrete cell sub-populations is profiled;
(f) isolating a multilineage-affiliated gene of unknown identity;
(g) determining the unknown gene's expression intensity in each of the discrete sub-populations; and,
(h) comparing the unknown gene's expression pattern with known gene expression patterns in the normalized expression data to associate the unknown gene with a group of known genes.

92. A method for developing a gene expression map, comprising:
(a) isolating at least two sub-populations of cells;
(b) obtaining gene hybridization data, including gene identity data and gene expression intensity data, wherein the genes are multilineage-affiliated genes; and,
(c) converting the gene hybridization expression data to normalized gene expression data.
